(19) United States
(12) Patent Application Publication
(10) Pub. No.: US 2017/0066795 A1
(43) Pub. Date: Mar. 9, 2017

(54) SOLID PRODRUG FORMS OF 2'-CHLORO-2'-METHYL URIDINE FOR HCV

(71) Applicants: IDENIX PHARMACEUTICALS LLC, Cambridge, MA (US); Merck Sharp & Dohme Corp., Rahway, NJ (US)

(72) Inventors: John Zoltan Sulliva-Bolyai, Westport Point, MA (US); Benjamin Alexander Mayes, Boston, MA (US); Douglas Lytle Mayers, Malden, MA (US); Xiao-Jian Zhou, Arlington, MA (US); Adel M. Moussa, Burlington, MA (US); Alistair James Stewart, Lincoln, MA (US); Sindhura Ganga, Waltham, MA (US); Clinton Scott Shultz, Maplewood, NJ (US); Alfred Lee, Robbinsville, NJ (US)

(73) Assignees: IDENIX PHARMACEUTICALS LLC, Cambridge, MA (US); Merck Sharp & Dohme Corp., Rahway, NJ (US)

(21) Appl. No.: 15/123,124

(22) PCT Filed: Mar. 5, 2015
(86) PCT No.: PCT/US2015/019021

§ 371 Date: Sep. 1, 2016

Related U.S. Application Data
(60) Provisional application No. 61/948,531, filed on Mar. 5, 2014.
(60) Provisional application No. 61/976,476, filed on Apr. 7, 2014.
(60) Provisional application No. 61/992,780, filed on May 13, 2014.
(60) Provisional application No. 61/993,620, filed on May 15, 2014.

(51) Int. Cl. C07H 19/10; A61K 9/48; A61K 31/7072; A61K 9/20

(52) U.S. Cl. CPC C07H 19/10; A61K 9/20; A61K 9/48; A61K 31/7072; C07B 2200/13

(57) ABSTRACT

Provided herein are compounds, compositions and methods for the treatment of Flaviviridae infections, including HCV infections. In certain embodiments, compounds and compositions of nucleoside derivatives are disclosed, which can be administered either alone or in combination with other anti-viral agents. In certain embodiments, the compounds are solid forms of Compound I: (Compound I)

### FIELD

Provided herein are compounds, methods and pharmaceutical compositions for use in treatment of viral infections, including hepatitis C virus infections in hosts in need thereof.

### BACKGROUND

The hepatitis C virus (HCV) is the leading cause of chronic liver disease worldwide. (Boyer, N. et al., J. Hepatol. 32:98-112, 2000). HCV causes a slow growing viral infection and is the major cause of cirrhosis and hepatocellular carcinoma (Di Besceglie, A. M. and Bacon, B. R., Scientific American, October: 80-85, 1999; Boyer, N. et al., J. Hepatol. 32:98-112, 2000).

About 15 to 30% of patients with chronic hepatitis due to HCV develop cirrhosis within about 20 years. (Hepatitis C Fact Sheet, World Health Organization Fact Sheet No., 164, April 2014). Development of cirrhosis due to HCV also increases the risk of hepatocellular cancer (The Merck Manual Online, Chronic Hepatitis, available at www.merckmanuals.com/professional/hepatic_and_biliary_disorders/hepatitis/chronic_hepatitis.html, last revision February 2014).

In light of the fact that HCV infection has reached epidemic levels worldwide, and has tragic effects on the infected patient, there remains a strong need to provide new effective pharmaceutical agents to treat hepatitis C that have low toxicity to the host. Further, given the rising threat of other flaviviridae infections, there remains a strong need to provide new effective pharmaceutical agents that have low toxicity to the host. Therefore, there is a continuing need for effective treatments of flavivirus infections and HCV infections.

### SUMMARY

The present application includes pharmaceutical compositions containing different amounts of Compound I or a pharmaceutically acceptable salt thereof, different forms of Compound I or a pharmaceutically salt thereof, and the use of Compound I or a pharmaceutically acceptable salt thereof to treat a flavivirus infection, such as an HCV infection in a human. Compound I has the following structure:

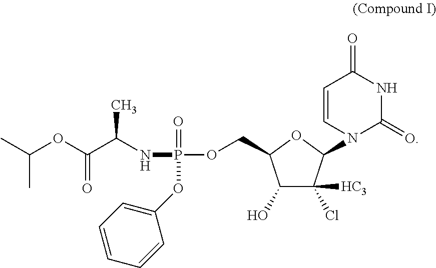

### BRIEF DESCRIPTION OF THE DRAWINGS

FIG. 1 provides an exemplary XRPD diffractogram of a sample of Form I of Compound I.

FIG. 2 depicts exemplary maximal viral load reductions in an example where the subjects were treated with Compound I in the amount of 300 mg/day for 7 days.

FIG. 3 depicts exemplary mean single-dose plasma pharmacokinetic (PK) profiles of Compound I in healthy volunteers and HCV-infected subjects in an exemplary study.

FIG. 4 depicts exemplary mean single-dose plasma PK profiles of the Nucleoside metabolite of Compound I in healthy volunteers and HCV-infected subjects in an exemplary study.

FIG. 5 depicts exemplary mean plasma PK profiles of Compound I and the Nucleoside metabolite of Compound I after multiple doses of 300 mg QD for 7 days in healthy volunteers in an exemplary study.

FIG. 6 provides an exemplary XRPD diffractogram of a sample of Form IV of Compound I.

FIG. 7 provides an exemplary XRPD diffractogram of a sample of Form V of Compound I.

FIG. 8 provides an exemplary XRPD diffractogram of a sample of Form VI of Compound I.

FIG. 9 provides an exemplary XRPD diffractogram of a sample of Form VII of Compound I.

FIG. 10 provides an exemplary XRPD diffractogram of a sample of Form VIII of Compound I.

FIG. 11 provides a solid state carbon-13 CPMAS NMR spectrum of Form I where spinning sidebands are indicted by asterisks.

FIG. 12 provides a solid state carbon-13 CPMAS NMR spectrum of Form IV where spinning sidebands are indicted by asterisks.

### DESCRIPTION OF EXEMPLARY EMBODIMENTS

### Definitions

When referring to the compounds provided herein, the following terms have the following meanings unless indicated otherwise. Unless defined otherwise, all technical and scientific terms used herein have the same meaning as is commonly understood by one of ordinary skill in the art. In the event that there is a plurality of definitions for a term herein, those in this section prevail unless stated otherwise.

As used herein, the term “samatasvir” refers to the compound identified by the chemical name “Carbamic acid, N-[(1R)-2-[(2S)-2-[5-[4-[6-[2-[(2S)-1-[(2S)-2-[(methoxycarbonyl)amino]-3-methyl-1-oxobutyl]-2-pyrrolidinyl]-1H-benzimidazol-6-yl]thieno[3,2-b]thien-3-yl]phenyl]-1H-imidazol-2-yl]-1-pyrrolidinyl]-2-oxo-1-phenylethyl]-, methyl ester” or “methyl N-{(1R)-2-[(2S)-2-{5-[4-(6-{2-[(2S)-1-{(2S)-2-[(methoxycarbonyl)amino]-3-methylbutanoyl}pyrrolidin-2-yl]-3H-benzimidazol-5-yl}thieno[3,2-b]thiophen3-yl)phenyl]-1H-imidazol-2-yl}pyrrolidin-1-yl]-2-oxo-1-phenylethyl}carbamate” and Chemical Abstracts Registry number 1312547-19-5. The chemical structure of samatasvir is provided below:

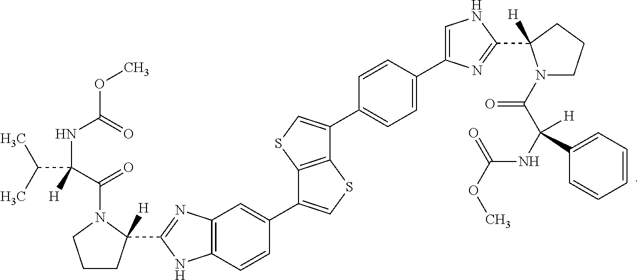

Samatasvir, including synthesis thereof, is described in U.S. Pat. No. 8,362,068 B2, the contents of which are hereby incorporated by reference in their entirety. The term “samatasvir” includes isotopic variants thereof, pharmaceutically acceptable salts thereof, and pharmaceutically acceptable solvates thereof.

“Nucleoside metabolite” refers to the following compound:

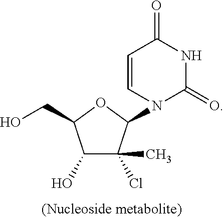 [SMILES: C[C@@]1(Cl)[C@H](O)[C@@H](CO)O[C@H]1n1ccc(=O)[nH]c1=O]

“Pharmaceutically acceptable salt” refers to any salt of a compound provided herein which retains its biological properties and which is not toxic or otherwise undesirable for pharmaceutical use. Such salts may be derived from a variety of organic and inorganic counter-ions well known in the art. Such salts include, but are not limited to: (1) acid addition salts formed with organic or inorganic acids such as hydrochloric, hydrobromic, sulfuric, nitric, phosphoric, sulfamic, acetic, trifluoroacetic, trichloroacetic, propionic, hexanoic, cyclopentylpropionic, glycolic, glutaric, pyruvic, lactic, malonic, succinic, sorbic, ascorbic, malic, maleic, fumaric, tartaric, citric, benzoic, 3-(4-hydroxybenzoyl)benzoic, picric, cinnamic, mandelic, phthalic, lauric, methanesulfonic, ethanesulfonic, 1,2-ethane-disulfonic, 2-hydroxyethanesulfonic, benzenesulfonic, 4-chlorobenzenesulfonic, 2-naphthalenesulfonic, 4-toluenesulfonic, camphoric, camphorsulfonic, 4-methylbicyclo[2.2.2]-oct-2-ene-1-carboxylic, glucoheptonic, 3-phenylpropionic, trimethylacetic, tert-butylacetic, lauryl sulfuric, gluconic, benzoic, glutamic, hydroxynaphthoic, salicylic, stearic, cyclohexylsulfamic, quinic, muconic acid and the like acids; or (2) base addition salts formed when an acidic proton present in the parent compound either (a) is replaced by a metal ion, e.g., an alkali metal ion, an alkaline earth ion or an aluminum ion, or alkali metal or alkaline earth metal hydroxides, such as sodium, potassium, calcium, magnesium, aluminum, lithium, zinc, and barium hydroxide, ammonia or (b) coordinates with an organic base, such as aliphatic, alicyclic, or aromatic organic amines, such as ammonia, methylamine, dimethylamine, diethylamine, picoline, ethanolamine, diethanolamine, triethanolamine, ethylenediamine, lysine, arginine, ornithine, choline, N,N′-dibenzylethylene-diamine, chloroprocaine, diethanolamine, procaine, N-benzylphenethylamine, N-methylglucamine piperazine, tris(hydroxymethyl)-aminomethane, tetramethylammonium hydroxide, and the like.

Pharmaceutically acceptable salts further include, by way of example only and without limitation, sodium, potassium, calcium, magnesium, ammonium, tetraalkylammonium and the like, and when the compound contains a basic functionality, salts of non-toxic organic or inorganic acids, such as hydrohalides, e.g. hydrochloride and hydrobromide, sulfate, phosphate, sulfamate, nitrate, acetate, trifluoroacetate, trichloroacetate, propionate, hexanoate, cyclopentylpropionate, glycolate, glutarate, pyruvate, lactate, malonate, succinate, sorbate, ascorbate, malate, maleate, fumarate, tartarate, citrate, benzoate, 3-(4-hydroxybenzoyl)benzoate, picrate, cinnamate, mandelate, phthalate, laurate, methanesulfonate (mesylate), ethanesulfonate, 1,2-ethane-disulfonate, 2-hydroxyethanesulfonate, benzenesulfonate (besylate), 4-chlorobenzenesulfonate, 2-naphthalenesulfonate, 4-toluenesulfonate, camphorate, camphorsulfonate, 4-methylbicyclo[2.2.2]-oct-2-ene-1-carboxylate, glucoheptonate, 3-phenylpropionate, trimethylacetate, tert-butylacetate, lauryl sulfate, gluconate, benzoate, glutamate, hydroxynaphthoate, salicylate, stearate, cyclohexylsulfamate, quinate, muconate and the like.

The term “substantially free of” or “substantially in the absence of,” when used in connection with an article (including, but not limited to, a compound, a salt thereof, a solvate thereof, a solid form thereof, and the like), refers to the article that includes at least 85% or 90% by weight, in certain embodiments, 95%, 98%, 99%, or 100% by weight, of the designated article. For example, the term “substantially free of” or “substantially in the absence of” with respect to a nucleoside composition can refer to a nucleoside composition that includes at least 85% or 90% by weight, in certain embodiments, 95%, 98%, 99%, or 100% by weight, of the designated enantiomer of that nucleoside. In certain embodiments, in the methods and compounds provided herein, the compounds are substantially free of undesignated enantiomers. For another example, the term “substantially free of” or “substantially in the absence of” with respect to a solid form can refer to a solid form that includes at least 85% or 90% by weight, in certain embodiments, 95%, 98%, 99%, or 100% by weight, of the designated solid form. In certain embodiments, in the methods and compounds provided herein, the solid form is substantially free of other solid forms.

As used herein and unless otherwise indicated, the term “stereomerically pure” means a composition that comprises one stereoisomer of a compound and is substantially free of other stereoisomers of that compound. In certain embodiments, a stereomerically pure compound comprises greater than about 80 percent by weight of one stereoisomer of the compound and less than about 20 percent by weight of other stereoisomers of the compound, greater than about 90 percent by weight of one stereoisomer of the compound and less than about 10 percent by weight of the other stereoisomers of the compound, greater than about 95 percent by weight of one stereoisomer of the compound and less than about 5 percent by weight of the other stereoisomers of the compound, greater than about 97 percent by weight of one stereoisomer of the compound and less than about 3 percent by weight of the other stereoisomers, or greater than about 99 percent by weight of one stereoisomer of the compound and less than about 1 percent by weight of the other stereoisomers of the compound. In certain embodiments, term “stereomerically pure” Compound I means that the compound is made up of approximately 100% by weight of this particular stereoisomer. The above percentages are based on the total amount of combined stereoisomers of the compound.

Similarly, the term “isolated” with respect to a nucleoside composition refers to a nucleoside composition that includes at least 85%, 90%, 95%, 98%, or 99% to 100% by weight, of the nucleoside, the remainder comprising other chemical species or enantiomers. Similarly, the term “isolated” with respect to a solid form of a compound refers to a solid that includes at least 85%, 90%, 95%, 98%, or 99% to 100% by weight, of such solid form of the compound, the remainder comprising other solid forms of the compound, other compounds, solvents, and/or other impurities.

The term “about” or “approximately” means an acceptable error for a particular value as determined by one of ordinary skill in the art, which depends in part on how the value is measured or determined. In certain embodiments, the term “about” or “approximately” means within 1, 2, 3, or 4 standard deviations. In certain embodiments, the term “about” or “approximately” means within 50%, 20%, 15%, 10%, 9%, 8%, 7%, 6%, 5%, 4%, 3%, 2%, 1%, 0.5%, or 0.05% of a given value or range.

“Solvate” refers to a compound provided herein, or a salt thereof, that further includes a stoichiometric or non-stoichiometric amount of solvent bound by non-covalent intermolecular forces. Where the solvent is water, the solvate is a hydrate.

The term “amorphous” or “amorphous form” is intended to mean that the substance, component, or product in question is not substantially crystalline as determined, for instance, by XRPD or where the substance, component, or product in question, for example is not birefringent when viewed microscopically. In certain embodiments, a sample comprising an amorphous form of a substance may be substantially free of other amorphous forms and/or crystalline forms.

The term “anti-solvent” refers to a liquid that is added to a solvent to reduce the solubility of a compound in that solvent, in some instances, resulting in precipitation of the compound.

The term “crystalline form” of a compound can refer to any crystalline form of the compound as a free acid, the compound as a free base, as an acid addition salt of the compound, a base addition salt of the compound, a complex of the compound, a solvate (including hydrate) of the compound, a clathrate of the compound, or a co-crystal of the compound. The term “solid form” of a compound can refer to any crystalline form of the compound or any amorphous form of the compound as a free acid, the compound as a free base, as an acid addition salt of the compound, an base addition salt of the compound, a complex of the compound, a clathrate of the compound, or a solvate (including hydrate) of the compound, or a co-precipitation of the compound. In many instances, the terms “crystalline form” and “solid form” can refer to those that are pharmaceutically acceptable, including, for example, those of pharmaceutically acceptable addition salts, pharmaceutically acceptable complexes, pharmaceutically acceptable solvates, a clathrate of the compound, pharmaceutically acceptable co-crystals, and pharmaceutically acceptable co-precipitations.

The term “polymorph” or “polymorphic form” refers to one of two or more crystal forms that comprise the same molecule, molecules or ions. Different polymorphs may have different physical properties such as, for example, melting temperatures, heats of fusion, solubilities, dissolution rates, and/or vibrational spectra as a result of the arrangement or conformation of the molecules or ions in the crystal lattice. The differences in physical properties exhibited by polymorphs may affect pharmaceutical parameters, such as storage stability, compressibility, density (important in formulation and product manufacturing), and dissolution rate (an important factor in bioavailability). Differences in stability can result from changes in chemical reactivity (e.g., differential oxidation, such that a dosage form discolors more rapidly when comprised of one polymorph than when comprised of another polymorph), mechanical changes (e.g., tablets crumble on storage as a kinetically favored polymorph converts to thermodynamically more stable polymorph), or both (e.g., tablets of one polymorph are more susceptible to breakdown at high humidity). In addition, the physical properties of a crystalline form may be important in processing; for example, one polymorph might be more likely to form solvates or might be difficult to filter and wash free of impurities (e.g., particle shape and size distribution might be different between polymorphs).

“Isotopic composition” refers to the amount of each isotope present for a given atom, and “natural isotopic composition” refers to the naturally occurring isotopic composition or abundance for a given atom. Atoms containing their natural isotopic composition may also be referred to herein as “non-enriched” atoms. Unless otherwise designated, the atoms of the compounds recited herein are meant to represent any stable isotope of that atom. For example, unless otherwise stated, when a position is designated specifically as “H” or “hydrogen,” the position is understood to have hydrogen at its natural isotopic composition.

“Isotopic enrichment” refers to the percentage of incorporation of an amount of a specific isotope at a given atom in a molecule in the place of that atom's natural isotopic abundance. For example, deuterium enrichment of 1% at a given position means that 1% of the molecules in a given sample contain deuterium at the specified position. Because the naturally occurring distribution of deuterium is about 0.0156%, deuterium enrichment at any position in a compound synthesized using non-enriched starting materials is about 0.0156%. The isotopic enrichment of the compounds provided herein can be determined using conventional analytical methods known to one of ordinary skill in the art, including mass spectrometry and nuclear magnetic resonance spectroscopy.

“Isotopically enriched” refers to an atom having an isotopic composition other than the natural isotopic composition of that atom. “Isotopically enriched” may also refer to a compound containing at least one atom having an isotopic composition other than the natural isotopic composition of that atom.

As used herein, EC₅₀ refers to a dosage, concentration, or amount of a particular test compound that elicits a dose-dependent response at 50% of maximal expression of a particular response that is induced, provoked, or potentiated by the particular test compound.

The term “host,” as used herein, refers to any unicellular or multicellular organism in which a virus can replicate, including cell lines and animals, and in certain embodiments, a human. In certain embodiments, the virus is a Flaviviridae virus, for example HCV. Alternatively, the host can be carrying a part of the Flaviviridae viral genome, whose replication or function can be altered by the compounds of the present invention. The term host specifically includes infected cells, cells transfected with all or part of the Flaviviridae genome and animals, in particular, primates (including chimpanzees) and humans. In most animal applications of the present invention, the host is a human patient. Veterinary applications, in certain indications, however, are clearly anticipated by the present invention (such as when the host is a chimpanzee).

As used herein, the terms “subject” and “patient” are used interchangeably herein. The terms “subject” and “subjects” refer to an animal, such as a mammal including a non-primate (e.g., a cow, pig, horse, cat, dog, rat, and mouse) and a primate (e.g., a monkey such as a cynomolgus monkey, a chimpanzee and a human), and for example, a human. In certain embodiments, the subject is refractory or non-responsive to current treatments for hepatitis C infection. In certain embodiments, the subject is a human.

As used herein, the terms “therapeutic agent” and “therapeutic agents” refer to any agent(s) which can be used in the treatment or prevention of a disorder or one or more symptoms thereof. In certain embodiments, the term “therapeutic agent” includes a compound provided herein. In certain embodiments, a therapeutic agent is an agent which is known to be useful for, or has been or is currently being used for the treatment or prevention of a disorder or one or more symptoms thereof.

“Therapeutically effective amount” refers to an amount of a compound or composition that, when administered to a subject for treating a disease, is sufficient to effect such treatment for the disease. A “therapeutically effective amount” can vary depending on, inter alia, the compound, the disease and its severity, and the age, weight, etc., of the subject to be treated.

“Treating” or “treatment” of any disease or disorder refers, in certain embodiments, to ameliorating a disease or disorder that exists in a subject. In another embodiment, “treating” or “treatment” includes ameliorating at least one physical parameter, which may be indiscernible by the subject. In yet another embodiment, “treating” or “treatment” includes modulating the disease or disorder, either physically (e.g., stabilization of a discernible symptom) or physiologically (e.g., stabilization of a physical parameter) or both. In yet another embodiment, “treating” or “treatment” includes delaying the onset of the disease or disorder.

As used herein, the terms “prophylactic agent” and “prophylactic agents” as used refer to any agent(s) which can be used in the prevention of a disorder or one or more symptoms thereof. In certain embodiments, the term “prophylactic agent” includes a compound provided herein. In certain other embodiments, the term “prophylactic agent” does not refer a compound provided herein. For example, a prophylactic agent is an agent which is known to be useful for, or has been or is currently being used to prevent or impede the onset, development, progression, and/or severity of a disorder.

As used herein, the phrase “prophylactically effective amount” refers to the amount of a therapy (e.g., prophylactic agent) which is sufficient to result in the prevention or reduction of the development, recurrence or onset of one or more symptoms associated with a disorder, or to enhance or improve the prophylactic effect(s) of another therapy (e.g., another prophylactic agent).

### Compound I Compositions

An aspect of the inventions features Compound I or a pharmaceutically acceptable salt thereof provided in an amount of about 25 to about 500 mg, and a pharmaceutically acceptable carrier. In different embodiments, Compound I or a pharmaceutically acceptable salt thereof is provided in an amount of: about 100 mg, 150 mg, 200 mg, 300 mg, 350 mg, 400 mg, 450 mg, and 500 mg. Example of Compound I compositions include capsule and tablets, and can be provided as different forms described herein. In some embodiments, the Compound I composition is provided as tablet, as a capsule, as a tablet containing Form I or as a capsule containing Form I.

### Solid Forms

Provided herein are solid forms of Compound I:

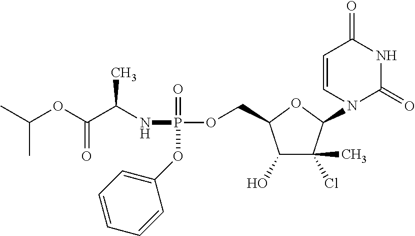 [SMILES: CC(C)OC(=O)[C@@H](C)N[P@@](=O)(OC[C@H]1O[C@@H](n2ccc(=O)[nH]c2=O)[C@](C)(Cl)[C@@H]1O)Oc1ccccc1]

(Compound I) or a pharmaceutical salt thereof.

Unless otherwise indicated, reference to a compound or pharmaceutically salt thereof includes different forms of the compound, such as polymorphs and solvates (including hydrates).

In various embodiments, provided herein are crystalline forms of Compound I. In some embodiments, a crystalline form of Compound I is Form I. In some embodiments, a crystalline form of Compound I is Form II. In some embodiments, a crystalline form of Compound I is Form III. In some embodiments, a crystalline form of Compound I is Form IV. In some embodiments, a crystalline form of Compound I is Form V. In some embodiments, a crystalline form of Compound I is Form VI. In some embodiments, a crystalline form of Compound I is Form VII. In some embodiments, a crystalline form of Compound I is Form VIII. In some embodiments, Form I, Form II, Form III, Form V, Form V, Form VI, Form VII, or Form VIII is in substantially pure crystalline form. In some embodiments, Form I, Form II, or Form II, Form IV, Form V, Form VI, Form VII, or Form VIII is substantially free of an amorphous form.

Crystalline forms can be characterized by a number of techniques. For example, by X-ray powder diffraction (XRPD). Another example is by differential scanning calorimetry (DSC). Other examples include by thermogravimetric analysis (TGA), vibrational spectroscopy, e.g., IR and Raman spectroscopy, solid-state NMR, optical microscopy, hot stage optical microscopy, scanning electron microscopy (SEM), electron crystallography and quantitative analysis, PSA, surface area analysis, solubility studies and dissolution studies.

In the embodiments, unless otherwise stated, the 2θ angle degrees value provided may vary to an extent of about ±0.2θ, while still describing the same XRPD peak. In another embodiment, unless otherwise stated, the 2θ angle degrees value provided may vary to an extent of about ±0.1θ, while still describing the same XRPD peak.

In an embodiment, Form I is characterized by an XRPD pattern. In some embodiments, the XRPD pattern of Form I includes an XRPD diffraction peak at a two-theta angle of about 6.8. In some embodiments, the XRPD pattern of Form I includes an XRPD diffraction peak at a two-theta angle of about 18.5. In some embodiments, the XRPD pattern of Form I includes an XRPD diffraction peak at a two-theta angle of about 15.7. In some embodiments, the XRPD pattern of Form I includes an XRPD diffraction peak at a two-theta angle of about 10.8. In some embodiments, the XRPD pattern of Form I includes an XRPD diffraction peak at a two-theta angle of about 18.0. In some embodiments, the XRPD pattern of Form I includes an XRPD diffraction peak at a two-theta angle of about 13.6. In some embodiments, the XRPD pattern of Form I includes an XRPD diffraction peak at a two-theta angle of about 8.2. In some embodiments, the XRPD pattern of Form I includes an XRPD diffraction peak at a two-theta angle of about 7.3. In some embodiments, the XRPD pattern of Form I includes an XRPD diffraction peak at a two-theta angle of about 19.3. In some embodiments, the XRPD pattern of Form I includes an XRPD diffraction peak at a two-theta angle of about 26.5. In some embodiments, the XRPD pattern of Form I includes an XRPD diffraction peak at a two-theta angle of about 23.6. In some embodiments, the XRPD pattern of Form I includes an XRPD diffraction peak at a two-theta angle of about 21.4. In some embodiments, the XRPD pattern of Form I includes an XRPD diffraction peak at a two-theta angle of about 19.7.

In some embodiments, the XRPD pattern of Form I includes XRPD diffraction peaks at two-theta angles of about 6.8 and about 18.5. In some embodiments, the XRPD pattern of Form I includes XRPD diffraction peaks at two-theta angles of about 6.8, about 18.5, and about 15.7. In some embodiments, the XRPD pattern of Form I includes XRPD diffraction peaks at two-theta angles of about 6.8, about 18.5, about 15.7, and about 10.8. In some embodiments, the XRPD pattern of Form I includes XRPD diffraction peaks at two-theta angles of about 6.8, about 18.5, about 15.7, about 10.8, and about 18.0. In some embodiments, the XRPD pattern of Form I includes XRPD diffraction peaks at two-theta angles of about 6.8, about 18.5, about 15.7, about 10.8, about 18.0, and about 13.6. In some embodiments, the XRPD pattern of Form I includes XRPD diffraction peaks at two-theta angles of about 6.8, about 18.5, about 15.7, about 10.8, about 18.0, about 13.6, and about 8.2. In some embodiments, the XRPD pattern of Form I includes XRPD diffraction peaks at two-theta angles of about 6.8, about 18.5, about 15.7, about 10.8, about 18.0, about 13.6, about 8.2, and about 7.3. In some embodiments, the XRPD pattern of Form I includes XRPD diffraction peaks at two-theta angles of about 6.8, about 18.5, about 15.7, about 10.8, about 18.0, about 13.6, about 8.2, about 7.3, and about 19.3. In some embodiments, the XRPD pattern of Form I includes XRPD diffraction peaks at two-theta angles of about 6.8, about 18.5, about 15.7, about 10.8, about 18.0, about 13.6, about 8.2, about 7.3, about 19.3, about 23.6, about 21.4, and about 19.7. In some embodiments, the XRPD pattern of Form I includes XRPD diffraction peaks at two-theta angles of about 6.8, about 18.5, about 15.7, about 10.8, about 18.0, about 13.6, about 8.2, about 7.3, about 19.3, about 26.5, about 21.4, and about 19.7. In some embodiments, the XRPD pattern of Form I includes XRPD diffraction peaks at two-theta angles of about 6.8, about 18.5, about 15.7, about 10.8, about 18.0, about 13.6, about 8.2, about 7.3, about 19.3, about 26.5, about 23.6, and about 19.7. In some embodiments, the XRPD pattern of Form I includes XRPD diffraction peaks at two-theta angles of about 6.8, about 18.5, about 15.7, about 10.8, about 18.0, about 13.6, about 8.2, about 7.3, about 19.3, about 26.5, about 23.6, and about 21.4. In some embodiments, the XRPD pattern of Form I includes XRPD diffraction peaks at two-theta angles of about 6.8, about 18.5, about 15.7, about 10.8, about 18.0, about 13.6, about 8.2, about 7.3, about 19.3, about 26.5, about 23.6, about 21.4, and about 19.7.

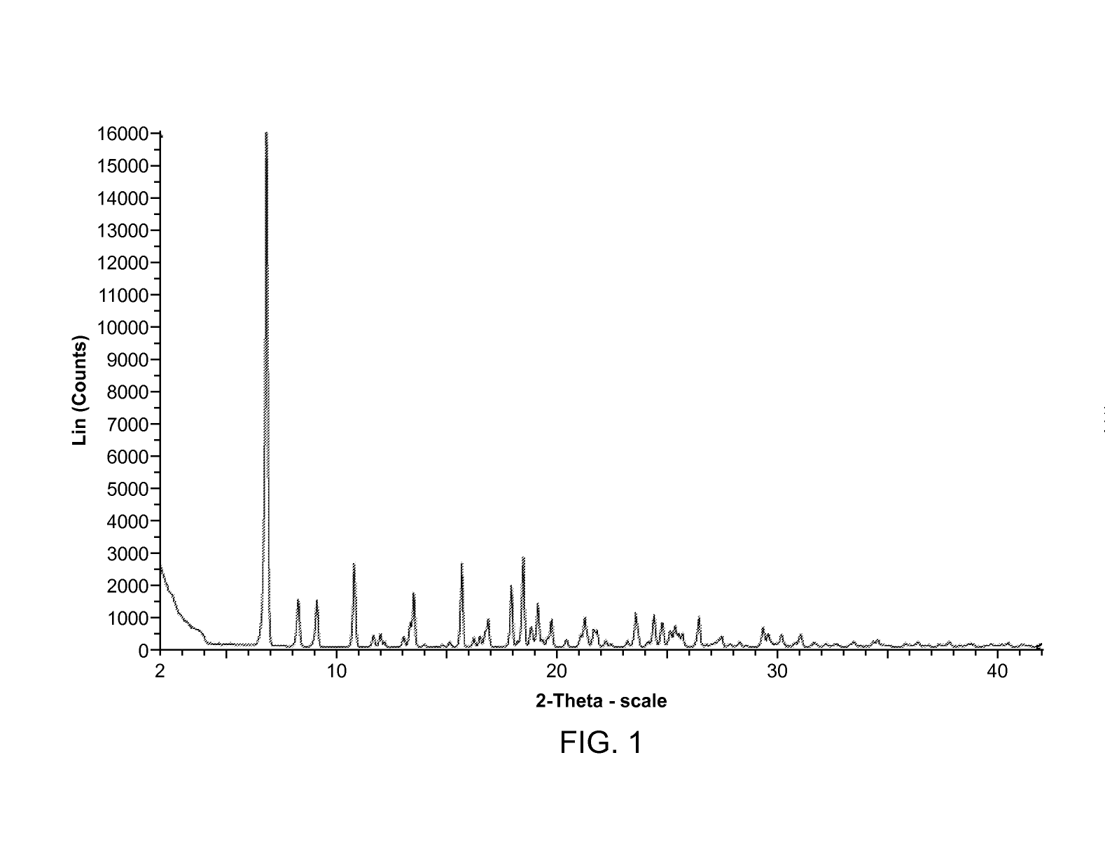

In particular embodiments, the XRPD pattern of Form I includes XRPD diffraction peaks at two-theta angles of about 6.8, about 7.3, about 8.2, about 10.8, about 13.6, about 15.7, about 16.8, about 18.0, about 18.5, about 19.3, about 19.7, about 21.4, about 21.8, about 23.6, about 24.4, about 24.8, and about 26.5. In one embodiment, the XRPD pattern of Form I is substantially as shown in FIG. 1.

In at least some of the embodiments where XRPD peaks are listed, the X-ray diffraction pattern is obtained using Cu Kα (40 kV/40 mA) radiation, the conditions of which are described in further detail herein.

In an embodiment, provided herein is Form I of Compound I, wherein 2θ angles of peaks as determined by X-ray powder diffraction using Cu Kα (40 kV/40 mA) radiation correspond to at least 2 of the above peaks and/or peaks in the XRPD of FIG. 1. In an embodiment, provided herein is Form I of Compound I, wherein 2θ angles of peaks as determined by X-ray powder diffraction using Cu Kα (40 kV/40 mA) radiation correspond to at least 3 of the above peaks and/or peaks in the XRPD of FIG. 1. In an embodiment, provided herein is Form I of Compound I, wherein 2θ angles of peaks as determined by X-ray powder diffraction using Cu Kα (40 kV/40 mA) radiation correspond to at least 4 of the above peaks and/or peaks in the XRPD of FIG. 1. In an embodiment, provided herein is Form I of Compound I, wherein 2θ angles of peaks as determined by X-ray powder diffraction using Cu Kα (40 kV/40 mA) radiation correspond to at least 5 of the above peaks and/or peaks in the XRPD of FIG. 1. In an embodiment, provided herein is Form I of Compound I, wherein 20 angles of peaks as determined by X-ray powder diffraction using Cu Kα (40 kV/40 mA) radiation correspond to at least 6 of the above peaks and/or peaks in the XRPD of FIG. 1. In an embodiment, provided herein is Form I of Compound I, wherein 2θ angles of peaks as determined by X-ray powder diffraction using Cu Kα (40 kV/40 mA) radiation correspond to at least 7 of the above peaks and/or peaks in the XRPD of FIG. 1. In an embodiment, provided herein is Form I of Compound I, wherein 2θ angles of peaks as determined by X-ray powder diffraction using Cu Kα (40 kV/40 mA) radiation correspond to at least 8 of the above peaks and/or peaks in the XRPD of FIG. 1. In an embodiment, provided herein is Form I of Compound I, wherein 2θ angles of peaks as determined by X-ray powder diffraction using Cu Kα (40 kV/40 mA) radiation correspond to at least 9 of the above peaks and/or peaks in the XRPD of FIG. 1. In an embodiment, provided herein is Form I of Compound I, wherein 20 angles of peaks as determined by X-ray powder diffraction using Cu Kα (40 kV/40 mA) radiation correspond to at least 10 of the above peaks and/or peaks in the XRPD of FIG. 1. In an embodiment, provided herein is Form I of Compound I, wherein 2θ angles of peaks as determined by X-ray powder diffraction using Cu Kα (40 kV/40 mA) radiation correspond to at least 11 of the above peaks and/or peaks in the XRPD of FIG. 1.

In further embodiments directed to X-ray powder diffraction obtained using Cu Kα (40 kV/40 mA) Form I of Compound I is characterized by X-ray powder diffraction comprising 2Θ angle of peaks of:
(a) about 6.8, about 15.7, and about 18.5, where in different embodiments reference to “about” for each angle indicates ±0.2, or ±0.1;
(b) about 6.8, about 15.7, about 18.5, about 10.8, about 13.6, and about 18.0, where in different embodiments reference to “about” for each angle indicates ±0.2, or ±0.1;
(c) about 6.8, about 15.7, about 18.5, about 10.8, about 13.6, about 18.0, about 7.3, about 8.2, and about 19.3, where in different embodiments reference to “about” for each angle indicates ±0.2, or ±0.1; and
(d) about 6.8, about 15.7, about 18.5, about 10.8, about 13.6, about 18.0, about 7.3, about 8.2, about 19.3, about 16.8, about 19.7, about 21.4, about 21.8, about 23.6, about 24.4, about 24.8 and about 26.5, where in different embodiments reference to “about” for each angle indicates ±0.2, or ±0.1.

In particular embodiments, the XRPD pattern of Form I includes one or more (e.g., at least one, at least two, at least three, at least four, at least five, at least six, at least seven, or at least eight) XRPD diffraction peaks selected from peaks at two-theta angles of about 6.8, about 7.3, about 8.2, about 10.8, about 13.6, about 15.7, about 16.8, about 18.0, about 18.5, about 19.3, about 19.7, about 21.4, about 21.8, about 23.6, about 24.4, about 24.8, and about 26.5.

In any of the ¹³C solid state NMR, the peak positions can vary depending on factors such as signal-to-noise ratio, peak width, temperature, spinning speed, decoupling efficiency, magic angle setting, data processing procedures and parameters, and software peak picking algorithm. In addition, peak position is relative to the chemical shift referencing procedure. Several different chemical shift reference standards may be used and will not necessarily give the same results. This may lead to peak positions that are different by several ppm. However, typically all of the peaks will have a systematic change in position in the same direction if a different reference standard was used or if the analyst used a different value for the reference peak position of the same standard. In some embodiments, the ppm values in the ¹³C solid state NMR provided herein may vary to an extent of about ±0.2 ppm, while still describing the same peak. In some embodiments, the ppm values in the ¹³C solid state NMR provided herein may vary to an extent of about ±0.1 ppm, while still describing the same peak.

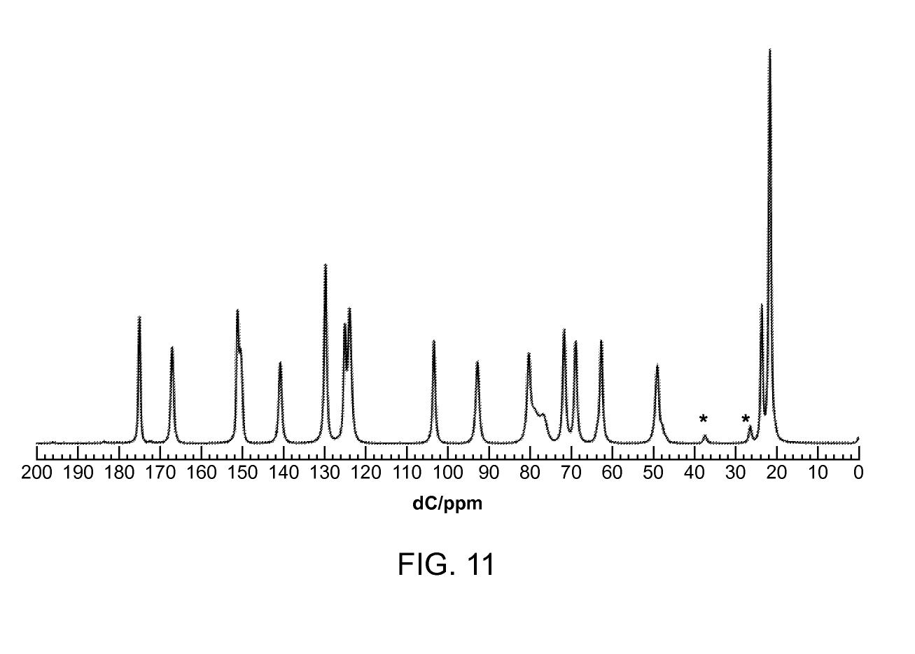

In certain embodiments, Form I exhibits a solid state ¹³C NMR spectrum corresponding substantially to the spectrum in FIG. 11. In certain embodiments, Form I exhibits a solid state ¹³C NMR spectrum with one or more peaks (e.g., at least three, at least four, at least five, at least six, or at least seven peaks) selected from about 175.2, about 167.3, about 151.4, about 150.7, about 141.0, about 130.0, about 125.2, about 124.1, about 103.5, about 93.0, about 80.5, about 77.1, about 71.9, about 69.1, about 62.9, about 49.2, about 23.8, and about 21.8. In certain embodiments, Form I exhibits a solid state ¹³C NMR spectrum with one or more peaks (e.g., at least three, at least four, at least five, at least six, or at least seven peaks) selected from about 175.2, about 167.3, about 151.4, about 130.0, about 125.2, about 124.1, about 103.5, about 71.9, about 69.1, about 62.9, about 23.8, and about 21.8. In some or any embodiments, Form I exhibits a solid state ¹³C NMR spectrum with one or more peaks (e.g., at least three, at least four, at least five, at least six, or at least seven peaks) selected from about 175.2, about 167.3, about 150.7, about 130.0, about 103.5, about 80.5, about 69.1, about 62.9, and about 49.2. In some or any embodiments, Form I exhibits a solid state ¹³C NMR spectrum with one or more peaks (e.g., at least three, at least four, or five) selected from about 167.3, about 150.7, about 80.5, about 69.1, and about 49.2.

Due to the present of different amounts of solution, Form I can have different differential scanning calorimetric thermogram values. In various embodiments, Form I has an endotherm in a differential scanning calorimetric thermogram. In some embodiments, Form I has an onset temperature above about 50° C. in a differential scanning calorimetric thermogram. In some embodiments, Form I has an onset temperature above about 70° C. in a differential scanning calorimetric thermogram. In some embodiments, Form I has an onset temperature above about 80° C. in a differential scanning calorimetric thermogram. In some embodiments, Form I has an onset temperature above about 90° C. in a differential scanning calorimetric thermogram. In some embodiments, Form I has an onset temperature above about 100° C. in a differential scanning calorimetric thermogram. In some embodiments, Form I has an onset temperature above about 105° C. in a differential scanning calorimetric thermogram.

In certain embodiments, Form I has an onset temperature between about 50° C. to about 200° C. in a differential scanning calorimetric thermogram. In certain embodiments, Form I has an onset temperature between about 70° C. to about 180° C. in a differential scanning calorimetric thermogram. In certain embodiments, Form I has an onset temperature between about 80° C. to about 160° C. in a differential scanning calorimetric thermogram. In certain embodiments, Form I has an onset temperature between about 90° C. to about 150° C. in a differential scanning calorimetric thermogram. In certain embodiments, Form I has an onset temperature between about 100° C. to about 140° C. in a differential scanning calorimetric thermogram. In certain embodiments, Form I has an onset temperature between about 105° C. to about 120° C. in a differential scanning calorimetric thermogram. In certain embodiments, Form I has an onset temperature between about 110° C. to about 120° C. in a differential scanning calorimetric thermogram.

In certain embodiments, Form I has an onset temperature of about 110° C. in a differential scanning calorimetric thermogram. In certain embodiments, Form I has an onset temperature of about 111° C. in a differential scanning calorimetric thermogram. In certain embodiments, Form I has an onset temperature of about 112° C. in a differential scanning calorimetric thermogram. In certain embodiments, Form I has an onset temperature of about 113° C. in a differential scanning calorimetric thermogram. In certain embodiments, Form I has an onset temperature of about 114° C. in a differential scanning calorimetric thermogram. In certain embodiments, Form I has an onset temperature of about 115° C. in a differential scanning calorimetric thermogram. In certain embodiments, Form I has an onset temperature of about 116° C. in a differential scanning calorimetric thermogram. In certain embodiments, Form I has an onset temperature of about 117° C. in a differential scanning calorimetric thermogram.

In some embodiments, Form I has a peak temperature above about 50° C. in a differential scanning calorimetric thermogram. In some embodiments, Form I has a peak temperature above about 70° C. in a differential scanning calorimetric thermogram. In some embodiments, Form I has a peak temperature above about 80° C. in a differential scanning calorimetric thermogram. In some embodiments, Form I has a peak temperature above about 90° C. in a differential scanning calorimetric thermogram. In some embodiments, Form I has a peak temperature above about 100° C. in a differential scanning calorimetric thermogram. In some embodiments, Form I has a peak temperature above about 105° C. in a differential scanning calorimetric thermogram. In some embodiments, Form I has a peak temperature above about 110° C. in a differential scanning calorimetric thermogram.

In some embodiments, Form I has a peak temperature between about 50° C. and about 300° C. in a differential scanning calorimetric thermogram. In some embodiments, Form I has a peak temperature between about 70° C. and about 280° C. in a differential scanning calorimetric thermogram. In some embodiments, Form I has a peak temperature between about 80° C. and about 240° C. in a differential scanning calorimetric thermogram. In some embodiments, Form I has a peak temperature between about 90° C. and about 200° C. in a differential scanning calorimetric thermogram. In some embodiments, Form I has a peak temperature between about 100° C. and about 160° C. in a differential scanning calorimetric thermogram. In some embodiments, Form I has a peak temperature between about 105° C. and about 140° C. in a differential scanning calorimetric thermogram. In some embodiments, Form I has a peak temperature between about 110° C. and about 130° C. in a differential scanning calorimetric thermogram.

In certain embodiments, Form I has an endotherm with a peak temperature of about 114° C., in another embodiment of about 115° C., in another embodiment of about 116° C., in another embodiment of about 117° C., in another embodiment of about 118° C., in another embodiment of about 119° C., in another embodiment of about 120° C., in a differential scanning calorimetric thermogram. In certain embodiments, Form I has an endotherm with an onset temperature of about 113° C., in another embodiment of about 114° C., in another embodiment of about 115° C., in another embodiment of about 116° C., in another embodiment of about 117° C., in a differential scanning calorimetric thermogram.

In certain embodiments, Form I exhibits at least one of
an X-ray powder diffraction pattern comprising at least three (in certain embodiments, three, four, five, six, seven, eight, or nine) peaks at 2θ angle degrees±0.2 2θ angle degrees of about 6.8, about 7.3, about 8.2, about 10.8, about 13.6, about 15.7, about 18.0, about 18.5, and about 19.3;
a differential scanning calorimetry thermogram having an endotherm with a maximum at between about 110° C. and about 120° C.; and
a solid state ¹³C NMR spectrum with at least three (in certain embodiments, three, four, five, six, seven, eight, or nine) peaks at about 175.2, about 167.3, about 150.7, about 130.0, about 103.5, about 80.5, about 69.1, about 62.9, and about 49.2.

A variety of different solvates can be produced from Compound I. In certain embodiments, provided herein is Form II of Compound I. In certain embodiments, Form II comprises acetone. For example, Form II can be a solvate. In certain embodiments, Form II comprises an acetone solvate. In certain embodiments, Form II has unit cell dimensions of a=about 15.13 Å, b=about 15.13 Å, c=about 24.65 Å, and α=β=γ=90° according to a single crystal X-ray diffraction experiment. In certain embodiments, Form II has a density of about 1.35 g/cm³ according to a single crystal X-ray diffraction experiment. In certain embodiments, Form II is capable of conversion to Form I. Without being limited to a particular theory, Form II can be a mixture of Form I and a solvent, for example, that is trapped in the Form I crystal structure. In certain embodiments, Form II includes a solvent other than acetone, including cyclohexane, 1,4-dioxane, heptanes, or a mixture thereof.

In certain embodiments, provided herein is Form III of Compound I. In certain embodiments, Form III comprises 1-propanol. For example, Form III can be a solvate of Compound I. In certain embodiments, Form III comprises an isopropanol solvate. In certain embodiments, Form III has unit cell dimensions of a=about 15.13 Å, b=about 15.13 Å, and c=about 24.71 Å, and α=β=γ=90° according to a single crystal X-ray diffraction experiment. In certain embodiments, Form II has a density of about 1.31 g/cm³ according to a single crystal X-ray diffraction experiment. In certain embodiments, Form III is capable of conversion to Form I. Without being limited to a particular theory, Form III can be a mixture of Form I and a solvent, for example, that is trapped in the Form I crystal structure. In certain embodiments, Form II includes a solvent other than acetone, including propyl acetate, isopropyl acetate, nitromethane, or a mixture thereof.

In certain embodiments, provided herein is amorphous Compound I. Amorphous Compound I can be prepared according to techniques apparent to those of skill in the art, including those described below. In certain embodiments, amorphous Compound I has a glass transition temperature at about 63° C. In certain embodiments, amorphous Compound I has a kinetic solubility of about 2.0 mg/mL. In certain embodiments, amorphous Compound I is a fluffy white solid. In certain embodiments, amorphous Compound I has an endotherm onset at about 57° C. in a differential scanning calorimetric thermogram. In certain embodiments, amorphous Compound I has an exotherm onset at about 184° C. in a differential scanning calorimetric thermogram.

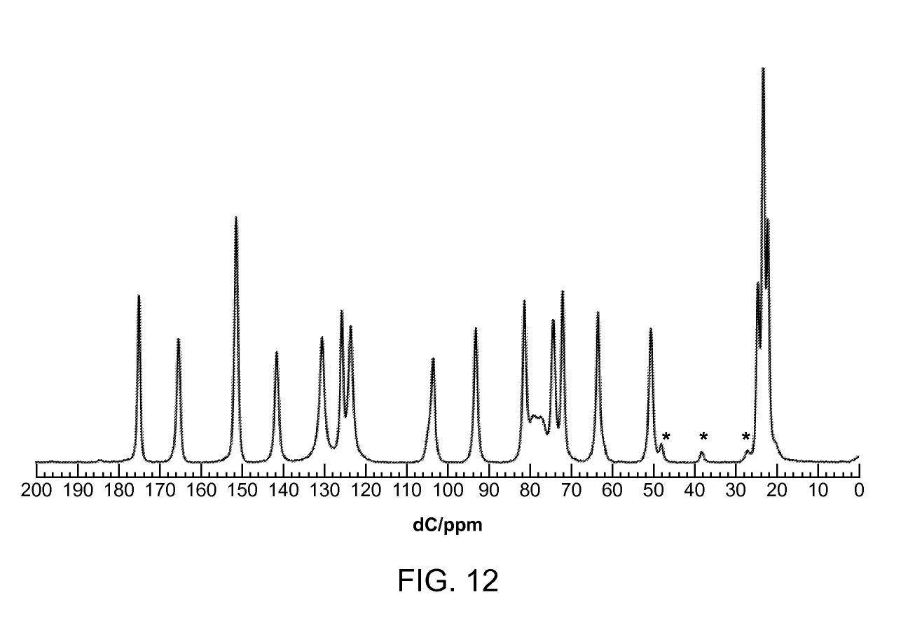

In certain embodiments, Form IV exhibits a solid state ¹³C NMR spectrum corresponding substantially to the spectrum in FIG. 12. In certain embodiments, Form IV exhibits a solid state ¹³C NMR spectrum with one or more peaks (e.g., at least three, at least four, at least five, at least six, or at least seven peaks) selected from about 175.1, about 165.5, about 151.5, about 141.7, about 130.6, about 125.8, about 123.7, about 103.6, about 93.2, about 81.4, about 79.1, about 77.4, about 74.4, about 72.1, about 63.5, about 50.6, about 24.6, about 23.3, and about 22.3. In certain embodiments, Form IV exhibits a solid state ¹³C NMR spectrum with one or more peaks (e.g., at least three, at least four, at least five, at least six, or at least seven peaks) selected from about 175.11, about 165.5, about 151.5, about 125.8, about 81.4, about 79.1, about 74.4, about 72.1, about 63.5, about 50.6, about 24.6, about 23.3, and about 22.3. In certain embodiments, Form IV exhibits a solid state ¹³C NMR spectrum with one or more peaks (e.g., at least three, at least four, at least five, or six peaks) selected from about 165.5, about 81.4, about 79.1, about 74.4, about 50.6, and about 24.6.

In an embodiment, Form IV is characterized by an XRPD pattern. In some embodiments, the XRPD pattern of Form IV includes an XRPD diffraction peak at a two-theta angle of about 5.7. In an embodiment, Form IV is characterized by an XRPD pattern. In some embodiments, the XRPD pattern of Form IV includes an XRPD diffraction peak at a two-theta angle of about 9.6. In some embodiments, the XRPD pattern of Form IV includes an XRPD diffraction peak at a two-theta angle of about 10.4. In some embodiments, the XRPD pattern of Form IV includes an XRPD diffraction peak at a two-theta angle of about 11.4. In some embodiments, the XRPD pattern of Form IV includes an XRPD diffraction peak at a two-theta angle of about 13.5. In some embodiments, the XRPD pattern of Form IV includes an XRPD diffraction peak at a two-theta angle of about 13.8. In some embodiments, the XRPD pattern of Form IV includes an XRPD diffraction peak at a two-theta angle of about 14.6. In some embodiments, the XRPD pattern of Form IV includes an XRPD diffraction peak at a two-theta angle of about 15.2. In some embodiments, the XRPD pattern of Form IV includes an XRPD diffraction peak at a two-theta angle of about 17.1. In some embodiments, the XRPD pattern of Form IV includes an XRPD diffraction peak at a two-theta angle of about 17.7. In some embodiments, the XRPD pattern of Form IV includes an XRPD diffraction peak at a two-theta angle of about 18.3. In some embodiments, the XRPD pattern of Form IV includes an XRPD diffraction peak at a two-theta angle of about 19.7. In some embodiments, the XRPD pattern of Form IV includes an XRPD diffraction peak at a two-theta angle of about 20.3. In some embodiments, the XRPD pattern of Form IV includes an XRPD diffraction peak at a two-theta angle of about 20.9. In some embodiments, the XRPD pattern of Form IV includes an XRPD diffraction peak at a two-theta angle of about 21.1. In some embodiments, the XRPD pattern of Form IV includes an XRPD diffraction peak at a two-theta angle of about 21.6. In some embodiments, the XRPD pattern of Form IV includes an XRPD diffraction peak at a two-theta angle of about 22.2. In some embodiments, the XRPD pattern of Form IV includes an XRPD diffraction peak at a two-theta angle of about 22.8. In some embodiments, the XRPD pattern of Form IV includes an XRPD diffraction peak at a two-theta angle of about 23.7. In some embodiments, the XRPD pattern of Form IV includes an XRPD diffraction peak at a two-theta angle of about 24.3. In some embodiments, the XRPD pattern of Form IV includes an XRPD diffraction peak at a two-theta angle of about 24.9. In some embodiments, the XRPD pattern of Form IV includes an XRPD diffraction peak at a two-theta angle of about 25.4. In some embodiments, the XRPD pattern of Form IV includes an XRPD diffraction peak at a two-theta angle of about 26.4. In some embodiments, the XRPD pattern of Form IV includes an XRPD diffraction peak at a two-theta angle of about 27.2. In some embodiments, the XRPD pattern of Form IV includes an XRPD diffraction peak at a two-theta angle of about 27.8. In some embodiments, the XRPD pattern of Form IV includes an XRPD diffraction peak at a two-theta angle of about 28.5. In some embodiments, the XRPD pattern of Form IV includes an XRPD diffraction peak at a two-theta angle of about 29.3. In some embodiments, the XRPD pattern of Form IV includes an XRPD diffraction peak at a two-theta angle of about 30.0. In some embodiments, the XRPD pattern of Form IV includes an XRPD diffraction peak at a two-theta angle of about 30.8. In some embodiments, the XRPD pattern of Form IV includes an XRPD diffraction peak at a two-theta angle of about 31.3. In some embodiments, the XRPD pattern of Form IV includes an XRPD diffraction peak at a two-theta angle of about 32.0. In some embodiments, the XRPD pattern of Form IV includes an XRPD diffraction peak at a two-theta angle of about 32.8. In some embodiments, the XRPD pattern of Form IV includes an XRPD diffraction peak at a two-theta angle of about 33.7. In some embodiments, the XRPD pattern of Form IV includes an XRPD diffraction peak at a two-theta angle of about 34.8. In some embodiments, the XRPD pattern of Form IV includes an XRPD diffraction peak at a two-theta angle of about 36.2. In some embodiments, the XRPD pattern of Form IV includes an XRPD diffraction peak at a two-theta angle of about 37.6. In some embodiments, the XRPD pattern of Form IV includes an XRPD diffraction peak at a two-theta angle of about 39.5. In some embodiments, the XRPD pattern of Form IV includes an XRPD diffraction peak at a two-theta angle of about 40.3.

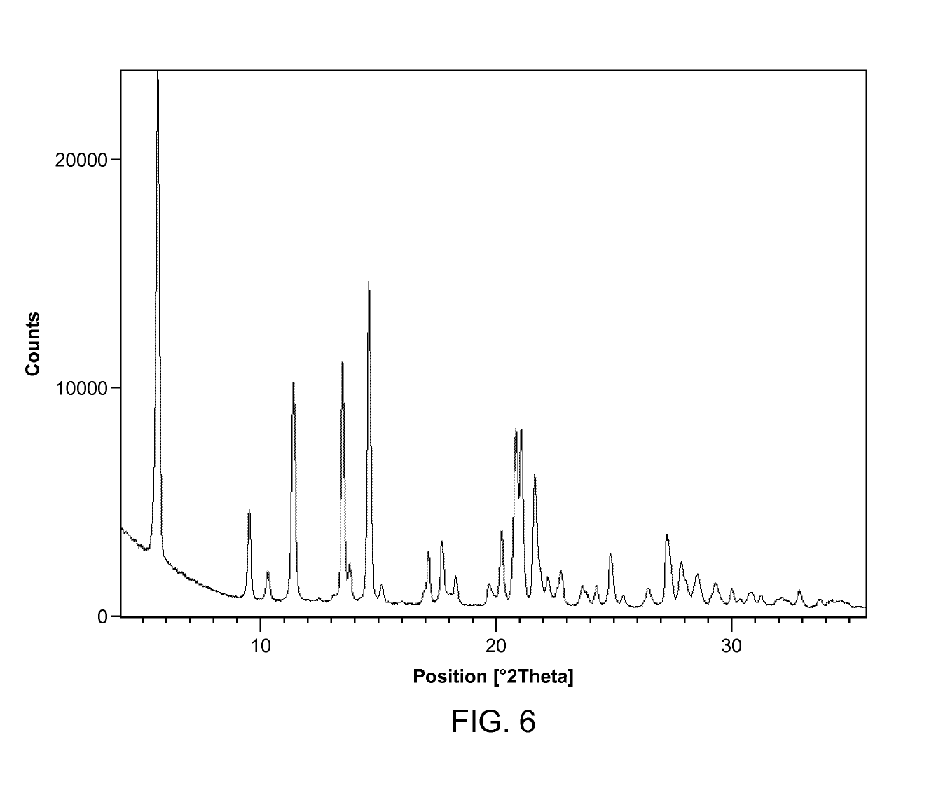

In particular embodiments, the XRPD pattern of Form IV includes one or more (e.g. at least one, at least two, at least three, at least four, at least six, at least seven, or at least eight) XRPD diffraction peaks selected from peaks at two-theta angles of about 5.7, about 9.6, about 10.4, about 11.4, about 13.5, about 13.8, about 14.6, about 15.2, about 17.1, about 17.7, about 18.3, about 19.7, about 20.3, about 20.9, about 21, about 21.6, about 22.2, about 22.8, about 23.7, about 24.3, about 24.9, about 25.4, about 26.4, about 27.2, about 27.8, about 28.5, about 29.3, about 30.0, about 30.8, about 31.3, about 32.0, about 32.8, about 33.7, about 34.8, about 36.2, about 37.6, about 39.5, and about 40.3. In one embodiment, the XRPD pattern of Form IV is substantially as shown in FIG. 6. In particular embodiments, the XRPD pattern of Form IV includes one or more (e.g., at least one, at least two, at least three, at least four, at least five, at least six, at least seven, or at least eight) XRPD diffraction peaks selected from peaks at two-theta angles of about 5.7, about 9.6, about 11.4, about 13.5, about 14.6, about 17.1, about 17.7, about 20.3, about 20.9, about 21.1, about 21.6, about 24.9, and about 27.2.

In further embodiments directed to X-ray powder diffraction obtained using Cu Kα (40 kV/40 mA) Form I of Compound I is characterized by X-ray powder diffraction comprising 2Θ angle of peaks of:
(a) about 5.7, about 13.5, and about 14.6, where in different embodiments reference to “about” for each angle indicates ±0.2, or ±0.1;
(b) about 5.7, about 11.4, about 13.5, about 14.6, about 20.9, about 21.1, and about 21.6, where in different embodiments reference to “about” for each angle indicates ±0.2, or ±0.1;
(c) about 5.7, about 9.6, about 11.4, about 13.5, about 14.6, about 20.3, about 20.9, about 21.1, and about 21.6, where in different embodiments reference to “about” for each angle indicates ±0.2, or ±0.1; and
(d) about 5.7, about 11.4, about 13.5, about 14.6, about 17.1, about 17.7, about 20.3, about 20.9, about 21.1, about 21.6, about 24.9, and about 27.2, where in different embodiments reference to “about” for each angle indicates ±0.2, or ±0.1.

In certain embodiments, Form IV exhibits at least one of
an X-ray powder diffraction pattern comprising at least three (in certain embodiments, three, four, five, six, seven, eight, or nine) peaks at 2θ angle degrees±0.2 2θ angle degrees of about 5.7, about 11.4, about 13.5, about 14.6, about 20.9, about 21.1, and about 21.6; and
a solid state ¹³C NMR spectrum with at least three (in certain embodiments, three, four, five, six, seven, eight, or nine) peaks at about 165.5, about 81.4, about 79.1, about 74.4, about 50.6, and about 24.6.

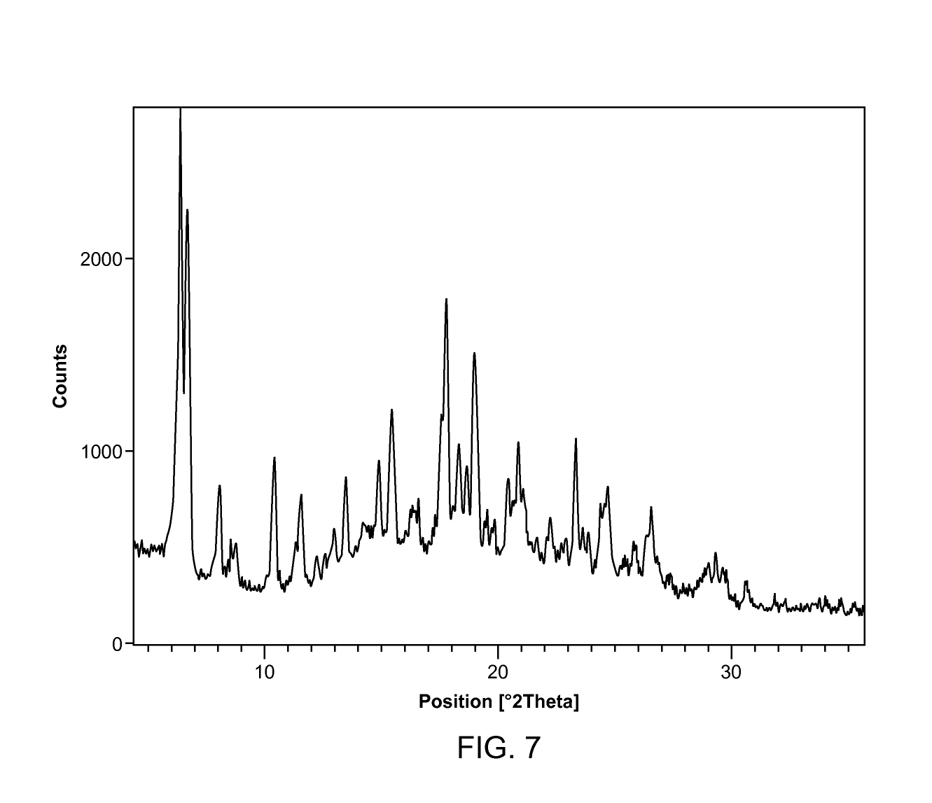

In one embodiment, the XRPD pattern of Form V is substantially as shown in FIG. 7. In particular embodiments, the XRPD pattern of Form V includes one or more (e.g., at least one, at least two, at least three, at least four, at least five, at least six, at least seven, or at least eight) XRPD diffraction peaks selected from peaks at two-theta angles of about 6.4, about 6.7, about 8.0, about 8.8, about 10.4, about 11.6, about 13.0, about 13.5, about 14.9, about 15.5, about 16.6, about 17.8, about 18.3, about 18.7, about 19.0, about 19.5, about 20.4, about 20.9, about 22.2, about 23.3, about 23.9, about 24.4, about 24.7, about 25.8, about 26.6, about 27.4, about 29.3, about 29.7, and about 30.6. In particular embodiments, the XRPD pattern of Form V includes one or more (e.g., at least one, at least two, at least three, at least four, at least five, at least six, at least seven, or at least eight) XRPD diffraction peaks selected from peaks at two-theta angles of about 6.4, about 6.7, about 8.0, about 10.4, about 11.6, about 13.5, about 14.9, about 15.5, about 16.6, about 17.8, about 18.3, about 18.7, about 19.0, about 19.5, about 20.4, about 20.9, about 22.2, about 23.3, about 23.9, about 24.4, about 24.7, and about 26.6. In particular embodiments, the XRPD pattern of Form V includes one or more (e.g., at least one, at least two, at least three, at least four, at least five, at least six, at least seven, or at least eight) XRPD diffraction peaks selected from peaks at two-theta angles of about 6.4, about 6.7, about 14.9, about 15.5, about 17.8, about 18.3, about 18.7, about 19.0, about 20.9, and about 23.3.

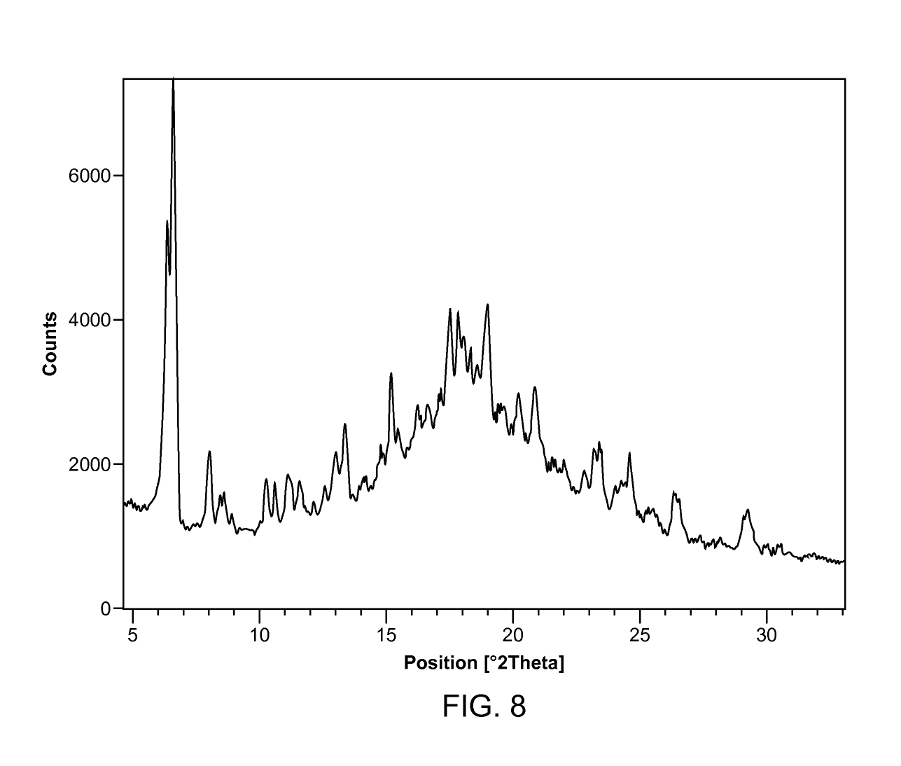

In one embodiment, the XRPD pattern of Form VI is substantially as shown in FIG. 8. In particular embodiments, the XRPD pattern of Form VI includes one or more (e.g., at least one, at least two, at least three, at least four, at least five, at least six, at least seven, or at least eight) XRPD diffraction peaks selected from peaks at two-theta angles of about 6.4, about 6.6, about 8.1, about 8.6, about 10.3, about 10.7, about 11.1, about 11.6, about 13.1, about 13.4, about 14.8, about 15.2, about 16.3, about 16.7, about 17.5, about 17.8, about 19.0, about 19.6, about 20.2, about 20.9, about 23.5, about 24.6, about 25.6, about 26.3, about 29.2, and about 30.5. In particular embodiments, the XRPD pattern of Form VI includes one or more (e.g., at least one, at least two, at least three, at least four, at least five, at least six, at least seven, or at least eight) XRPD diffraction peaks selected from peaks at two-theta angles of about 6.4, about 6.6, about 8.1, about 13.1, about 13.4, about 14.8, about 15.2, about 16.3, about 16.7, about 17.5, about 17.8, about 19.0, about 19.6, about 20.2, about 20.9, about 23.5, about 24.6, and about 26.3. In particular embodiments, the XRPD pattern of Form VI includes one or more (e.g., at least one, at least two, at least three, at least four, at least five, at least six, at least seven, or at least eight) XRPD diffraction peaks selected from peaks at two-theta angles of about 6.4, about 6.6, about 15.2, about 16.3, about 16.7, about 17.5, about 17.8, about 19.0, about 19.6, about 20.2, and about 20.9.

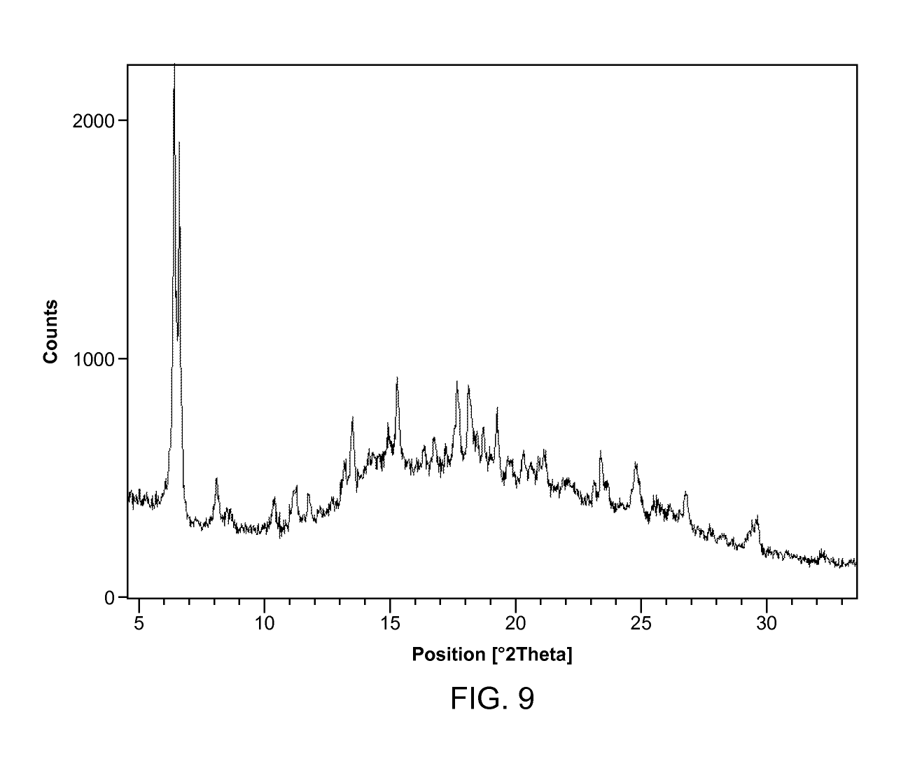

In one embodiment, the XRPD pattern of Form VII is substantially as shown in FIG. 9. In particular embodiments, the XRPD pattern of Form VII includes one or more (e.g., at least one, at least two, at least three, at least four, at least five, at least six, at least seven, or at least eight) XRPD diffraction peaks selected from peaks at two-theta angles of about 6.4, about 6.6, about 8.1, about 10.4, about 11.3, about 13.5, about 15.3, about 16.8, about 17.7, about 18.1, about 19.3, about 21.2, about 23.4, about 24.8, about 26.8, and about 29.6. In particular embodiments, the XRPD pattern of Form VII includes one or more (e.g., at least one, at least two, at least three, at least four, at least five, at least six, at least seven, or at least eight) XRPD diffraction peaks selected from peaks at two-theta angles of about 6.4, about 6.6, about 13.5, about 15.3, about 16.8, about 17.7, about 18.1, about 19.3, about 21.2, about 23.4, and about 24.8. In particular embodiments, the XRPD pattern of Form VII includes one or more (e.g., at least one, at least two, at least three, at least four, at least five, at least six, or seven) XRPD diffraction peaks selected from peaks at two-theta angles of about 6.4, about 6.6, about 13.5, about 15.3, about 17.7, about 18.1, and about 19.3.

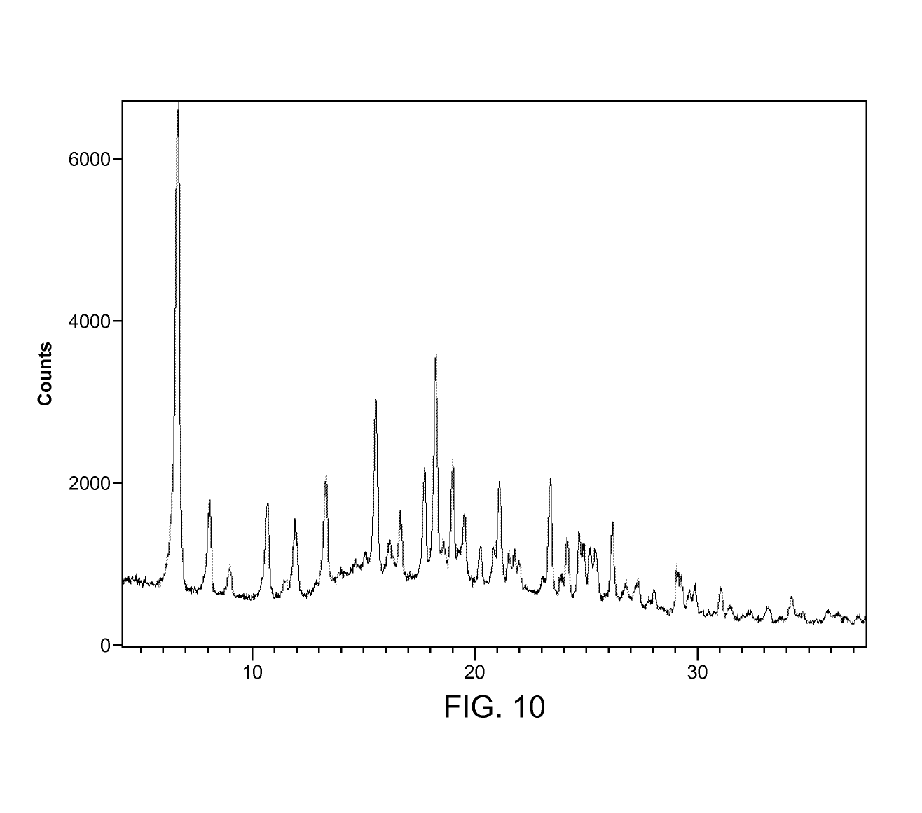

In one embodiment, the XRPD pattern of Form VIII is substantially as shown in FIG. 10. In particular embodiments, the XRPD pattern of Form VIII includes one or more (e.g., at least one, at least two, at least three, at least four, at least five, at least six, at least seven, or at least eight) XRPD diffraction peaks selected from peaks at two-theta angles of about 6.8, about 8.2, about 9.1, about 10.8, about 12.0, about 13.4, about 15.6, about 16.2, about 16.7, about 17.8, about 18.3, about 18.6, about 19.1, about 19.6, about 20.3, about 21.1, about 21.5, about 21.8, about 22.0, about 23.4, about 24.2, about 24.7, about 24.9, about 25.2, about 25. 5, and about 26.2. In particular embodiments, the XRPD pattern of Form VIII includes one or more (e.g., at least one, at least two, at least three, at least four, at least five, at least six, at least seven, or at least eight) XRPD diffraction peaks selected from peaks at two-theta angles of about 6.8, about 8.2, about 10.8, about 12.0, about 13.4, about 15.6, about 16.7, about 17.8, about 18.3, about 19.1, about 19.6, about 21.1, about 23.4, about 24.2, about 24.7, and about 26.2. In particular embodiments, the XRPD pattern of Form VIII includes one or more (e.g., at least one, at least two, at least three, at least four, at least five, at least six, at least seven, or at least eight) XRPD diffraction peaks selected from peaks at two-theta angles of about 6.8, about 15.6, about 17.8, about 18.3, about 19.1, about 21.1, about 23.4, and about 26.2.

In some embodiments, provided herein are:
(a) solid forms as described herein, e.g., crystalline forms of Compound I (including Forms I, II, III, IV, V, VI, VII, and VIII) and pharmaceutically acceptable salts and compositions thereof;
(b) solid forms as described herein, e.g., crystalline forms of Compound I including Forms I, II, III, IV, V, VI, VII, and VIII), and pharmaceutically acceptable salts and compositions thereof for use in the treatment and/or prophylaxis of a liver disorder including Flaviviridae infection, especially in individuals diagnosed as having a Flaviviridae infection or being at risk of becoming infected by hepatitis C;
(c) processes for the preparation of solid forms as described herein, e.g., crystalline forms of Compound I (including Forms I, II, III, IV, V, VI, VII, and VIII), as described in more detail elsewhere herein;
(d) pharmaceutical formulations comprising a solid form as described herein, e.g., a crystalline form of Compound I (including Form I, II, III, IV, V, VI, VII, and VIII), or a pharmaceutically acceptable salt thereof together with a pharmaceutically acceptable carrier;
(e) pharmaceutical formulations comprising a solid form as described herein, e.g., a crystalline form of Compound I (including Form I, II, III, IV, V, VI, VII, and VIII), or a pharmaceutically acceptable salt thereof together with one or more other effective anti-HCV agents, optionally in a pharmaceutically acceptable carrier;
(f) a method for the treatment and/or prophylaxis of a host infected with Flaviviridae that includes the administration of an effective amount of a compound as described herein, e.g., a solid form of Compound I (including crystalline forms, such as Form I, II, III, IV, V, VI, VII, and VIII), its pharmaceutically acceptable salt or composition; and
(g) a method for the treatment and/or prophylaxis of a host infected with Flaviviridae that includes the administration of an effective amount of a compound as described herein, e.g., a solid form of Compound I (including crystalline forms, such as Form I, II, III, IV, V, VI, VII, and VIII), its pharmaceutically acceptable salt or composition in combination and/or alternation with one or more effective anti-HCV agent.

In another embodiment, Form I, Form II, III, IV, V, VI, VII, or VIII is substantially pure. Reference to “substantially pure” with respect to a particular form means the form makes up at least 50% of that compound (e.g., Compound I) present. In different embodiments, a particular form makes up at least 75%, at least 85%, at least 90%, at least 95%, or about 94%-98% of compound (e.g., Compound I) present.

Diastereomeric Compounds

It is appreciated that compounds provided herein have several chiral centers and may exist in, and be isolated in, diastereomeric forms and mixtures thereof. It being well known in the art how to prepare diastereomeric forms (for example, by resolution of the racemic form by recrystallization techniques, by synthesis from diastereomeric starting materials, by chiral synthesis, or by chromatographic separation using a chiral stationary phase).

Examples of methods to obtain stereomerically pure materials are known in the art, and include at least the following.
i) physical separation of crystals—a technique whereby macroscopic crystals of the individual stereoisomers are manually separated. This technique can be used if crystals of the separate stereoisomers exist, i.e., the material is a conglomerate, and the crystals are visually distinct;
ii) simultaneous crystallization—a technique whereby the individual stereoisomers are separately crystallized from a solution of the racemate, possible only if the latter is a conglomerate in the solid state;
iii) enzymatic resolutions—a technique whereby partial or complete separation of a racemate by virtue of differing rates of reaction for the stereoisomers with an enzyme;
iv) enzymatic asymmetric synthesis—a synthetic technique whereby at least one step of the synthesis uses an enzymatic reaction to obtain a stereomerically pure or enriched synthetic precursor of the desired stereoisomer;
v) chemical asymmetric synthesis—a synthetic technique whereby the desired enantiomer or diastereomer is synthesized from an achiral precursor under conditions that produce asymmetry (i.e., chirality) in the product, which may be achieved using chiral catalysts or chiral auxiliaries;
vi) diastereomer separations—a technique whereby a racemic compound is reacted with an enantiomerically pure reagent (the chiral auxiliary) that converts the individual isomers to diastereomers. The resulting diastereomers are then separated by chromatography or crystallization by virtue of their now more distinct structural differences and the chiral auxiliary later removed to obtain the desired isomer;
vii) first- and second-order asymmetric transformations—a technique whereby diastereomers from the racemate equilibrate to yield a preponderance in solution of the diastereomer from the desired isomer or where preferential crystallization of the diastereomer from the desired isomer perturbs the equilibrium such that eventually in principle all the material is converted to the crystalline diastereomer from the desired isomer. The desired enantiomer is then released from the diastereomer;
viii) kinetic resolutions—this technique refers to the achievement of partial or complete resolution of a racemate (or of a further resolution of a partially resolved compound) by virtue of unequal reaction rates of the stereoisomers with a chiral, non-racemic reagent or catalyst under kinetic conditions;
ix) enantiospecific synthesis from non-racemic precursors—a synthetic technique whereby the desired enantiomer is obtained from non-chiral starting materials and where the stereochemical integrity is not or is only minimally compromised over the course of the synthesis;
x) chiral liquid chromatography—a technique whereby the stereoisomers of a racemate are separated in a liquid mobile phase by virtue of their differing interactions with a stationary phase. The stationary phase can be made of chiral material or the mobile phase can contain an additional chiral material to provoke the differing interactions;
xi) chiral gas chromatography—a technique whereby the racemate is volatilized and stereoisomers are separated by virtue of their differing interactions in the gaseous mobile phase with a column containing a fixed non-racemic chiral adsorbent phase;
xii) extraction with chiral solvents—a technique whereby the stereoisomers are separated by virtue of preferential dissolution of one stereoisomer into a particular chiral solvent;
xiii) transport across chiral membranes—a technique whereby a racemate is placed in contact with a thin membrane barrier. The barrier typically separates two miscible fluids, one containing the racemate, and a driving force such as concentration or pressure differential causes preferential transport across the membrane barrier. Separation occurs as a result of the non-racemic chiral nature of the membrane which allows only one stereoisomer of the racemate to pass through.

Isotopically Enriched Compounds

The atoms in a compound described herein may exhibit their natural isotopic abundances, or one or more of the atoms may be artificially enriched in a particular isotope having the same atomic number, but an atomic mass or mass number different from the atomic mass or mass number predominantly found in nature. Enriching for deuterium, for example, may afford certain therapeutic advantages, such as increasing in vivo half-life or reducing dosage requirements, or may provide a compound useful as a standard for characterization of biological samples.

Isotopically-enriched Compound I described herein can be prepared without undue experimentation by conventional techniques well known to those skilled in the art or by processes analogous to those described in US 2014/0099283 A1 and US 2013/0315868 A1, the contents of which are hereby incorporated by reference in their entireties, using appropriate isotopically-enriched reagents and/or intermediates.

### Preparation of Compounds

Compound I or a pharmaceutically acceptable salt thereof can be prepared, according to the procedures described by US20130315868 and US20130315868A1, the contents of which are hereby incorporated by reference in their entireties. Different forms of Compound I or a pharmaceutically acceptable salt can prepared based on the guidance provided herein.

Samatasvir can be prepared according to the procedures described in U.S. Pat. No. 8,362,068 B2, the contents of which is hereby incorporated by reference in its entirety.

### Preparation of Solid Forms

In various embodiments, provided herein is a method of preparing Form I of Compound I. In some embodiments, the method comprises mixing a first solvent with Compound I to produce a first mixture. In some embodiments, the method comprises changing the temperature of the first mixture to a first temperature. In some embodiments, the method comprises adding a second solvent to the first mixture to produce a second mixture. In some embodiments, the method comprises changing the temperature of the second mixture to a second temperature. In some embodiments, the method comprises recovering solids from the first mixture or the second mixture. The first mixture or the second mixture each can be a solution, a suspension, or a mixture of a solution and solids.

The first solvent described herein can be n-heptane, diethyl ether, propyl acetate, ethyl acetate, isopropyl acetate (IPA), methyl isobutyl ketone (MIBK), 2-propanol, methyl ethyl ketone (MEK), 1-propanol, acetone, ethanol, dimethyl sulfoxide, water, tert-butylmethyl ether, 2-methyl-1-propanol, cyclohexane, 1,4-dioxane, toluene, chloroform, 1,2-dimethoxyethane, tetrahydrofuran (THF), dichloromethane, 2-methoxyethanol, methanol, N,N-dimethylformamide, acetonitrile, ethyleneglycol, nitromethane, N-methylpyrrolidone, or a mixture thereof. In an embodiment, the first solvent is n-heptane, diethyl ether, propyl acetate, ethyl acetate, isopropyl acetate (IPA), methyl isobutyl ketone (MIBK), 2-propanol, methyl ethyl ketone (MEK), 1-propanol, acetone, water, tert-butylmethyl ether, 2-methyl-1-propanol, cyclohexane, toluene, 1,2-dimethoxyethane, acetonitrile, nitromethane, or a mixture thereof. In an embodiment, the first solvent is n-heptane, diethyl ether, ethyl acetate, isopropyl acetate (IPA), 2-propanol, methyl ethyl ketone (MEK), water, tert-butylmethyl ether, 2-methyl-1-propanol, toluene, acetonitrile, nitromethane, or a mixture thereof. In an embodiment, the first solvent is propyl acetate, methyl isobutyl ketone (MIBK), 1-propanol, acetone, cyclohexane, 1,2-dimethoxyethane, or a mixture thereof. In an embodiment, the first solvent is propyl acetate or acetone.

The second solvent described herein can be n-heptane, diethyl ether, propyl acetate, ethyl acetate, isopropyl acetate (IPA), methyl isobutyl ketone (MIBK), 2-propanol, methyl ethyl ketone (MEK), 1-propanol, acetone, ethanol, dimethyl sulfoxide, water, tert-butylmethyl ether, 2-methyl-1-propanol, cyclohexane, 1,4-dioxane, toluene, chloroform, 1,2-dimethoxyethane, tetrahydrofuran (THF), dichloromethane, 2-methoxyethanol, methanol, N,N-dimethylformamide, acetonitrile, ethyleneglycol, nitromethane, N-methylpyrrolidone, or a mixture thereof. In an embodiment, the second solvent is n-heptane, diethyl ether, propyl acetate, ethyl acetate, isopropyl acetate (IPA), methyl isobutyl ketone (MIBK), 2-propanol, methyl ethyl ketone (MEK), 1-propanol, acetone, water, tert-butylmethyl ether, 2-methyl-1-propanol, cyclohexane, toluene, 1,2-dimethoxyethane, acetonitrile, nitromethane, or a mixture thereof. In an embodiment, the second solvent is n-heptane, diethyl ether, ethyl acetate, isopropyl acetate (IPA), 2-propanol, methyl ethyl ketone (MEK), water, tert-butylmethyl ether, 2-methyl-1-propanol, toluene, acetonitrile, nitromethane, or a mixture thereof. In an embodiment, the second solvent is propyl acetate, methyl isobutyl ketone (MIBK), 1-propanol, acetone, cyclohexane, 1,2-dimethoxyethane, or a mixture thereof. In an embodiment, the second solvent is propyl acetate or acetone. In an embodiment, the second solvent is heptanes, hexane, cyclohexaine, t-butyl methyl ether, diethyl ether, toluene, water, or a mixture thereof.

The first temperature described herein can be −5 to 100° C., optionally 0 to 90° C., optionally 0 to 10° C., optionally 65 to 85° C., optionally 70 to 80° C., optionally about 75° C. The second temperature described herein can be −5 to 65° C., optionally 0 to 60° C., optionally 0 to 10° C., optionally 40 to 60° C., optionally 40 to 50° C., optionally 50 to 60° C., optionally about 53° C., optionally 15 to 25° C., optionally about room temperature. In some embodiments, the method includes changing the temperature to a third temperature. The third temperature herein can be about −5° C. to about 30° C., optionally about 0 to about 25° C., optionally about 15° C. to about 25° C., optionally about 20° C. In some embodiments, the method includes maintaining a temperature for a certain period of time. It would be understood by a person with ordinary skill in the art that the sequence of the steps described above can change and any one step can be omitted or repeated, and that methods with varying sequence of the steps, omitted step(s), or repeating step(s) are within the scope of this invention.

As an example, the method can include (a) adding ethyl acetate to Compound I; (b) heating to a first temperature to produce a solution; (c) cooling the solution to a second temperature; (d) cooling the solution to a third temperature to produce a solid; and (e) recovering the solid.

As an example, the method can include (a) adding 2-propanol to Compound I; (b) heating to a first temperature to produce a solution; (c) cooling the solution to a second temperature; (d) adding n-heptane to the solution; (e) cooling the solution to a third temperature to produce a solid; and (f) recovering the solid.

In an embodiment, the solution in step b) is heated to about 70-80° C., optionally over about 50-70 minutes. In an embodiment, the solution in step b) is held at about 70-80° C. for about 35-50 minutes.

In an embodiment, the solution is filtered between step b) and step c). In an embodiment, additional 2-propanol is added to the solution before step c). In an embodiment, the solution is cooled to about 45-60° C. in step c), optionally over about 30-40 minutes.

In an embodiment, additional Compound I is added after step c).

In an embodiment, the solution is cooled in step e) to about 15-25° C., optionally over about 70-110 minutes. In an embodiment, the solution in step e) is held at 15-25° C. for about 11-15 hours.

In an embodiment, the solid produced in step e) is recovered by filtration. In an embodiment, the solid produced in step e) is recovered by filtration and washed with a mixture of 2-propanol and n-heptane. In an embodiment, the washed solid is deliquored, optionally for about 25-35 minutes. In an embodiment, the washed and/or deliquored solid is dried, optionally at about 55-65° C., optionally for about 19-23 hours, optionally under vacuum.

In various embodiments, the method provided herein comprises seeding. In various embodiments, the method provided herein does not include seeding.

In various embodiments, provided herein is Form I of Compound I produced by the method provided herein.

In various embodiments, provided herein is a method of preparing Form II of Compound I. In some embodiments, the method comprises maturing amorphous Compound I in acetone. In some embodiments, the method comprises slurrying Form I in cyclohexane. In some embodiments, the method comprises contacting Form I with the solvent 1,4-dioxane and the anti-solvent heptane.

In various embodiments, provided herein is Form II of Compound I produced by the method provided herein.

In various embodiments, provided herein is a method of preparing Form III of Compound I. In some embodiments, the method comprises maturing amorphous Compound I in propyl acetate. In some embodiments, the method comprises slurrying Form I in isopropyl acetate. In some embodiments, the method comprises slurrying Form I in isopropyl acetate.

In various embodiments, provided herein is Form III of Compound I produced by the method provided herein.

In various embodiments, provided herein is a method of preparing amorphous Compound I. In certain embodiments, Compound I is prepared by any technique apparent to those of skill in the art. In some embodiments, amorphous Compound I is prepared by lyophilization or freeze drying. In some embodiments, amorphous Compound I is prepared by freeze drying in a solvent. In particular embodiments, amorphous Compound I is prepared by freeze drying in t-BuOH and water, for instance 75/25 v/v t-BuOH/water. In some embodiments, amorphous Compound I is prepared by rapid evaporation of Compound I from a solution. In particular embodiments, the solution is in methanol.

In various embodiments, provided herein is amorphous Compound I produced by the method provided herein.

Additional steps and reagents not provided in this method would be known to those of skill in the art. Exemplary methods of preparation are described in detail in the examples below.

### Compositions and Methods of Administration

In certain embodiments, provided herein are compositions comprising one or more solid forms of Compound I. In some embodiments, the composition comprises one or more crystalline forms of Compound I. In certain embodiments, the composition comprises at least Form I of Compound I.

At least some of the compositions provided herein can include other components. In various embodiments, the composition includes Form I and a pharmaceutically acceptable carrier or excipient. In a specific embodiment and in this context, the term “pharmaceutically acceptable” means approved by a regulatory agency of the Federal or a state government or listed in the U.S. Pharmacopeia or other generally recognized pharmacopeia for use in animals, and more particularly in humans. Provided herein are also pharmaceutical compositions each including a solid form of Compound I. The solid form can include a crystalline form, for example, Form I, II, or III described herein.

In certain embodiments, the composition includes about 5%, about 10%, about 15%, about 20%, about 25%, about 30%, about 35%, about 40%, about 45%, about 50%, about 55%, about 60%, about 65%, about 70%, or about 75% by weight of Compound I, or an isotopic variant thereof, or a pharmaceutically acceptable salt thereof, or a pharmaceutically acceptable solvate thereof. In certain embodiments, the composition comprises about 4%, about 5%, about 6%, about 7%, about 8%, about 9%, about 10%, about 11%, about 12%, about 13%, or about 14% by weight of Compound I, or an isotopic variant thereof, or a pharmaceutically acceptable salt thereof, or a pharmaceutically acceptable solvate thereof. In certain embodiments, the composition comprises about 20%, about 21%, about 22%, about 23%, about 24%, about 25%, about 26%, about 27%, about 28%, about 29%, or about 30% by weight of Compound I, or an isotopic variant thereof, or a pharmaceutically acceptable salt thereof, or a pharmaceutically acceptable solvate thereof. In certain embodiments, the composition comprises about 45%, about 46%, about 48%, about 49%, about 50%, about 51%, about 52%, about 53%, about 54%, about 55%, or about 60% by weight of Compound I, or an isotopic variant thereof, or a pharmaceutically acceptable salt thereof, or a pharmaceutically acceptable solvate thereof. In certain embodiments, the composition comprises about 70%, about 71%, about 72%, about 73%, about 74%, about 75%, about 76%, about 77%, about 78%, about 79%, or about 80% by weight of Compound I, or an isotopic variant thereof, or a pharmaceutically acceptable salt thereof, or a pharmaceutically acceptable solvate thereof.

In some embodiments, at least some of Compound I is in a solid form, for example, a crystalline form. For example, at least some of Compound I in a composition provided herein is in Form I. In various embodiments, the composition includes about 5%, about 10%, about 15%, about 20%, about 25%, about 30%, about 35%, about 40%, about 45%, about 50%, about 55%, about 60%, about 65%, about 70%, or about 75% by weight of Form I of Compound I. In certain embodiments, the composition comprises about 4%, about 5%, about 6%, about 7%, about 8%, about 9%, about 10%, about 11%, about 12%, about 13%, or about 14% by weight of Form I of Compound I. In certain embodiments, the composition comprises about 20%, about 21%, about 22%, about 23%, about 24%, about 25%, about 26%, about 27%, about 28%, about 29%, or about 30% by weight of Form I of Compound I. In certain embodiments, the composition comprises about 45%, about 46%, about 48%, about 49%, about 50%, about 51%, about 52%, about 53%, about 54%, about 55%, or about 60% by weight of Form I of Compound I, or an isotopic variant thereof, or a pharmaceutically acceptable salt thereof, or a pharmaceutically acceptable solvate thereof.

In some embodiments, at least some of Compound I is in a solid form, for example, an amorphous form. For example, at least some of Compound I in a composition provided herein is in an amorphous form. In various embodiments, the composition includes about 5%, about 10%, about 15%, about 20%, about 25%, about 30%, about 35%, about 40%, about 45%, about 50%, about 55%, about 60%, about 65%, about 70%, or about 75% by weight of an amorphous form of Compound I. In certain embodiments, the composition comprises about 4%, about 5%, about 6%, about 7%, about 8%, about 9%, about 10%, about 11%, about 12%, about 13%, or about 14% by weight of an amorphous form of Compound I. In certain embodiments, the composition comprises about 20%, about 21%, about 22%, about 23%, about 24%, about 25%, about 26%, about 27%, about 28%, about 29%, or about 30% by weight of an amorphous form of Compound I. In certain embodiments, the composition comprises about 45%, about 46%, about 48%, about 49%, about 50%, about 51%, about 52%, about 53%, about 54%, about 55%, or about 60% by weight of an amorphous form of Compound I, or an isotopic variant thereof, or a pharmaceutically acceptable salt thereof, or a pharmaceutically acceptable solvate thereof.

In various embodiments, the composition provided herein comprises from about 5% to about 95%, about 5% to about 90%, about 5% to about 80%, about 10% to about 70%, about 15% to about 60%, about 20% to about 50%, from about 50% to about 95%, from about 50% to about 90%, from about 60% to about 90%, from about 60% to about 80%, or from about 70% to about 80% by weight of one or more excipients. In certain embodiments, the composition provided herein comprises about 95%, about 90%, about 85%, about 80%, about 75%, about 70%, about 65%, about 60%, about 55%, or about 50% by weight of one or more excipients. In certain embodiments, the composition provided herein comprises about 98%, about 97%, about 96%, about 95%, about 94%, about 93%, about 92%, about 91%, about 90%, about 89%, about 88%, about 87%, about 86%, or about 85% by weight of one or more excipients. In certain embodiments, the composition provided herein comprises about 85%, about 84%, about 83%, about 82%, about 80%, about 79%, about 78%, about 77%, about 76%, about 75%, about 74%, about 73%, about 72%, about 71%, about 70%, about 69%, about 68%, about 67%, about 66%, or about 65% by weight of one or more excipients. In certain embodiments, the composition provided herein comprises about 55%, about 54%, about 53%, about 52%, about 51%, about 50%, about 49%, about 48%, about 47%, about 46%, or about 45% by weight of one or more excipients. In certain embodiments, the composition provided herein comprises about 30%, about 29%, about 28%, about 27%, about 26%, about 25%, about 24%, about 23%, about 22%, about 21%, or about 20% by weight of one or more excipients.

Examples of excipients that can be used in the compositions provided herein include, but are not limited to, binders, fillers, disintegrants, and lubricants. Binders suitable for use in the pharmaceutical compositions provided herein include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre gelatinized starch, hydroxypropyl methyl cellulose, (e.g., Nos. 2208, 2906, 2910), microcrystalline cellulose, and mixtures thereof.

Examples of fillers suitable for use in the pharmaceutical compositions provided herein include, but are not limited to, talc, calcium carbonate (e.g., granules or powder), microcrystalline cellulose, powdered cellulose, dextrates, kaolin, mannitol, silicic acid, sorbitol, starch, pre gelatinized starch, and mixtures thereof. The binder or filler in pharmaceutical compositions is typically present in from about 50 to about 99 weight percent of the pharmaceutical composition or dosage form.

Suitable forms of microcrystalline cellulose include, but are not limited to, the materials sold as AVICEL PH 101, AVICEL PH 103 AVICEL RC 581, AVICEL PH 105 (available from FMC Corporation, American Viscose Division, Avicel Sales, Marcus Hook, Pa.), and mixtures thereof. A specific binder is a mixture of microcrystalline cellulose and sodium carboxymethyl cellulose sold as AVICEL RC 581. Suitable anhydrous or low moisture excipients or additives include AVICEL PH 103 and Starch 1500 LM.

Disintegrants can be used in the compositions to provide tablets that disintegrate when exposed to an aqueous environment. Tablets that contain too much disintegrant may disintegrate in storage, while those that contain too little may not disintegrate at a desired rate or under the desired conditions. Thus, a sufficient amount of disintegrant that is neither too much nor too little to detrimentally alter the release of the active ingredients should be used to form solid oral dosage forms. The amount of disintegrant used varies based upon the type of formulation, and is readily discernible to those of ordinary skill in the art. Typical pharmaceutical compositions comprise from about 0.5 to about 15 weight percent of disintegrant, specifically from about 1 to about 5 weight percent of disintegrant.

Disintegrants that can be used in the pharmaceutical compositions provided herein include, but are not limited to, agar, alginic acid, calcium carbonate, microcrystalline cellulose, croscarmellose sodium, crospovidone, polacrilin potassium, sodium starch glycolate, potato or tapioca starch, pre gelatinized starch, other starches, clays, other algins, other celluloses, gums, and mixtures thereof.

Lubricants that can be used in the pharmaceutical compositions provided herein include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl laureate, agar, and mixtures thereof. Additional lubricants include, for example, a syloid silica gel (AEROSIL 200, manufactured by W.R. Grace Co. of Baltimore, Md.), a coagulated aerosol of synthetic silica (marketed by Degussa Co. of Plano, Tex.), CAB O SIL (a pyrogenic silicon dioxide product sold by Cabot Co. of Boston, Mass.), and mixtures thereof. If used at all, lubricants are typically used in an amount of less than about 1 weight percent of the pharmaceutical compositions or dosage forms into which they are incorporated.

In an embodiment, the weight percentage of filler is about 15% to 60%, optionally about 20% to 60%, optionally about 25% to 55%, optionally about 30% to 50%, optionally about 35% to 60%, optionally about 50% to 60%.

In an embodiment, the weight percentage of disintegrant is about 1% to 25%, optionally about 2% to 20%, optionally about 5% to 15%, optionally about 8% to 12%, optionally about 10%.

In an embodiment, the weight percentage of lubricant is about 0.1% to about 3%, optionally about 0.5% to about 1%,

In an aspect, a pharmaceutical composition is provided which comprises 25 mg Compound I in a 100 mg tablet. In an aspect, a pharmaceutical composition is provided which comprises 50 mg Compound I in a 200 mg tablet. In an aspect, a pharmaceutical composition is provided which comprises 100 mg Compound I in a 400 mg tablet. In an aspect, a pharmaceutical composition is provided which comprises 150 mg Compound I in a 600 mg tablet. In an aspect, a pharmaceutical composition is provided which comprises 200 mg Compound I in a 800 mg tablet. In an aspect, a pharmaceutical composition is provided which comprises 200 mg Compound I in a 400 mg tablet. In an aspect, a pharmaceutical composition is provided which comprises 300 mg Compound I in a 600 mg tablet.

In an aspect, a pharmaceutical composition is provided which comprises 5 mg Compound I in a 15 mg mixture. In an aspect, a pharmaceutical composition is provided which comprises 10 mg Compound I in a 30 mg mixture. In an aspect, a pharmaceutical composition is provided which comprises 15 mg Compound I in a 45 mg mixture. In an aspect, a pharmaceutical composition is provided which comprises 20 mg Compound I in a 60 mg mixture. In an aspect, a pharmaceutical composition is provided which comprises 25 mg Compound I in a 75 mg mixture. In an aspect, a pharmaceutical composition is provided which comprises 30 mg Compound I in a 90 mg mixture. In an aspect, a pharmaceutical composition is provided which comprises 40 mg Compound I in a 120 mg mixture. In an aspect, a pharmaceutical composition is provided which comprises 50 mg Compound I in a 150 mg mixture. In various embodiments, the mixture referred herein is used to fill one or more capsules.

Other formulations having Compound I in spray-dried dispersions, melt extruded mixtures, nanoparticles, suspensions, solutions, pastes, gels, and exlixirs are also in the scope of the present invention. Other dosage forms, including other tablets (e.g., bilayer tablets with one layer comprising samatasvir in compositions provided herein and the other layer comprising Compound I, including Form I, in compositions provided herein); caplets; other capsules, such as soft elastic gelatin capsules; cachets; troches; lozenges; dispersions; suppositories; ointments; cataplasms (poultices); pastes; powders; dressings; creams; plasters; solutions; patches; aerosols (e.g., nasal sprays or inhalers); gels; liquid dosage forms suitable for oral or mucosal administration to a subject, including suspensions (e.g., aqueous or non-aqueous liquid suspensions, oil in water emulsions, or a water in oil liquid emulsions), solutions, and elixirs; liquid dosage forms suitable for parenteral administration to a subject; and sterile solids (e.g., crystalline or amorphous solids) that can be reconstituted to provide liquid dosage forms are also in the scope of the present invention.

In certain embodiments, the compounds and compositions provided herein comprise a second agent effective for the treatment of the disorder, such as HCV infection in a subject in need thereof. The second agent can be any agent known to those of skill in the art to be effective for the treatment of the disorder, including those currently approved by the FDA.

In certain embodiments, a compound provided herein is administered in combination with one second agent. In further embodiments, a compound provided herein is administered in combination with two second agents. In still further embodiments, a compound provided herein is administered in combination with two or more second agents.

As used herein, the term “in combination” includes the use of more than one therapy (e.g., one or more prophylactic and/or therapeutic agents). The use of the term “in combination” does not restrict the order in which therapies (e.g., prophylactic and/or therapeutic agents) are administered to a subject with a disorder. A first therapy (e.g., a prophylactic or therapeutic agent such as a compound provided herein) can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapy (e.g., a prophylactic or therapeutic agent) to a subject with a disorder.

As used herein, the term “synergistic” includes a combination of a compound provided herein and another therapy (e.g., a prophylactic or therapeutic agent) which has been or is currently being used to prevent, manage, or treat a disorder, which is more effective than the additive effects of the therapies. A synergistic effect of a combination of therapies (e.g., a combination of prophylactic or therapeutic agents) permits the use of lower dosages of one or more of the therapies and/or less frequent administration of said therapies to a subject with a disorder. The ability to utilize lower dosages of a therapy (e.g., a prophylactic or therapeutic agent) and/or to administer said therapy less frequently reduces the toxicity associated with the administration of said therapy to a subject without reducing the efficacy of said therapy in the prevention or treatment of a disorder). In addition, a synergistic effect can result in improved efficacy of agents in the prevention or treatment of a disorder. Finally, a synergistic effect of a combination of therapies (e.g., a combination of prophylactic or therapeutic agents) may avoid or reduce adverse or unwanted side effects associated with the use of either therapy alone.

The active compounds provided herein can be administered in combination or alternation with another therapeutic agent, in particular an anti-HCV agent. In combination therapy, effective dosages of two or more agents are administered together, whereas in alternation or sequential-step therapy, an effective dosage of each agent is administered serially or sequentially. The dosages given will depend on absorption, inactivation, and excretion rates of the drug as well as other factors known to those of skill in the art. It is to be noted that dosage values will also vary with the severity of the condition to be alleviated. It is to be further understood that for any particular subject, specific dosage regimens and schedules should be adjusted over time according to the individual need and the professional judgment of the person administering or supervising the administration of the compositions. In certain embodiments, an anti-HCV (or anti-pestivirus or anti-flavivirus) compound that exhibits an EC₅₀ of 10-15 μM is desirable. In certain embodiments, less than 1-5 M, is desirable.

It has been recognized that drug-resistant variants of flaviviruses, pestiviruses, or HCV can emerge after prolonged treatment with an antiviral agent. Drug resistance most typically occurs by mutation of a gene that encodes for an enzyme used in viral replication. The efficacy of a drug against the viral infection can be prolonged, augmented, or restored by administering the compound in combination or alternation with a second, and perhaps third, antiviral compound that induces a different mutation from that caused by the principle drug. Alternatively, the pharmacokinetics, biodistribution or other parameter of the drug can be altered by such combination or alternation therapy. In general, combination therapy is typically preferred over alternation therapy because it induces multiple simultaneous stresses on the virus.

In various embodiments, provided herein are compositions that include Compound I and a second therapeutic agent. For example, the second therapeutic agent can be a second anti-viral agent (including a second anti-HCV agent). In certain embodiments, the second anti-viral agent is a NS5A inhibitor. For example, the second anti-viral agent is samatasvir.

In certain embodiments provided herein are pharmaceutical compositions comprising intragranular components and extragranular components. In certain embodiments, the pharmaceutical compositions comprise Compound I in any form described herein including Form I, Form II, Form III, or an amorphous form. In particular embodiments, the pharmaceutical compositions comprise Form I. In further embodiments, the pharmaceutical compositions comprise samatasvir and Compound I together. In certain embodiments, the pharmaceutical compositions comprise samatasvir and Compound I in any form described herein including Form I, Form II, Form III, or an amorphous form. In particular embodiments, the pharmaceutical compositions comprise samatasvir and Form I. In particular embodiments, the samatasvir is Form A samatasvir or in spray-dried particles as described in U.S. provisional application No. 61/948,458, filed Mar. 5, 2014, entitled “SOLID FORMS OF A FLAVIVIRIDAE VIRUS INHIBITOR COMPOUND AND SALTS THEREOF,” bearing attorney docket no. 11874-295-888, and applicant docket no. IDX 1151, the contents of which are hereby incorporated by reference in their entirety.

In one embodiment, provided herein is a pharmaceutical composition comprising a compound provided herein, a dispersant, a disintegrant, a filler, a glidant, a lubricant, or a mixture thereof. In some embodiments, the compound in the pharmaceutical composition is selected from Compound I, samatasvir, an isotopic variant(s) thereof, or a pharmaceutically acceptable salt(s) thereof, or a pharmaceutically acceptable solvate(s) thereof, a solid form thereof, or a mixture thereof. In some embodiments, the pharmaceutical composition includes Compound I, samatasvir, and a pharmaceutically acceptable excipient selected from a disintegrant, a filler, a glidant, a lubricant, an organic acid, a surfactant, or a mixture thereof. In some embodiments, the pharmaceutical composition includes Form I of Compound I, samatasvir, and a pharmaceutically acceptable excipient selected from a disintegrant, a filler, a glidant, a lubricant, an organic acid, a surfactant, or a mixture thereof. In some embodiments, the pharmaceutical composition includes Form II of Compound I, samatasvir, and a pharmaceutically acceptable excipient selected from a disintegrant, a filler, a glidant, a lubricant, an organic acid, a surfactant, or a mixture thereof. In some embodiments, the pharmaceutical composition includes Form III of Compound I, samatasvir, and a pharmaceutically acceptable excipient selected from a disintegrant, a filler, a glidant, a lubricant, an organic acid, a surfactant, or a mixture thereof.

In one embodiment, provided herein is a pharmaceutical composition comprising a granular pharmaceutical composition. In one embodiment, the pharmaceutical composition includes a granular pharmaceutical composition and extragranular components. In some embodiments, the granular pharmaceutical composition includes spray-dried particles comprising a compound selected from samatasvir, its regioisomer, or a mixture thereof, or an isotopic variant thereof, or a pharmaceutically acceptable salt thereof, or a pharmaceutically acceptable solvate thereof; and a first pharmaceutically acceptable excipient. Various embodiments of the granular pharmaceutical composition and the spray-dried particles are provided in co-pending U.S. Patent Application Ser. No. 61/948,458, filed on Mar. 5, 2014, entitled “SOLID FORMS OF A FLAVIVIRIDAE VIRUS INHIBITOR COMPOUND AND SALTS THEREOF,” bearing attorney docket no. 11874-295-888, and applicant docket no. IDX 1151, the entirety of which is incorporated herein as if they are fully described herein.

In some embodiments, the granular pharmaceutical composition includes spray-dried particles comprising Compound I, or an isotopic variant thereof, or a pharmaceutically acceptable salt thereof, or a pharmaceutically acceptable solvate thereof; and a first pharmaceutically acceptable excipient.

In one embodiment, provided herein is a pharmaceutical composition comprising (i) one or more granular compositions each comprising intragranular components comprising a compound provided herein, a dispersant, a disintegrant, a filler, a glidant, a lubricant, or a mixture thereof; and optionally (ii) extragranular components comprising a compound provided herein, a disintegrant, a filler, a glidant, a lubricant, an organic acid, a surfactant, or a mixture thereof.

In another embodiment, provided herein is a pharmaceutical composition comprising (i) one or more granular compositions each comprising intragranular components comprising a compound provided herein, a dispersant, a glidant, a lubricant, or a mixture thereof; and optionally (ii) extragranular components comprising a compound provided herein, a disintegrant, a filler, a glidant, a lubricant, or a mixture thereof.

In yet another embodiment, provided herein is a pharmaceutical composition comprising (i) one or more granular compositions each comprising intragranular components comprising a compound provided herein, a dispersant, a glidant, a lubricant, or a mixture thereof; and optionally (ii) extragranular components comprising a compound provided herein, a disintegrant, a filler, a glidant, a lubricant, an organic acid, or a mixture thereof.

In yet another embodiment, provided herein is a pharmaceutical composition comprising (i) one or more granular compositions each comprising intragranular components comprising a compound provided herein, a dispersant, a glidant, a lubricant, or a mixture thereof; and optionally (ii) extragranular components comprising a compound provided herein, a disintegrant, a filler, a glidant, a lubricant, a surfactant, or a mixture thereof.

In yet another embodiment, provided herein is a pharmaceutical composition comprising (i) one or more granular compositions each comprising intragranular components comprising a compound provided herein, a dispersant, a glidant, a lubricant, or a mixture thereof; and optionally (ii) extragranular components comprising a compound provided herein, a disintegrant, a filler, a glidant, a lubricant, an organic acid, a surfactant, or a mixture thereof.

In still another embodiment, provided herein is a pharmaceutical composition comprising (i) one or more granular compositions each comprising intragranular components comprising a compound provided herein, a dispersant, a disintegrant, a filler, a glidant, a lubricant, or a mixture thereof; and optionally (ii) extragranular components comprising a compound provided herein, a glidant, a lubricant, a mixture thereof.

The compound provided herein as referred in various embodiments of the pharmaceutical composition provided herein can be selected from Compound I, samatasvir, an isotopic variant(s) thereof, a pharmaceutically acceptable salt(s) thereof, a pharmaceutically acceptable solvate(s) thereof, a solid form(s) thereof, or a mixture thereof.

In various embodiments, the intragranular components comprise samatasvir. In various embodiments, the intragranular components comprise Compound I. For example, Compound I can be in a crystalline form, for example, Form I, or an amorphous form. In some embodiments, the intragranular components comprise samatasvir and Compound I. In certain embodiments, the intragranular components comprise samatasvir and Form I of Compound I. For example, the intragranular components can be prepared by compacting spray-dried particles provided herein (having samatasvir, Form I of Compound I, or a mixture thereof) with a compound selected from samatasvir, Form I of Compound I, or a mixture thereof. In particular embodiments, the intragranular components are prepared by compacting spray-dried particles provided herein (having samatasvir) with Form I of Compound I. In certain embodiments, the intragranular components comprise samatasvir and an amorphous form of Compound I.

In various embodiments, the extragranular components comprise samatasvir. In various embodiments, the extragranular components comprise Compound I. For example, Compound I can be in a crystalline form, for example, Form I, or an amorphous form. In some embodiments, the extragranular components comprise samatasvir and Compound I. In certain embodiments, the extragranular components comprise samatasvir and Form I of Compound I. In certain embodiments, the extragranular components comprise samatasvir and an amorphous form of Compound I.

In various embodiments, the pharmaceutical composition provided herein comprises from about 5 to about 50% by weight of the granular composition, the components of which are described in further details herein. In some embodiments, the pharmaceutical composition provided herein comprises from about 10 to about 40% by weight of the granular composition, the components of which are described in further details herein. In some embodiments, the pharmaceutical composition provided herein comprises from about 15 to about 30% by weight of the granular composition, the components of which are described in further details herein. In certain embodiments, the pharmaceutical composition provided herein comprises about 20%, about 21%, about 22%, about 23%, about 24%, about 25%, about 26%, about 27%, about 28%, about 29%, or about 30% by weight of the granular composition, the components of which are described in further details herein.

In certain embodiments, the pharmaceutical composition provided herein comprises from about 5 to about 50%, from about 10 to about 50%, from about 15 to about 30%, or from about 15 to about 25% by weight of an intragranular dispersant. In certain embodiments, the pharmaceutical composition provided herein comprises about 15%, about 16%, about 17%, about 18%, about 19%, about 20%, about 21%, about 22%, about 23%, about 24%, about 25%, about 26%, about 27%, about 28%, about 29%, or about 30% by weight of an intragranular dispersant. In certain embodiments, the pharmaceutical composition provided herein comprises about 19% by weight of an intragranular dispersant. In certain embodiments, the pharmaceutical composition provided herein comprises about 25% by weight of an intragranular dispersant.

In certain embodiments, the intragranular dispersant is a hypromellose, a hypromellose acetate succinate, a hypromellose phthalate, a methacrylic acid and ethyl acrylate copolymer, a poloxamer, a polyethylene glycol, a povidone, a tocopherol polyethylene glycol succinate, or a mixture thereof. In certain embodiments, the intragranular dispersant is a povidone. In certain embodiments, the intragranular dispersant is a povidone having an average molecular weight from about 30,000 Da to about 70,000 Da or from about 40,000 Da to about 60,000 Da. In certain embodiments, the intragranular dispersant is a povidone having an average molecular weight of about 40,000 Da, 45,000 Da, 50,000 Da, or 55,000 Da. In certain embodiments, the intragranular dispersant is PVP-K30. In certain embodiments, the intragranular dispersant is a hypromellose acetate succinate. In certain embodiments, the intragranular dispersant is a hypromellose acetate succinate having an average molecular weight of about 18,000 Da. In certain embodiments, the intragranular dispersant is HPMCAS, MF grade. In certain embodiments, one intragranular dispersant is preferred over another. In some embodiments, povidone stabilizes a pharmaceutical composition described herein. Without being limited to any particular theory, povidone may provide certain interaction (for example, hydrogen bonding, dipole-dipole interaction, van del Waals forces, or other interactions) between the dispersant molecule and a compound provided herein (e.g., samatasvir, its isomer, or Compound I), which can stabilize the composition, for example, through colloidal formation or other stabilization mechanism.

In certain embodiments, the pharmaceutical composition provided herein comprises from about 1 to about 50%, from about 2 to about 20%, or from about 5 to about 15% by weight of an intragranular disintegrant. In certain embodiments, the pharmaceutical composition provided herein comprises about 5%, about 6%, about 7%, about 8%, about 9%, about 10%, about 11%, about 12%, about 13%, about 14%, or about 15% by weight of an intragranular disintegrant. In certain embodiments, the pharmaceutical composition provided herein comprises about 6% by weight of an intragranular disintegrant. In certain embodiments, the pharmaceutical composition provided herein comprises about 10% by weight of an intragranular disintegrant. In certain embodiments, the intragranular disintegrant is a crosslinked polyvinyl pyrrolidone or croscarmellose sodium. In certain embodiments, the intragranular disintegrant is a crosslinked polyvinyl pyrrolidone. In certain embodiments, the intragranular disintegrant is POLYPLASDONE® XL. In certain embodiments, the intragranular disintegrant is croscarmellose sodium. In certain embodiments, the intragranular disintegrant is AC-DI-SOL®.

In certain embodiments, the pharmaceutical composition provided herein comprises from about 1 to about 50%, from about 2 to about 20%, or from about 5 to about 15% by weight of an extragranular disintegrant. In certain embodiments, the pharmaceutical composition provided herein comprises about 5%, about 6%, about 7%, about 8%, about 9%, about 10%, about 11%, about 12%, about 13%, about 14%, or about 15% by weight of an extragranular disintegrant. In certain embodiments, the pharmaceutical composition provided herein comprises about 6% by weight of an extragranular disintegrant. In certain embodiments, the pharmaceutical composition provided herein comprises about 10% by weight of an extragranular disintegrant. In certain embodiments, the extragranular disintegrant is a crosslinked polyvinyl pyrrolidone or croscarmellose sodium. In certain embodiments, the extragranular disintegrant is a crosslinked polyvinyl pyrrolidone. In certain embodiments, the extragranular disintegrant is POLYPLASDONE® XL. In certain embodiments, the extragranular disintegrant is croscarmellose sodium. In certain embodiments, the extragranular disintegrant is AC-DI-SOL®.

In certain embodiments, the pharmaceutical composition provided herein comprises from about 10 to about 95%, from about 20 to about 90%, from about 25 to about 75%, or from about 30 to about 70% by weight of an intragranular filler. In certain embodiments, the pharmaceutical composition provided herein comprises about 10%, about 15%, about 20%, about 25%, about 30%, about 35%, about 40%, about 45%, about 50%, about 55%, about 60%, about 65%, about 70%, or about 75% by weight of an intragranular filler. In certain embodiments, the pharmaceutical composition provided herein comprises about 30%, about 50%, or about 65% by weight of an intragranular filler. In certain embodiments, the intragranular filler is a microcrystalline cellulose, lactose, or a mixture thereof. In certain embodiments, the intragranular filler is a microcrystalline cellulose. In certain embodiments, the intragranular filler is AVICEL® PH 102. In certain embodiments, the intragranular filler is lactose. In certain embodiments, the intragranular filler is lactose FAST FLO® 316. In certain embodiments, the intragranular filler is a mixture of microcrystalline cellulose and lactose. In certain embodiments, the intragranular filler is a mixture of AVICEL® PH 102 and lactose FAST FLO® 316.

In certain embodiments, the pharmaceutical composition provided herein comprises from about 10 to about 95%, from about 20 to about 90%, from about 25 to about 75%, or from about 30 to about 70% by weight of an extragranular filler. In certain embodiments, the pharmaceutical composition provided herein comprises about 25%, about 30%, about 35%, about 40%, about 45%, about 50%, about 55%, about 60%, about 65%, about 70%, or about 75% by weight of an extragranular filler. In certain embodiments, the pharmaceutical composition provided herein comprises about 30%, about 50%, or about 65% by weight of an extragranular filler. In certain embodiments, the extragranular filler is a microcrystalline cellulose, lactose, or a mixture thereof. In certain embodiments, the extragranular filler is a microcrystalline cellulose. In certain embodiments, the extragranular filler is AVICEL® PH 102. In certain embodiments, the extragranular filler is lactose. In certain embodiments, the extragranular filler is lactose FAST FLO® 316. In certain embodiments, the extragranular filler is a mixture of microcrystalline cellulose and lactose. In certain embodiments, the extragranular filler is a mixture of AVICEL® PH 102 and lactose FAST FLO® 316.

In certain embodiments, the pharmaceutical composition provided herein comprises from about 0.1 to about 5%, from about 0.1 to about 2%, from about 0.2 to about 1.5%, or from about 0.2 to about 1% by weight of an intragranular glidant. In certain embodiments, the pharmaceutical composition provided herein comprises about 0.1%, about 0.2%, about 0.3%, about 0.4%, about 0.5%, about 0.6%, about 0.7%, about 0.8%, about 0.9%, about 1%, about 1.1%, about 1.2%, about 1.3%, about 1.4%, or about 1.5% by weight of an intragranular glidant. In certain embodiments, the pharmaceutical composition provided herein comprises from about 0.1 to about 0.5% by weight of an intragranular glidant. In certain embodiments, the intragranular glidant is a colloidal silicon dioxide. In certain embodiment, the intragranular glidant is CAB-O-SIL®.

In certain embodiments, the pharmaceutical composition provided herein comprises from about 0.1 to about 5%, from about 0.1 to about 2%, from about 0.2 to about 1.5%, or from about 0.2 to about 1% by weight of an extragranular glidant. In certain embodiments, the pharmaceutical composition provided herein comprises about 0.1%, about 0.2%, about 0.3%, about 0.4%, about 0.5%, about 0.6%, about 0.7%, about 0.8%, about 0.9%, about 1%, about 1.1%, about 1.2%, about 1.3%, about 1.4%, or about 1.5% by weight of an extragranular glidant. In certain embodiments, the pharmaceutical composition provided herein comprises from about 0.1 to about 0.5% by weight of an extragranular glidant. In certain embodiments, the extragranular glidant is a colloidal silicon dioxide. In certain embodiment, the extragranular glidant is CAB-O-SIL®.

In certain embodiments, the pharmaceutical composition provided herein comprises from about 0.1 to about 3%, about 0.1 to about 2%, from about 0.1 to about 1%, or about 0.5 to about 1% by weight of an intragranular lubricant. In certain embodiments, the pharmaceutical composition provided herein comprises about 0.1%, about 0.2%, about 0.3%, by weight of an intragranular lubricant. In certain embodiments, the pharmaceutical composition provided herein comprises from about 0.1 to about 0.25% or about 0.5 to about 1% by weight of an intragranular lubricant. In certain embodiments, the intragranular lubricant is magnesium stearate.

In certain embodiments, the pharmaceutical composition provided herein comprises from about 0.1 to about 3%, 0.1 to about 2%, from about 0.1 to about 1%, from about 0.2 to about 0.5%, or about 0.5 to about 1% by weight of an extragranular lubricant. In certain embodiments, the pharmaceutical composition provided herein comprises about 0.1%, about 0.2%, about 0.3%, about 0.4%, about 0.5%, about 0.6%, about 0.7%, about 0.8%, about 0.9%, or about 1% by weight of an extragranular lubricant. In certain embodiments, the pharmaceutical composition provided herein comprises from about 0.25 to about 0.5% or about 0.5 to about 1% by weight of an extragranular lubricant. In certain embodiments, the extragranular lubricant is magnesium stearate.

In certain embodiments, the pharmaceutical composition provided herein comprises from about 1 to about 25%, from about 2 to about 20%, from about 5 to about 15%, or from about 10 to about 15% by weight of an extragranular organic acid. In certain embodiments, the pharmaceutical composition provided herein comprises about 5%, about 6%, about 7%, about 8%, about 9%, about 10%, about 11%, about 12%, about 13%, about 14%, or about 15% by weight of an extragranular organic acid. In certain embodiments, the pharmaceutical composition provided herein comprises from about 10 to about 15% by weight of an extragranular organic acid. In certain embodiments, the extragranular organic acid is tartaric acid.

In certain embodiments, the pharmaceutical composition provided herein comprises from about 1 to about 30%, from about 2 to about 25%, or from about 5 to about 25% by weight of an extragranular surfactant. In certain embodiments, the pharmaceutical composition provided herein comprises about 10%, about 11%, about 12%, about 13%, about 14%, about 15%, about 16%, about 17%, about 18%, about 19%, or about 20% by weight of an extragranular surfactant. In certain embodiments, the pharmaceutical composition provided herein comprises from about 10 to about 20% by weight of an extragranular surfactant. In certain embodiments, the extragranular surfactant is sodium lauryl sulfate.

In one embodiment, provided herein is a pharmaceutical composition comprising (i) intragranular components comprising: from about 5 to about 20% by weight of compound provided herein (e.g., Compound I, or samatasvir and Compound I), or an isotopic variant(s) thereof, or a pharmaceutically acceptable salt(s) thereof, or a pharmaceutically acceptable solvate(s) thereof; from about 10 to about 25% by weight of a dispersant, from about 0.1 to about 5% by weight of a glidant, and from about 0.05 to about 3% by weight of a lubricant; and optionally (ii) extragranular components comprising: from about 5 to about 20% by weight of compound provided herein (e.g., Compound I, or samatasvir and Compound I), or an isotopic variant(s) thereof, or a pharmaceutically acceptable salt(s) thereof, or a pharmaceutically acceptable solvate(s) thereof; from about 5 to about 25% by weight of a disintegrant, from about 25 to about 80% by weight of a filler, from about 0.1 to about 5% by weight of a glidant, and from about 0.05 to about 3% by weight of a lubricant.

In another embodiment, provided herein is a pharmaceutical composition comprising (i) intragranular components comprising: from about 5 to about 10% by weight of compound provided herein (e.g., Compound I, or samatasvir and Compound I), or an isotopic variant(s) thereof, or a pharmaceutically acceptable salt(s) thereof, or a pharmaceutically acceptable solvate(s) thereof; from about 15 to about 25% by weight of a dispersant, from about 0.2 to about 1.5% by weight of a glidant, and from about 0.05 to about 0.5% (or about 0.5% to about 1%) by weight of a lubricant; and optionally (ii) extragranular components comprising: from about 5 to about 20% by weight of compound provided herein (e.g., Compound I, or samatasvir and Compound I), or an isotopic variant(s) thereof, or a pharmaceutically acceptable salt(s) thereof, or a pharmaceutically acceptable solvate(s) thereof; from about 5 to about 15% by weight of a disintegrant, from about 30 to about 70% by weight of a filler, from about 0.2 to about 2% by weight of a glidant, and from about 0.1 to about 1% by weight of a lubricant.

In yet another embodiment, provided herein is a pharmaceutical composition comprising (i) intragranular components comprising: from about 6 to about 9% by weight of compound provided herein (e.g., Compound I, or samatasvir and Compound I), or an isotopic variant(s) thereof, or a pharmaceutically acceptable salt(s) thereof, or a pharmaceutically acceptable solvate(s) thereof; from about 18 to about 25% by weight of a dispersant, from about 0.5 to about 1.5% by weight of a glidant, and from about 0.05 to about 0.3% (or about 0.5% to about 1%) by weight of a lubricant; and optionally (ii) extragranular components comprising: from about 5 to about 20% by weight of compound provided herein (e.g., Compound I, or samatasvir and Compound I), or an isotopic variant(s) thereof, or a pharmaceutically acceptable salt(s) thereof, or a pharmaceutically acceptable solvate(s) thereof; from about 6 to about 10% by weight of a disintegrant, from about 30 to about 70% by weight of a filler, from about 0.2 to about 1% by weight of a glidant, and from about 0.1 to about 0.5% (or about 0.5% to about 1%) by weight of a lubricant.

In still another embodiment, provided herein is a pharmaceutical composition comprising (i) intragranular components comprising: from about 6 to about 9% by weight of compound provided herein (e.g., Compound I, or samatasvir and Compound I), or an isotopic variant(s) thereof, or a pharmaceutically acceptable salt(s) thereof, or a pharmaceutically acceptable solvate(s) thereof; from about 18 to about 25% by weight of a dispersant, from about 0.5 to about 1.5% by weight of a glidant, and from about 0.05 to about 0.3% (or about 0.5% to about 1%) by weight of a lubricant; and optionally (ii) extragranular components comprising: from about 5 to about 20% by weight of compound provided herein (e.g., Compound I, or samatasvir and Compound I), or an isotopic variant(s) thereof, or a pharmaceutically acceptable salt(s) thereof, or a pharmaceutically acceptable solvate(s) thereof; about 10% by weight of a disintegrant, about 65% by weight of a filler, about 0.5% by weight of a glidant, and from about 0.2 to about 0.5% (or about 0.5% to about 1%) by weight of a lubricant.

In one embodiment, provided herein is a pharmaceutical composition comprising (i) intragranular components comprising: from about 5 to about 20% by weight of samatasvir; from about 10 to about 25% by weight of a polyvinyl pyrrolidone (e.g., PVP-K30), from about 0.1 to about 5% by weight of a colloidal silicon dioxide (e.g., CAB-O-SIL® M-5P), and from about 0.05 to about 2% by weight of magnesium stearate; and optionally (ii) extragranular components comprising: from about 5 to about 20% by weight Compound I; from about 5 to about 25% by weight of a polyvinyl pyrrolidone (e.g., POLYPLASDONE® XL), from about 25 to about 80% by weight of a microcrystalline cellulose (e.g., AVICEL® PH 102), from about 0.1 to about 5% by weight of a glidant, and from about 0.05 to about 2% by weight of magnesium stearate.

In another embodiment, provided herein is a pharmaceutical composition comprising (i) intragranular components comprising: from about 5 to about 10% by weight of samatasvir; from about 15 to about 25% by weight of a polyvinyl pyrrolidone (e.g., PVP-K30), from about 0.2 to about 1.5% by weight of a colloidal silicon dioxide (e.g., CAB-O-SIL® M-5P), and from about 0.05 to about 0.5% by weight of magnesium stearate; and optionally (ii) extragranular components comprising: from about 5 to about 20% by weight Compound I; from about 5 to about 15% by weight of a polyvinyl pyrrolidone (e.g., POLYPLASDONE® Xl), from about 30 to about 70% by weight of a microcrystalline cellulose (e.g., AVICEL® PH 102), from about 0.2 to about 2% by weight of a glidant, and from about 0.1 to about 1% by weight of magnesium stearate.

In yet another embodiment, provided herein is a pharmaceutical composition comprising (i) intragranular components comprising: from about 6 to about 9% by weight of samatasvir; from about 18 to about 25% by weight of a polyvinyl pyrrolidone (e.g., PVP-K30), from about 0.5 to about 1.5% by weight of a colloidal silicon dioxide (e.g., CAB-O-SIL® M-5P), and from about 0.05 to about 0.3% by weight of magnesium stearate; and optionally (ii) extragranular components comprising: from about 5 to about 20% by weight Compound I; from about 6 to about 10% by weight of a polyvinyl pyrrolidone (e.g., POLYPLASDONE® XL), from about 30 to about 70% by weight of a microcrystalline cellulose (e.g., AVICEL® PH 102), from about 0.2 to about 1% by weight of a colloidal silicon dioxide (e.g., CAB-O-SIL® M-5P), and from about 0.1 to about 0.5% by weight magnesium stearate.

In still another embodiment, provided herein is a pharmaceutical composition comprising (i) intragranular components comprising: from about 6 to about 9% by weight of samatasvir; from about 18 to about 25% by weight of a polyvinyl pyrrolidone (e.g., PVP-K30), from about 0.5 to about 1.5% by weight of a colloidal silicon dioxide (e.g., CAB-O-SIL® M-5P), and from about 0.05 to about 0.3% by weight of magnesium stearate; and optionally (ii) extragranular components comprising: from about 5 to about 20% by weight Compound I; about 10% by weight of a polyvinyl pyrrolidone (e.g., POLYPLASDONE® XL), about 65% by weight of a microcrystalline cellulose (e.g., AVICEL® PH 102), about 0.5% by weight of a colloidal silicon dioxide (e.g., CAB-O-SIL® M-5P), and from about 0.2 to about 0.5% by weight of magnesium stearate.

In one embodiment, provided herein is a pharmaceutical composition comprising (i) intragranular components comprising: from about 5 to about 20% by weight of samatasvir; from about 5 to about 20% by weight Compound I; from about 10 to about 25% by weight of a polyvinyl pyrrolidone (e.g., PVP-K30), from about 0.1 to about 5% by weight of a colloidal silicon dioxide (e.g., CAB-O-SIL® M-5P), and from about 0.05 to about 2% by weight of magnesium stearate; and (ii) extragranular components comprising: from about 5 to about 25% by weight of a polyvinyl pyrrolidone (e.g., POLYPLASDONE® XL), from about 25 to about 80% by weight of a microcrystalline cellulose (e.g., AVICEL® PH 102), from about 0.1 to about 5% by weight of a glidant, and from about 0.05 to about 2% by weight of magnesium stearate.

In another embodiment, provided herein is a pharmaceutical composition comprising (i) intragranular components comprising: from about 5 to about 10% by weight of samatasvir; from about 5 to about 20% by weight Compound I; from about 15 to about 25% by weight of a polyvinyl pyrrolidone (e.g., PVP-K30), from about 0.2 to about 1.5% by weight of a colloidal silicon dioxide (e.g., CAB-O-SIL® M-5P), and from about 0.05 to about 0.5% by weight of magnesium stearate; and (ii) extragranular components comprising: from about 5 to about 15% by weight of a polyvinyl pyrrolidone (e.g., POLYPLASDONE® Xl), from about 30 to about 70% by weight of a microcrystalline cellulose (e.g., AVICEL® PH 102), from about 0.2 to about 2% by weight of a glidant, and from about 0.1 to about 1% by weight of magnesium stearate.

In yet another embodiment, provided herein is a pharmaceutical composition comprising (i) intragranular components comprising: from about 6 to about 9% by weight of samatasvir; from about 5 to about 20% by weight Compound I; from about 18 to about 25% by weight of a polyvinyl pyrrolidone (e.g., PVP-K30), from about 0.5 to about 1.5% by weight of a colloidal silicon dioxide (e.g., CAB-O-SIL® M-5P), and from about 0.05 to about 0.3% by weight of magnesium stearate; and (ii) extragranular components comprising: from about 6 to about 10% by weight of a polyvinyl pyrrolidone (e.g., POLYPLASDONE® XL), from about 30 to about 70% by weight of a microcrystalline cellulose (e.g., AVICEL® PH 102), from about 0.2 to about 1% by weight of a colloidal silicon dioxide (e.g., CAB-O-SIL® M-5P), and from about 0.1 to about 0.5% by weight magnesium stearate.

In still another embodiment, provided herein is a pharmaceutical composition comprising (i) intragranular components comprising: from about 6 to about 9% by weight of samatasvir; from about 5 to about 20% by weight Compound I; from about 18 to about 25% by weight of a polyvinyl pyrrolidone (e.g., PVP-K30), from about 0.5 to about 1.5% by weight of a colloidal silicon dioxide (e.g., CAB-O-SIL® M-5P), and from about 0.05 to about 0.3% by weight of magnesium stearate; and (ii) extragranular components comprising: about 10% by weight of a polyvinyl pyrrolidone (e.g., POLYPLASDONE® XL), about 65% by weight of a microcrystalline cellulose (e.g., AVICEL® PH 102), about 0.5% by weight of a colloidal silicon dioxide (e.g., CAB-O-SIL® M-5P), and from about 0.2 to about 0.5% by weight of magnesium stearate.

In one embodiment, provided herein is a pharmaceutical composition comprising (i) intragranular components comprising: spray-dried particles provided herein and a pharmaceutically acceptable excipient; and (ii) extragranular components comprising a pharmaceutically acceptable excipient.

In one embodiment, provided herein is a pharmaceutical composition comprising (i) intragranular components comprising: spray-dried particles provided herein and a pharmaceutically acceptable excipient; and (ii) extragranular components comprising: a compound provided herein (e.g., Compound I, or samatasvir and Compound I); and a pharmaceutically acceptable excipient.

In certain embodiments, the pharmaceutical composition provided herein comprises from about 10 to about 90%, from about 10 to about 50%, from about 20 to about 50%, or from about 20 to about 40% by weight of spray-dried particles as an intragranular component. In certain embodiments, the pharmaceutical composition provided herein comprises about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, or about 40% by weight of spray dried particles as an intragranular component. In certain embodiments, the pharmaceutical composition provided herein comprises from about 20 to about 40% by weight of spray dried particles as an intragranular component.

In certain embodiments, the pharmaceutical composition provided herein comprises from about 0.1 to about 5%, from about 0.1 to about 2%, from about 0.2 to about 1.5%, or from about 0.5 to about 1% by weight of an intragranular excipient. In certain embodiments, the pharmaceutical composition provided herein comprises about 0.1%, about 0.2%, about 0.3%, about 0.4%, about 0.5%, about 0.6%, about 0.7%, about 0.8%, about 0.9%, about 1%, about 1.1%, about 1.2%, about 1.3%, about 1.4%, or about 1.5% by weight of an intragranular excipient.

In certain embodiments, the pharmaceutical composition provided herein comprises from about 10 to about 90%, from about 50 to about 90%, from about 50 to about 80%, or from about 60 to about 75% by weight of an intragranular excipient. In certain embodiments, the pharmaceutical composition provided herein comprises about 60%, about 62%, about 64%, about 65%, about 66%, about 68%, about 70%, about 72%, about 74%, about 75%, about 76%, about 78%, or about 80% by weight of an intragranular excipient. In certain embodiments, the pharmaceutical composition provided herein comprises about 65% by weight of an intragranular excipient. In certain embodiments, the pharmaceutical composition provided herein comprises about 75% by weight of an intragranular excipient.

In certain embodiments, the pharmaceutical composition provided herein comprises from about 0.1 to about 5%, from about 0.1 to about 2%, from about 0.2 to about 1.5%, or from about 0.5 to about 1% by weight of an extragranular excipient. In certain embodiments, the pharmaceutical composition provided herein comprises about 0.1%, about 0.2%, about 0.3%, about 0.4%, about 0.5%, about 0.6%, about 0.7%, about 0.8%, about 0.9%, about 1%, about 1.1%, about 1.2%, about 1.3%, about 1.4%, or about 1.5% by weight of an extragranular excipient.

In certain embodiments, the pharmaceutical composition provided herein comprises from about 10 to about 90%, from about 50 to about 90%, from about 50 to about 80%, or from about 60 to about 75% by weight of an extragranular excipient. In certain embodiments, the pharmaceutical composition provided herein comprises about 60%, about 62%, about 64%, about 65%, about 66%, about 68%, about 70%, about 72%, about 74%, about 75%, about 76%, about 78%, or about 80% by weight of an extragranular excipient. In certain embodiments, the pharmaceutical composition provided herein comprises about 65% by weight of an extragranular excipient. In certain embodiments, the pharmaceutical composition provided herein comprises about 75% by weight of an extragranular excipient.

In one embodiment, provided herein is a pharmaceutical composition comprising: (i) intragranular components comprising: from about 10 to about 50% by weight of spray-dried particles provided herein and from about 0.1 to about 10% by weight of a pharmaceutically acceptable excipient, wherein the spray-dried particles optionally comprise a compound provided herein (e.g., Compound I, samatasvir, or a mixture thereof); and (ii) extragranular components comprising: from about 5% to about 20% of a compound provided herein (e.g., Compound I, samatasvir, or a mixture thereof) and from about 40 to about 90% by weight of a pharmaceutically acceptable excipient.

In one embodiment, provided herein is a pharmaceutical composition comprising: (i) intragranular components comprising: from about 10 to about 50% by weight of spray-dried particles provided herein and from about 0.1 to about 10% by weight of a pharmaceutically acceptable excipient, wherein the spray-dried particles comprise a compound provided herein (e.g., Compound I, samatasvir, or a mixture thereof); and (ii) extragranular components comprising: from about 40 to about 90% by weight of a pharmaceutically acceptable excipient and optionally from about 5% to about 20% of a compound provided herein (e.g., Compound I, samatasvir, or a mixture thereof).

In one embodiment, provided herein is a pharmaceutical composition comprising: (i) intragranular components comprising: from about 20 to about 50% by weight of spray-dried particles provided herein and from about 0.1 to about 5% by weight of a pharmaceutically acceptable excipient, wherein the spray-dried particles optionally comprise a compound provided herein (e.g., Compound I, samatasvir, or a mixture thereof); and (ii) extragranular components comprising: from about 5% to about 15% of a compound provided herein (e.g., Compound I, samatasvir, or a mixture thereof) and from about 65 to about 80% by weight of a pharmaceutically acceptable excipient.

In one embodiment, provided herein is a pharmaceutical composition comprising: (i) intragranular components comprising: from about 20 to about 50% by weight of spray-dried particles provided herein and from about 0.1 to about 5% by weight of a pharmaceutically acceptable excipient, wherein the spray-dried particles comprise a compound provided herein (e.g., Compound I, samatasvir, or a mixture thereof); and (ii) extragranular components comprising: from about 65 to about 80% by weight of a pharmaceutically acceptable excipient and optionally from about 5% to about 15% of a compound provided herein (e.g., Compound I, samatasvir, or a mixture thereof).

In one embodiment, provided herein is a pharmaceutical composition comprising: (i) intragranular components comprising: from about 25 to about 35% by weight of spray-dried particles provided herein and from about 0.5 to about 2% by weight of a pharmaceutically acceptable excipient, wherein the spray-dried particles optionally comprise a compound provided herein (e.g., Compound I, samatasvir, or a mixture thereof); and (ii) extragranular components comprising: from about 5% to about 9.5% of a compound provided herein (e.g., Compound I, samatasvir, or a mixture thereof) and from about 65 to about 75% by weight of a pharmaceutically acceptable excipient.

In one embodiment, provided herein is a pharmaceutical composition comprising: (i) intragranular components comprising: from about 25 to about 35% by weight of spray-dried particles provided herein and from about 0.5 to about 2% by weight of a pharmaceutically acceptable excipient, wherein the spray-dried particles comprise a compound provided herein (e.g., Compound I, samatasvir, or a mixture thereof); and (ii) extragranular components comprising: from about 65 to about 75% by weight of a pharmaceutically acceptable excipient and optionally from about 5% to about 9.5% of a compound provided herein (e.g., Compound I, samatasvir, or a mixture thereof).

In certain embodiments, the intragranular excipient is a disintegrant, a filler, a glidant, a lubricant, an organic acid, a surfactant, or a mixture thereof. In certain embodiments, the extragranular excipient is a disintegrant, a filler, a glidant, a lubricant, an organic acid, a surfactant, or a mixture thereof.

In one embodiment, provided herein is a pharmaceutical composition comprising (i) intragranular components comprising: spray-dried particles provided herein, and a disintegrant, a filler, a glidant, a lubricant, or a mixture thereof; and (ii) extragranular components comprising: a disintegrant, a filler, a glidant, a lubricant, an organic acid, a surfactant, or a mixture thereof, and (iii) a compound provided herein (e.g., Compound I, samatasvir, or a mixture thereof) in the spray-dried particles, in extragranular components, or in both. In certain embodiments, the compound is in the spray-dried particles. In certain embodiments, the compound is in the extragranular components. In certain embodiments, the compound is divided between the spray-dried particles and the extragranular components.

In another embodiment, provided herein is a pharmaceutical composition comprising (i) intragranular components comprising: spray-dried particles provided herein, a glidant, and a lubricant; and (ii) extragranular components comprising: a disintegrant, a filler, a glidant, and a lubricant, and (iii) a compound provided herein (e.g., Compound I, samatasvir, or a mixture thereof) in the spray-dried particles, in extragranular components, or in both. In certain embodiments, the compound is in the spray-dried particles. In certain embodiments, the compound is in the extragranular components. In certain embodiments, the compound is divided between the spray-dried particles and the extragranular components.

In yet another embodiment, provided herein is a pharmaceutical composition comprising (i) intragranular components comprising: spray-dried particles provided herein; and a glidant and a lubricant; and (ii) extragranular components comprising: a disintegrant, a filler, a glidant, a lubricant, and an organic acid, and (iii) a compound provided herein (e.g., Compound I, samatasvir, or a mixture thereof) in the spray-dried particles, in extragranular components, or in both. In certain embodiments, the compound is in the spray-dried particles. In certain embodiments, the compound is in the extragranular components. In certain embodiments, the compound is divided between the spray-dried particles and the extragranular components.

In yet another embodiment, provided herein is a pharmaceutical composition comprising (i) intragranular components comprising: spray-dried particles provided herein, a glidant, and a lubricant; and (ii) extragranular components comprising: a disintegrant, a filler, a glidant, a lubricant, and a surfactant, and (iii) a compound provided herein (e.g., Compound I, samatasvir, or a mixture thereof) in the spray-dried particles, in extragranular components, or in both. In certain embodiments, the compound is in the spray-dried particles. In certain embodiments, the compound is in the extragranular components. In certain embodiments, the compound is divided between the spray-dried particles and the extragranular components.

In yet another embodiment, provided herein is a pharmaceutical composition comprising (i) intragranular components comprising: spray-dried particles provided herein, a glidant, and a lubricant; and (ii) extragranular components comprising: a disintegrant, a filler, a glidant, a lubricant, an organic acid, and a surfactant, and (iii) a compound provided herein (e.g., Compound I, samatasvir, or a mixture thereof) in the spray-dried particles, in extragranular components, or in both. In certain embodiments, the compound is in the spray-dried particles. In certain embodiments, the compound is in the extragranular components. In certain embodiments, the compound is divided between the spray-dried particles and the extragranular components.

In still another embodiment, provided herein is a pharmaceutical composition comprising (i) intragranular components comprising: spray-dried particles provided herein, a disintegrant, a filler, a glidant, and a lubricant; and (ii) extragranular components comprising: a glidant and a lubricant, and (iii) a compound provided herein (e.g., Compound I, samatasvir, or a mixture thereof) in the spray-dried particles, in extragranular components, or in both. In certain embodiments, the compound is in the spray-dried particles. In certain embodiments, the compound is in the extragranular components. In certain embodiments, the compound is divided between the spray-dried particles and the extragranular components.

In certain embodiments, the pharmaceutical composition provided herein comprises from about 1 to about 50%, from about 2 to about 20%, or from about 5 to about 15% by weight of an intragranular disintegrant. In certain embodiments, the pharmaceutical composition provided herein comprises about 5%, about 6%, about 7%, about 8%, about 9%, about 10%, about 11%, about 12%, about 13%, about 14%, or about 15% by weight of an intragranular disintegrant. In certain embodiments, the pharmaceutical composition provided herein comprises about 6% by weight of an intragranular disintegrant. In certain embodiments, the pharmaceutical composition provided herein comprises about 10% by weight of an intragranular disintegrant. In certain embodiments, the intragranular disintegrant is a crosslinked polyvinyl pyrrolidone or croscarmellose sodium. In certain embodiments, the intragranular disintegrant is a crosslinked polyvinyl pyrrolidone. In certain embodiments, the intragranular disintegrant is POLYPLASDONE® XL. In certain embodiments, the intragranular disintegrant is croscarmellose sodium. In certain embodiments, the intragranular disintegrant is AC-DI-SOL®.

In certain embodiments, the pharmaceutical composition provided herein comprises from about 1 to about 50%, from about 2 to about 20%, or from about 5 to about 15% by weight of an extragranular disintegrant. In certain embodiments, the pharmaceutical composition provided herein comprises about 5%, about 6%, about 7%, about 8%, about 9%, about 10%, about 11%, about 12%, about 13%, about 14%, or about 15% by weight of an extragranular disintegrant. In certain embodiments, the pharmaceutical composition provided herein comprises about 6% by weight of an extragranular disintegrant. In certain embodiments, the pharmaceutical composition provided herein comprises about 10% by weight of an extragranular disintegrant. In certain embodiments, the extragranular disintegrant is a crosslinked polyvinyl pyrrolidone or croscarmellose sodium. In certain embodiments, the extragranular disintegrant is a crosslinked polyvinyl pyrrolidone. In certain embodiments, the extragranular disintegrant is POLYPLASDONE® XL. In certain embodiments, the extragranular disintegrant is croscarmellose sodium. In certain embodiments, the extragranular disintegrant is AC-DI-SOL®.

In certain embodiments, the pharmaceutical composition provided herein comprises from about 10 to about 95%, from about 20 to about 90%, from about 25 to about 75%, or from about 30 to about 70% by weight of an intragranular filler. In certain embodiments, the pharmaceutical composition provided herein comprises about 25%, about 30%, about 35%, about 40%, about 45%, about 50%, about 55%, about 60%, about 65%, about 70%, or about 75% by weight of an intragranular filler. In certain embodiments, the pharmaceutical composition provided herein comprises about 30%, about 50%, or about 65% by weight of an intragranular filler. In certain embodiments, the intragranular filler is a microcrystalline cellulose, lactose, or a mixture thereof. In certain embodiments, the intragranular filler is a microcrystalline cellulose. In certain embodiments, the intragranular filler is AVICEL® PH 102. In certain embodiments, the intragranular filler is lactose. In certain embodiments, the intragranular filler is lactose FAST FLO® 316. In certain embodiments, the intragranular filler is a mixture of microcrystalline cellulose and lactose. In certain embodiments, the intragranular filler is a mixture of AVICEL® PH 102 and lactose FAST FLO® 316.

In certain embodiments, the pharmaceutical composition provided herein comprises from about 10 to about 95%, from about 20 to about 90%, from about 25 to about 75%, or from about 30 to about 70% by weight of an extragranular filler. In certain embodiments, the pharmaceutical composition provided herein comprises about 25%, about 30%, about 35%, about 40%, about 45%, about 50%, about 55%, about 60%, about 65%, about 70%, or about 75% by weight of an extragranular filler. In certain embodiments, the pharmaceutical composition provided herein comprises about 30%, about 50%, or about 65% by weight of an extragranular filler. In certain embodiments, the extragranular filler is a microcrystalline cellulose, lactose, or a mixture thereof. In certain embodiments, the extragranular filler is a microcrystalline cellulose. In certain embodiments, the extragranular filler is AVICEL® PH 102. In certain embodiments, the extragranular filler is lactose. In certain embodiments, the extragranular filler is lactose FAST FLO® 316. In certain embodiments, the extragranular filler is a mixture of microcrystalline cellulose and lactose. In certain embodiments, the extragranular filler is a mixture of AVICEL® PH 102 and lactose FAST FLO® 316.

In certain embodiments, the pharmaceutical composition provided herein comprises from about 0.1 to about 5%, from about 0.1 to about 2%, from about 0.2 to about 1.5%, or from about 0.2 to about 1% by weight of an intragranular glidant. In certain embodiments, the pharmaceutical composition provided herein comprises about 0.1%, about 0.2%, about 0.3%, about 0.4%, about 0.5%, about 0.6%, about 0.7%, about 0.8%, about 0.9%, about 1%, about 1.1%, about 1.2%, about 1.3%, about 1.4%, or about 1.5% by weight of an intragranular glidant. In certain embodiments, the pharmaceutical composition provided herein comprises from about 0.1 to about 0.5% by weight of an intragranular glidant. In certain embodiments, the intragranular glidant is a colloidal silicon dioxide. In certain embodiment, the intragranular glidant is CAB-O-SIL®.

In certain embodiments, the pharmaceutical composition provided herein comprises from about 0.1 to about 5%, from about 0.1 to about 2%, from about 0.2 to about 1.5%, or from about 0.2 to about 1% by weight of an extragranular glidant. In certain embodiments, the pharmaceutical composition provided herein comprises about 0.1%, about 0.2%, about 0.3%, about 0.4%, about 0.5%, about 0.6%, about 0.7%, about 0.8%, about 0.9%, about 1%, about 1.1%, about 1.2%, about 1.3%, about 1.4%, or about 1.5% by weight of an extragranular glidant. In certain embodiments, the pharmaceutical composition provided herein comprises from about 0.1 to about 0.5% by weight of an extragranular glidant. In certain embodiments, the extragranular glidant is a colloidal silicon dioxide. In certain embodiment, the extragranular glidant is CAB-O-SIL®.

In certain embodiments, the pharmaceutical composition provided herein comprises from about 0.01 to about 2%, from about 0.01 to about 1%, from about 0.02 to about 0.5%, or about 0.5% to about 1% by weight of an intragranular lubricant. In certain embodiments, the pharmaceutical composition provided herein comprises about 0.05%, about 0.08%, about 0.1%, about 0.2%, about 0.3%, about 0.4%, about 0.5%, about 0.6%, about 0.7%, about 0.8%, about 0.9% or about 1% by weight of an intragranular lubricant. In certain embodiments, the pharmaceutical composition provided herein comprises from about 0.05 to about 0.25% (or about 0.5% to about 1%) by weight of an intragranular lubricant. In certain embodiments, the intragranular lubricant is magnesium stearate.

In certain embodiments, the pharmaceutical composition provided herein comprises from about 0.01 to about 2%, from about 0.01 to about 1%, from about 0.02 to about 0.5%, or about 0.5 to about 1% by weight of an extragranular lubricant. In certain embodiments, the pharmaceutical composition provided herein comprises about 0.1%, about 0.2%, about 0.3%, about 0.4%, about 0.5%, about 0.6%, about 0.7%, about 0.8%, about 0.9%, or about 1% by weight of an extragranular lubricant. In certain embodiments, the pharmaceutical composition provided herein comprises from about 0.25 to about 0.5% or about 0.5% to about 1% by weight of an extragranular lubricant. In certain embodiments, the extragranular lubricant is magnesium stearate.

In certain embodiments, the pharmaceutical composition provided herein comprises from about 1 to about 25%, from about 2 to about 20%, from about 5 to about 15%, or from about 10 to about 15% by weight of an extragranular organic acid. In certain embodiments, the pharmaceutical composition provided herein comprises about 5%, about 6%, about 7%, about 8%, about 9%, about 10%, about 11%, about 12%, about 13%, about 14%, or about 15% by weight of an extragranular organic acid. In certain embodiments, the pharmaceutical composition provided herein comprises from about 10 to about 15% by weight of an extragranular organic acid. In certain embodiments, the extragranular organic acid is tartaric acid.

In certain embodiments, the pharmaceutical composition provided herein comprises from about 1 to about 30%, from about 2 to about 25%, or from about 5 to about 25% by weight of an extragranular surfactant. In certain embodiments, the pharmaceutical composition provided herein comprises about 10%, about 11%, about 12%, about 13%, about 14%, about 15%, about 16%, about 17%, about 18%, about 19%, or about 20% by weight of an extragranular surfactant. In certain embodiments, the pharmaceutical composition provided herein comprises from about 10 to about 20% by weight of an extragranular surfactant. In certain embodiments, the extragranular surfactant is sodium lauryl sulfate.

In one embodiment, provided herein is a pharmaceutical composition comprising (i) intragranular components comprising: from about 10 to about 50% by weight of spray-dried particles provided herein, from about 0.1 to about 5% by weight of a glidant, and from about 0.05 to about 3% by weight of a lubricant; and (ii) extragranular components comprising: from about 5 to about 25% by weight of a disintegrant, from about 25 to about 80% by weight of a filler, from about 0.1 to about 5% by weight of a glidant, and from about 0.05 to about 3% by weight of a lubricant, and (iii) a compound provided herein (e.g., Compound I, samatasvir, or a mixture thereof) in the spray-dried particles, in extragranular components, or in both. In certain embodiments, the compound is in the spray-dried particles. In certain embodiments, the compound is in the extragranular components. In certain embodiments, the compound is divided between the spray-dried particles and the extragranular components.

In another embodiment, provided herein is a pharmaceutical composition comprising (i) intragranular components comprising: from about 20 to about 50% by weight of spray-dried particles provided herein, from about 0.2 to about 1.5% by weight of a glidant, and from about 0.05 to about 0.5% (or about 0.5% to about 1%) by weight of a lubricant; and (ii) extragranular components comprising: from about 5 to about 15% by weight of a disintegrant, from about 30 to about 70% by weight of a filler, from about 0.2 to about 2% by weight of a glidant, and from about 0.1 to about 1% by weight of a lubricant, and (iii) a compound provided herein (e.g., Compound I, samatasvir, or a mixture thereof) in the spray-dried particles, in extragranular components, or in both. In certain embodiments, the compound is in the spray-dried particles. In certain embodiments, the compound is in the extragranular components. In certain embodiments, the compound is divided between the spray-dried particles and the extragranular components.

In yet another embodiment, provided herein is a pharmaceutical composition comprising (i) intragranular components comprising: from about 25 to about 35% by weight of spray-dried particles provided herein, from about 0.5 to about 1.5% by weight of a glidant, and from about 0.05 to about 0.3% (or about 0.5% to about 1%) by weight of a lubricant; and (ii) extragranular components comprising: from about 6 to about 10% by weight of a disintegrant, from about 30 to about 70% by weight of a filler, about 0.5% by weight of a glidant, and from about 0.1 to about 0.5% (or about 0.5% to about 1%) by weight of a lubricant, and (iii) a compound provided herein (e.g., Compound I, samatasvir, or a mixture thereof) in the spray-dried particles, in extragranular components, or in both. In certain embodiments, the compound is in the spray-dried particles. In certain embodiments, the compound is in the extragranular components. In certain embodiments, the compound is divided between the spray-dried particles and the extragranular components.

In still another embodiment, provided herein is a pharmaceutical composition comprising (i) intragranular components comprising: from about 25 to about 35% by weight of spray-dried particles provided herein, from about 0.5 to about 1.5% by weight of a glidant, and from about 0.05 to about 0.3% (or about 0.5% to about 1%) by weight of a lubricant; and (ii) extragranular components comprising: about 10% by weight of a disintegrant, about 65% by weight of a filler, about 0.5% by weight of a glidant, and from about 0.2 to about 0.5% (or about 0.5% to about 1%) by weight of a lubricant, and (iii) a compound provided herein (e.g., Compound I, samatasvir, or a mixture thereof) in the spray-dried particles, in extragranular components, or in both. In certain embodiments, the compound is in the spray-dried particles. In certain embodiments, the compound is in the extragranular components. In certain embodiments, the compound is divided between the spray-dried particles and the extragranular components.

In one embodiment, provided herein is a pharmaceutical composition comprising (i) intragranular components comprising: from about 10 to about 50% by weight of spray-dried particles provided herein, from about 0.1 to about 5% by weight of a colloidal silicon dioxide (e.g., CAB-O-SIL® M-5P), and from about 0.05 to about 2% by weight of magnesium stearate; and (ii) extragranular components comprising: from about 5 to about 25% by weight of a polyvinyl pyrrolidone (e.g., POLYPLASDONE® XL), from about 25 to about 80% by weight of a microcrystalline cellulose (e.g., AVICEL® PH 102), from about 0.1 to about 5% by weight of a colloidal silicon dioxide (e.g., CAB-O-SIL® M-5P), and from about 0.05 to about 2% by weight of magnesium stearate, and (iii) a compound provided herein (e.g., Compound I, samatasvir, or a mixture thereof) in the spray-dried particles, in extragranular components, or in both. In certain embodiments, the compound is in the spray-dried particles. In certain embodiments, the compound is in the extragranular components. In certain embodiments, the compound is divided between the spray-dried particles and the extragranular components.

In another embodiment, provided herein is a pharmaceutical composition comprising (i) intragranular components comprising: from about 20 to about 50% by weight of spray-dried particles provided herein, from about 0.2 to about 1.5% by weight of a colloidal silicon dioxide (e.g., CAB-O-SIL® M-5P), and from about 0.05 to about 0.5% by weight of magnesium stearate; and (ii) extragranular components comprising: from about 5 to about 15% by weight of a polyvinyl pyrrolidone (e.g., POLYPLASDONE® XL), from about 30 to about 70% by weight of a microcrystalline cellulose (e.g., AVICEL® PH 102), from about 0.2 to about 2% by weight of a colloidal silicon dioxide (e.g., CAB-O-SIL® M-5P), and from about 0.1 to about 1% by weight of magnesium stearate, and (iii) a compound provided herein (e.g., Compound I, samatasvir, or a mixture thereof) in the spray-dried particles, in extragranular components, or in both. In certain embodiments, the compound is in the spray-dried particles. In certain embodiments, the compound is in the extragranular components. In certain embodiments, the compound is divided between the spray-dried particles and the extragranular components.

In yet another embodiment, provided herein is a pharmaceutical composition comprising (i) intragranular components comprising: from about 25 to about 35% by weight of spray-dried particles provided herein, from about 0.5 to about 1.5% by weight of a colloidal silicon dioxide (e.g., CAB-O-SIL® M-5P), and from about 0.05 to about 0.3% by weight of magnesium stearate; and (ii) extragranular components comprising: from about 6 to about 10% by weight of a polyvinyl pyrrolidone (e.g., POLYPLASDONE® XL), from about 30 to about 70% by weight of a microcrystalline cellulose (e.g., AVICEL® PH 102), about 0.5% by weight of a colloidal silicon dioxide (e.g., CAB-O-SIL® M-5P), and from about 0.1 to about 0.5% by weight of magnesium stearate, and (iii) a compound provided herein (e.g., Compound I, samatasvir, or a mixture thereof) in the spray-dried particles, in extragranular components, or in both. In certain embodiments, the compound is in the spray-dried particles. In certain embodiments, the compound is in the extragranular components. In certain embodiments, the compound is divided between the spray-dried particles and the extragranular components.

In still another embodiment, provided herein is a pharmaceutical composition comprising (i) intragranular components comprising: from about 25 to about 35% by weight of spray-dried particles provided herein, from about 0.5 to about 1.5% by weight a colloidal silicon dioxide (e.g., CAB-O-SIL® M-5P), and from about 0.05 to about 0.3% by weight of magnesium stearate; and (ii) extragranular components comprising: about 10% by weight of a polyvinyl pyrrolidone (e.g., POLYPLASDONE® XL), about 65% by weight of a microcrystalline cellulose (e.g., AVICEL® PH 102), about 0.5% by weight of a colloidal silicon dioxide (e.g., CAB-O-SIL® M-5P), and from about 0.2 to about 0.5% by weight of magnesium stearate, and (iii) a compound provided herein (e.g., Compound I, samatasvir, or a mixture thereof) in the spray-dried particles, in extragranular components, or in both. In certain embodiments, the compound is in the spray-dried particles. In certain embodiments, the compound is in the extragranular components. In certain embodiments, the compound is divided between the spray-dried particles and the extragranular components.

In various embodiments, the spray-dried particles are coated. In certain embodiments, the spray-dried particles are coated before becoming a part of a granular compositions described herein. In various embodiments, a compound provided herein (including Compound I) is coated. For example, Form I of Compound I can be coated before being added into any one of the pharmaceutical compositions described herein.

In certain embodiments, the pharmaceutical composition provided herein comprises from about 1 to about 1,000 mg, from about 2 to about 500 mg, from about 5 to about 200 mg, from about 5 to about 100 mg, or from about 5 to about 50 mg of Form I. In certain embodiments, the pharmaceutical composition provided herein comprises from about 1 to about 1,000 mg, from about 2 to about 500 mg, from about 5 to about 200 mg, from about 5 to about 100 mg, or from about 5 to about 50 mg of Form I, and further comprise from about 1 to about 1,000 mg, from about 2 to about 500 mg, from about 5 to about 200 mg, from about 5 to about 100 mg, from about 5 to about 50 mg, from about 5 to about 25 mg, from about 5 to about 20 mg of samatasvir.

In certain embodiments, the pharmaceutical composition provided herein comprises about 30 mg, about 35 mg, about 40 mg, about 45, about 50 mg, about 55 mg, about 60 mg, about 65 mg, about 70 mg, about 75 mg, about 80 mg, about 85 mg, about 90 mg, about 95 mg, or about 100 mg, or about 150 mg, or about 200 mg, or about 250 mg, or about 300 mg, or about 350 mg of Form I, or about 400 mg of Form I, or about 450 mg of Form, or about 500 mg of Form II. In certain embodiments, the pharmaceutical composition provided herein comprises about 30 mg, about 35 mg, about 40 mg, about 45, about 50 mg, about 55 mg, about 60 mg, about 65 mg, about 70 mg, about 75 mg, about 80 mg, about 85 mg, about 90 mg, about 95 mg, or about 100 mg, or about 150 mg, or about 200 mg, or about 250 mg, or about 300 mg, or about 400 mg of Form I, and further comprises about 30 mg, about 35 mg, about 40 mg, about 45, about 50 mg, about 55 mg, about 60 mg, about 65 mg, about 70 mg, about 75 mg, about 80 mg, about 85 mg, about 90 mg, about 95 mg, or about 100 mg of samatasvir.

In certain embodiments, the pharmaceutical composition provided herein comprises about 30 mg, about 35 mg, about 40 mg, about 45, about 50 mg, about 55 mg, about 60 mg, about 65 mg, about 70 mg, about 75 mg, about 80 mg, about 85 mg, about 90 mg, about 95 mg, or about 100 mg, or about 150 mg, or about 200 mg, or about 250 mg, or about 300 mg, or about 350 mg of Form I, or about 400 mg of Form I, or about 450 mg of Form I, or about 500 mg of Form I. In certain embodiments, the pharmaceutical composition provided herein comprises about 30 mg, about 35 mg, about 40 mg, about 45, about 50 mg, about 55 mg, about 60 mg, about 65 mg, about 70 mg, about 75 mg, about 80 mg, about 85 mg, about 90 mg, about 95 mg, or about 100 mg, or about 150 mg, or about 200 mg, or about 250 mg, or about 300 mg, or about 400 mg of Form I, and further comprises about 6 mg, about 7 mg, about 8 mg, about 9 mg, about 10 mg, about 11 mg, about 12 mg, about 13 mg, about 14, about 15 mg, about 16 mg, about 17 mg, about 18 mg, about 19 mg, about 20 mg, about 21 mg, about 22 mg, about 23 mg, about 24 mg, or about 25 mg of the samatasvir. In various embodiments, samatasvir is in Form A or an amorphous form.

In an aspect, a pharmaceutical composition is provided which comprises 50 mg samatasvir Form A and 50 mg Compound I Form I in an 800 mg tablet. In an embodiment, Compound I Form I is provided as an intragranular ingredient. In an embodiment, Compound I Form I is provided as an extragranular ingredient.

In an aspect, a pharmaceutical composition is provided which comprises 50 mg samatasvir Form A and 100 mg Compound I Form I in an 800 mg tablet. In an embodiment, Compound I Form I is provided as an intragranular ingredient. In an embodiment, Compound I Form I is provided as an extragranular ingredient.

In an aspect, a pharmaceutical composition is provided which comprises 50 mg samatasvir Form A and 200 mg Compound I Form I in an 800 mg tablet. In an embodiment, Compound I Form I is provided as an intragranular ingredient. In an embodiment, Compound I Form I is provided as an extragranular ingredient.

In certain embodiments, the pharmaceutical composition provided herein further comprises a film-coating. In certain embodiments, the film-coating is ranging from about 0.1 to about 10%, from about 0.1 to about 5%, from about 0.2 to about 5%, or from about 0.5 to about 5% by the total weight of the composition

### Other Second Therapeutic Agents

Non-limiting examples of second agents include:

HCV Protease inhibitors: Examples include Medivir HCV Protease Inhibitor (HCV-PI, TMC435, simeprevir) (Medivir/Tibotec); MK-5172 (Merck), MK-7009 (Merck), RG7227 (ITMN-191) (Roche/Pharmasset/InterMune), boceprevir (SCH 503034) (Schering), SCH 446211 (Schering), narlaprevir SCH900518 (Schering/Merck), ABT-450 (Abbott/Enanta), ACH-1625 (Achillion), BI 201335 (Boehringer Ingelheim), PHX1766 (Phenomix), VX-500 (Vertex) and telaprevir (VX-950) (Vertex).

U.S. patents disclosing protease inhibitors for the treatment of HCV include, for example, U.S. Pat. No. 6,004,933 to Spruce et al., which discloses a class of cysteine protease inhibitors for inhibiting HCV endopeptidase 2; U.S. Pat. No. 5,990,276 to Zhang et al., which discloses synthetic inhibitors of hepatitis C virus NS3 protease; U.S. Pat. No. 5,538,865 to Reyes et a; WO 02/008251 to Corvas International, Inc., and U.S. Pat. No. 7,169,760, US2005/176648, WO 02/08187 and WO 02/008256 to Schering Corporation. HCV inhibitor tripeptides are disclosed in U.S. Pat. Nos. 6,534,523, 6,410,531, and 6,420,380 to Boehringer Ingelheim and WO 02/060926 to Bristol Myers Squibb. Diaryl peptides as NS3 serine protease inhibitors of HCV are disclosed in WO 02/48172 and U.S. Pat. No. 6,911,428 to Schering Corporation. Imidazoleidinones as NS3 serine protease inhibitors of HCV are disclosed in WO 02/08198 and U.S. Pat. No. 6,838,475 to Schering Corporation and WO 02/48157 and U.S. Pat. No. 6,727,366 to Bristol Myers Squibb. WO 98/17679 and U.S. Pat. No. 6,265,380 to Vertex Pharmaceuticals and WO 02/48116 and U.S. Pat. No. 6,653,295 to Bristol Myers Squibb also disclose HCV protease inhibitors. Further examples of HCV serine protease inhibitors are provided in U.S. Pat. No. 6,872,805 (Bristol-Myers Squibb); WO 2006000085 (Boehringer Ingelheim); U.S. Pat. No. 7,208,600 (Vertex); US 2006/0046956 (Schering-Plough); WO 2007/001406 (Chiron); US 2005/0153877; WO 2006/119061 (Merck); WO 00/09543 (Boehringer Ingelheim), U.S. Pat. No. 6,323,180 (Boehringer Ingelheim) WO 03/064456 (Boehringer Ingelheim), U.S. Pat. No. 6,642,204 (Boehringer Ingelheim), WO 03/064416 (Boehringer Ingelheim), U.S. Pat. No. 7,091,184 (Boehringer Ingelheim), WO 03/053349 (Bristol-Myers Squibb), U.S. Pat. No. 6,867,185, WO 03/099316 (Bristol-Myers Squibb), U.S. Pat. No. 6,869,964, WO 03/099274 (Bristol-Myers Squibb), U.S. Pat. No. 6,995,174, WO 2004/032827 (Bristol-Myers Squibb), U.S. Pat. No. 7,041,698, WO 2004/043339 and U.S. Pat. No. 6,878,722 (Bristol-Myers Squibb);

HCV polymerase inhibitors, including nucleoside and non-nucleoside polymerase inhibitors, such as ribavirin, viramidine, clemizole, filibuvir (PF-00868554), HCV POL, NM 283 (valopicitabine), MK-0608, 7-Fluoro-MK-0608, MK-3281, IDX-375, ABT-072, ABT-333, ANA598, BI 207127, GS 9190, PSI-6130, R1626, PSI-6206, PSI-938, PSI-7851, PSI-7977 (GS-7977, sofosbuvir, Sovaldi), RG1479, RG7128, HCV-796 VCH-759 or VCH-916.

Interfering RNA (iRNA) based antivirals, including short interfering RNA (siRNA) based antivirals, such as Sima-034 and others described in International Patent Publication Nos. WO/03/070750 and WO 2005/012525, and US Patent Publication No. US 2004/0209831;

Antisense phosphorothioate oligodeoxynucleotides (S-ODN) complementary to sequence stretches in the 5′ non-coding region (NCR) of the virus (Alt M. et al., Hepatology, 1995, 22, 707-717), or nucleotides 326-348 comprising the 3′ end of the NCR and nucleotides 371-388 located in the core coding region of the HCV RNA (Alt M. et al., Archives of Virology, 1997, 142, 589-599; Galderisi U. et al., Journal of Cellular Physiology, 1999, 181, 251-257);

Inhibitors of IRES-dependent translation (Ikeda N et al., Agent for the prevention and treatment of hepatitis C, Japanese Patent Pub. JP-08268890; Kai Y. et al., Prevention and treatment of viral diseases, Japanese Patent Pub. JP-10101591);

HCV NS5A inhibitors, such as BMS-790052 (daclatasvir, Bristol-Myers Squibb), PPI-461 (Presidio Pharmaceuticals), PPI-1301 (Presidio Pharmaceuticals), samatasvir (IDX719, Idenix Pharmaceuticals), AZD7295 (Arrow Therapeutics, AstraZeneca), EDP-239 (Enanta), ACH-2928 (Achillion), ACH-3102 (Achillion), ABT-267 (Abbott), or GS-5885 (Gilead);

HCV entry inhibitors, such as celgosivir (MK-3253) (MIGENIX Inc.), SP-30 (Samaritan Pharmaceuticals), ITX4520 (iTherX), ITX5061 (iTherX), PRO-206 (Progenics Pharmaceuticals) and other entry inhibitors by Progenics Pharmaceuticals, e.g., as disclosed in U.S. Patent Publication No. 2006/0198855;

Ribozymes, such as nuclease-resistant ribozymes (Maccjak, D. J. et al., Hepatology 1999, 30, abstract 995) and those disclosed in U.S. Pat. No. 6,043,077 to Barber et al., and U.S. Pat. Nos. 5,869,253 and 5,610,054 to Draper et al.; and

In certain embodiments, the compounds provided herein can be administered in combination with any of the compounds described by Idenix Pharmaceuticals in International Publication Nos. WO 01/90121, WO 01/92282, WO 2004/003000, WO 2004/002422 and WO 2004/002999.

In certain embodiments, Compound I Form I, or a composition comprising Compound I Form I, is administered in combination or alternation with a second anti-viral agent including an interferon, a nucleotide analogue, a polymerase inhibitor, an NS3 protease inhibitor, an NS5A inhibitor, an entry inhibitor, a non-nucleoside polymerase inhibitor, a cyclosporine immune inhibitor, an NS4A antagonist, an NS4B-RNA binding inhibitor, a locked nucleic acid mRNA inhibitor, a cyclophilin inhibitor, or a combination thereof.

### Exemplary Second Therapeutic Agents for Treatment of HCV

In certain embodiments, one or more compounds provided herein can be administered in combination or alternation with an anti-hepatitis C virus polymerase inhibitor, such as ribavirin, viramidine, HCV POL, NM 283 (valopicitabine), MK-0608, 7-Fluoro-MK-0608, PSI-6130, R1626, PSI-6206, PSI-938, R1479, HCV-796, VX-950 (Telaprevir, Vertex), GS 9190 NN (Gilead), GS 9256 (Gilead), PSI-7792 (BMS), BI 207127 (BI), R7128 (Roche), PSI-7977 (GS-7977, sofosbuvir, Sovaldi) (Gilead-Pharmasset), PSI-938 (Pharmasset), VX-222 (Vertex), ALS-2200 (Vertex), ALS-2158 (Vertex), MK-0608 (Merck), TMC649128 (Medivir), PF-868554 (Pfizer), PF-4878691 (Pfizer), ANA598 (Roche), VCH-759 (Vertex), IDX184 (Idenix), IDX375 (Idenix), A-837093 (Abbott), GS 9190 (Gilead), GSK625433 (GlaxoSmithKline), ABT-072 (Abbott), ABT-333 (Abbott), INX-189 (Inhibitex), or EDP-239 (Enanta).

In certain embodiments, the one or more compounds provided herein can be administered in combination with ribavarin and an anti-hepatitis C virus interferon, such as Intron A® (interferon alfa-2b) and Pegasys® (Peginterferon alfa-2a); Roferon A® (Recombinant interferon alfa-2a), Infergen® (consensus interferon; interferon alfacon-1), PEG-Intron® (pegylated interferon alfa-2b), Zalbin (albinterferon alfa-2b), omega interferon, pegylated interferon lambda, and Pegasys® (pegylated interferon alfa-2a), or combinations thereof.

In certain embodiments, one or more compounds provided herein can be administered in combination or alternation with an anti-hepatitis C virus protease inhibitor such as ITMN-191, SCH 503034 (bocepravir), VX950 (telaprevir), VX985, VX500, VX813, PHX1766, BMS-650032, GS 9256, BI 201335, IDX320, R7227, MK-5172 (Merck), MK-7009 (vaniprevir), TMC435 (simeprevir), BMS-791325, ACH-1625, ACH-2684, ABT-450, or AVL-181, or combinations thereof.

In certain embodiments, one or more compounds provided herein can be administered in combination or alternation with an HCV NS5A inhibitor, such as BMS-790052 (daclatasvir, Bristol-Myers Squibb), PPI-461 (Presidio Pharmaceuticals), PPI-1301 (Presidio Pharmaceuticals), AZD7295 (Arrow Therapeutics, AstraZeneca), EDP-239 (Enanta), ACH-2928 (Achillion), ACH-3102 (Achillion), ABT-267 (Abbott), or GS-5885 (Gilead), or combinations thereof.

In certain embodiments, one or more compounds provided herein can be administered in combination or alternation with an anti-hepatitis C virus vaccine, such as TG4040, PeviPROTM, CGI-5005, HCV/MF59, GV1001, IC41, GNI-103, GenPhar HCV vaccine, C-Vaxin, CSL123, Hepavaxx C, ChronVac-C® or INNO0101 (E1), or combinations thereof.

In certain embodiments, one or more compounds provided herein can be administered in combination or alternation with an anti-hepatitis C virus monoclonal antibody, such as MBL-HCV1, AB68 or XTL-6865 (formerly HepX-C); or an anti-hepatitis C virus polyclonal antibody, such as cicavir, or combinations thereof.

In certain embodiments, one or more compounds provided herein can be administered in combination or alternation with an anti-hepatitis C virus immunomodulator, such as Zadaxin® (thymalfasin), SCV-07, NOV-205, or Oglufanide, or combinations thereof.

In certain embodiments, one or more compounds provided herein can be administered in combination or alternation with cyclophilin inhibitor, such as Enanta cyclophilin binder, SCY-635, or Debio-025, or combinations thereof.

In certain embodiments, one or more compounds provided herein can be administered in combination or alternation with Nexavar, doxorubicin, PI-88, amantadine, JBK-122, VGX-410C, MX-3253 (Ceglosivir), Suvus (BIVN-401 or virostat), PF-03491390 (formerly IDN-6556), G126270, UT-231B, DEBIO-025, EMZ702, ACH-0137171, MitoQ, ANA975, AVI-4065, Bavituxinab (Tarvacin), Alinia (nitrazoxanide) or PYN17, or combinations thereof.

In certain embodiments, one or more compounds provided herein can be administered in combination or alternation with simeprevir, sofosbuvir, telaprevir, bocepravir, interferon alfacon-1, interferon alfa-2b, pegylated interferon alpha 2a, pegylated interferon alpha 2b, ribavirin, or combinations thereof.

In certain embodiments, one or more compounds provided herein can be administered in combination or alternation with a protease inhibitor. In certain embodiments, one or more compounds provided herein can be administered in combination or alternation with telaprevir. In certain embodiments, one or more compounds provided herein can be administered in combination or alternation with bocepravir.

In certain embodiments, one or more compounds provided herein can be administered in combination or alternation with a protease inhibitor and in combination or alternation with ribavirin. In certain embodiments, one or more compounds provided herein can be administered in combination or alternation with telaprevir and in combination or alternation with ribavirin. In certain embodiments, one or more compounds provided herein can be administered in combination or alternation with bocepravir and in combination or alternation with ribavirin.

In certain embodiments, one or more compounds provided herein can be administered in combination or alternation with a protease inhibitor and not in combination or alternation with ribavirin. In certain embodiments, one or more compounds provided herein can be administered in combination or alternation with telaprevir and not in combination or alternation with ribavirin. In certain embodiments, one or more compounds provided herein can be administered in combination or alternation with bocepravir and not in combination or alternation with ribavirin.

In certain embodiments, one or more compounds provided herein can be administered in combination or alternation with an interferon. In certain embodiments, one or more compounds provided herein can be administered in combination or alternation with interferon alfacon-1. In certain embodiments, one or more compounds provided herein can be administered in combination or alternation with interferon alfa-2b. In certain embodiments, one or more compounds provided herein can be administered in combination or alternation with pegylated interferon alpha 2a. In certain embodiments, one or more compounds provided herein can be administered in combination or alternation with pegylated interferon alpha 2b.

In certain embodiments, one or more compounds provided herein can be administered in combination or alternation with an interferon and in combination or alternation with ribavirin. In certain embodiments, one or more compounds provided herein can be administered in combination or alternation with interferon alfacon-1 and in combination or alternation with ribavirin. In certain embodiments, one or more compounds provided herein can be administered in combination or alternation with interferon alfa-2b and in combination or alternation with ribavirin. In certain embodiments, one or more compounds provided herein can be administered in combination or alternation with pegylated interferon alpha 2a and in combination or alternation with ribavirin. In certain embodiments, one or more compounds provided herein can be administered in combination or alternation with pegylated interferon alpha 2b and in combination or alternation with ribavirin.

In certain embodiments, one or more compounds can be administered in combination or alternation with one or more of the second agents provided herein and not in combination or alternation with ribavirin. In certain embodiments, one or more compounds provided herein can be administered in combination or alternation with an interferon and not in combination or alternation with ribavirin. In certain embodiments, one or more compounds provided herein can be administered in combination or alternation with interferon alfacon-1 and not in combination or alternation with ribavirin. In certain embodiments, one or more compounds provided herein can be administered in combination or alternation with interferon alfa-2b and not in combination or alternation with ribavirin. In certain embodiments, one or more compounds provided herein can be administered in combination or alternation with pegylated interferon alpha 2a and not in combination or alternation with ribavirin. In certain embodiments, one or more compounds provided herein can be administered in combination or alternation with pegylated interferon alpha 2b and not in combination or alternation with ribavirin.

In certain embodiments, the second agent can be formulated or packaged with the compound provided herein. Of course, the second agent will only be formulated with the compound provided herein when, according to the judgment of those of skill in the art, such co-formulation should not interfere with the activity of either agent or the method of administration. In certain embodiments, the compound provided herein and the second agent are formulated separately. They can be packaged together, or packaged separately, for the convenience of the practitioner of skill in the art.

### Methods of Use

Provided herein also are methods of using Compound I which includes use of one or more solid forms of Compound I. For example, a method can include administering a therapeutically effective amount of one or more solid forms of Compound I, or a composition provided herein, by a suitable route of administration.

The methods provided herein encompass administering pharmaceutical compositions containing at least one compound as described herein, including a solid form of Compound I (including a crystalline form, for example, Form I, II, or III), if appropriate in the salt form, either used alone or in the form of a combination with one or more compatible and pharmaceutically acceptable carriers, such as diluents or adjuvants, or with another anti-HCV agent.

In clinical practice the active agents provided herein may be administered by any conventional route, in particular orally, parenterally, rectally or by inhalation (e.g. in the form of aerosols). In certain embodiments, the compound provided herein is administered orally.

Use may be made, as solid compositions for oral administration, of tablets, pills, hard gelatin capsules, powders or granules. In these compositions, the active product is mixed with one or more inert diluents or adjuvants, such as sucrose, lactose, or starch.

These compositions can comprise substances other than diluents, for example a lubricant, such as magnesium stearate, or a coating intended for controlled release.

Use may be made, as liquid compositions for oral administration, of solutions which are pharmaceutically acceptable, suspensions, emulsions, syrups, and elixirs containing inert diluents, such as water or liquid paraffin. These compositions can also comprise substances other than diluents, for example wetting, sweetening, or flavoring products.

The compositions for parenteral administration can be emulsions or sterile solutions. Use may be made, as solvent or vehicle, of propylene glycol, a polyethylene glycol, vegetable oils, in particular olive oil, or injectable organic esters, for example ethyl oleate. These compositions can also contain adjuvants, in particular wetting, isotonizing, emulsifying, dispersing, and stabilizing agents. Sterilization can be carried out in several ways, for example using a bacteriological filter, by radiation or by heating. They can also be prepared in the form of sterile solid compositions which can be dissolved at the time of use in sterile water or any other injectable sterile medium.

The compositions for rectal administration are suppositories or rectal capsules which contain, in addition to the active principle, excipients such as cocoa butter, semi-synthetic glycerides, or polyethylene glycols.

In certain embodiments, a composition provided herein is a pharmaceutical composition or a single unit dosage form. Pharmaceutical compositions and single unit dosage forms provided herein comprise a prophylactically or therapeutically effective amount of one or more prophylactic or therapeutic agents (e.g., a compound provided herein, or other prophylactic or therapeutic agent), and a typically one or more pharmaceutically acceptable carriers. The term “carrier” includes a diluent, adjuvant (e.g., Freund's adjuvant (complete and incomplete)), excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water can be used as a carrier when the pharmaceutical composition is administered intravenously. Saline solutions and aqueous dextrose and glycerol solutions can also be employed as liquid carriers, particularly for injectable solutions. Examples of suitable pharmaceutical carriers are described in “Remington's Pharmaceutical Sciences” by E. W. Martin.

Typical pharmaceutical compositions and dosage forms comprise one or more excipients. Suitable excipients are well-known to those skilled in the art of pharmacy, and non-limiting examples of suitable excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol, and the like. Whether a particular excipient is suitable for incorporation into a pharmaceutical composition or dosage form depends on a variety of factors well known in the art including, but not limited to, the way in which the dosage form will be administered to a subject and the specific active ingredients in the dosage form. The composition or single unit dosage form, if desired, can also contain minor amounts of wetting or emulsifying agents, or pH buffering agents.

Lactose free compositions provided herein can comprise excipients that are well known in the art and are listed, for example, in the U.S. Pharmocopia (USP) SP (XXI)/NF (XVI). In general, lactose free compositions comprise an active ingredient, a binder/filler, and a lubricant in pharmaceutically compatible and pharmaceutically acceptable amounts. Exemplary lactose free dosage forms comprise an active ingredient, microcrystalline cellulose, pre gelatinized starch, and magnesium stearate.

Further encompassed herein are anhydrous pharmaceutical compositions and dosage forms comprising active ingredients, since water can facilitate the degradation of some compounds. For example, the addition of water (e.g., 5%) is widely accepted in the pharmaceutical arts as a means of simulating long term storage in order to determine characteristics such as shelf life or the stability of formulations over time. See, e.g., Jens T. Carstensen, Drug Stability: Principles & Practice, 2d. Ed., Marcel Dekker, New York, 1995, pp. 379 80. In effect, water and heat accelerate the decomposition of some compounds. Thus, the effect of water on a formulation can be of great significance since moisture and/or humidity are commonly encountered during manufacture, handling, packaging, storage, shipment, and use of formulations.

Anhydrous pharmaceutical compositions and dosage forms provided herein can be prepared using anhydrous or low moisture containing ingredients and low moisture or low humidity conditions. Pharmaceutical compositions and dosage forms that comprise lactose and at least one active ingredient that comprises a primary or secondary amine can be anhydrous if substantial contact with moisture and/or humidity during manufacturing, packaging, and/or storage is expected.

An anhydrous pharmaceutical composition should be prepared and stored such that its anhydrous nature is maintained. Accordingly, anhydrous compositions can be packaged using materials known to prevent exposure to water such that they can be included in suitable formulary kits. Examples of suitable packaging include, but are not limited to, hermetically sealed foils, plastics, unit dose containers (e.g., vials), blister packs, and strip packs.

Further provided are pharmaceutical compositions and dosage forms that comprise one or more compounds that reduce the rate by which an active ingredient will decompose. Such compounds, which are referred to herein as “stabilizers,” include, but are not limited to, antioxidants such as ascorbic acid, pH buffers, or salt buffers.

The pharmaceutical compositions and single unit dosage forms can take the form of solutions, suspensions, emulsion, tablets, pills, capsules, powders, sustained-release formulations and the like. Oral formulation can include standard carriers such as pharmaceutical grades of mannitol, lactose, starch, magnesium stearate, sodium saccharine, cellulose, magnesium carbonate, etc. Such compositions and dosage forms will contain a prophylactically or therapeutically effective amount of a prophylactic or therapeutic agent, in certain embodiments, in purified form, together with a suitable amount of carrier so as to provide the form for proper administration to the subject. The formulation should suit the mode of administration. In a certain embodiment, the pharmaceutical compositions or single unit dosage forms are sterile and in suitable form for administration to a subject, for example, an animal subject, such as a mammalian subject, for example, a human subject.

A pharmaceutical composition is formulated to be compatible with its intended route of administration. Examples of routes of administration include, but are not limited to, parenteral, e.g., intravenous, intradermal, subcutaneous, intramuscular, subcutaneous, oral, buccal, sublingual, inhalation, intranasal, transdermal, topical, transmucosal, intra-tumoral, intra-synovial, and rectal administration. In a specific embodiment, the composition is formulated in accordance with routine procedures as a pharmaceutical composition adapted for intravenous, subcutaneous, intramuscular, oral, intranasal, or topical administration to human beings. In an embodiment, a pharmaceutical composition is formulated in accordance with routine procedures for subcutaneous administration to human beings. Typically, compositions for intravenous administration are solutions in sterile isotonic aqueous buffer. Where necessary, the composition may also include a solubilizing agent and a local anesthetic such as lignocamne to ease pain at the site of the injection.

Examples of dosage forms include, but are not limited to: tablets; caplets; capsules, such as soft elastic gelatin capsules; cachets; troches; lozenges; dispersions; suppositories; ointments; cataplasms (poultices); pastes; powders; dressings; creams; plasters; solutions; patches; aerosols (e.g., nasal sprays or inhalers); gels; liquid dosage forms suitable for oral or mucosal administration to a subject, including suspensions (e.g., aqueous or non-aqueous liquid suspensions, oil in water emulsions, or a water in oil liquid emulsions), solutions, and elixirs; liquid dosage forms suitable for parenteral administration to a subject; and sterile solids (e.g., crystalline or amorphous solids) that can be reconstituted to provide liquid dosage forms suitable for parenteral administration to a subject.

The composition, shape, and type of dosage forms provided herein will typically vary depending on their use. For example, a dosage form used in the initial treatment of viral infection may contain larger amounts of one or more of the active ingredients it comprises than a dosage form used in the maintenance treatment of the same infection. Similarly, a parenteral dosage form may contain smaller amounts of one or more of the active ingredients it comprises than an oral dosage form used to treat the same disease or disorder. These and other ways in which specific dosage forms encompassed herein will vary from one another will be readily apparent to those skilled in the art. See, e.g., Remington's Pharmaceutical Sciences, 20th ed., Mack Publishing, Easton Pa. (2000).

Generally, the ingredients of compositions are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

Typical dosage forms comprise a compound provided herein, or a pharmaceutically acceptable salt, solvate or hydrate thereof lie within the range of from about 0.1 mg to about 1000 mg per day, given as a single once-a-day dose in the morning or as divided doses throughout the day taken with food. Particular dosage forms can have about 0.1, 0.2, 0.3, 0.4, 0.5, 1.0, 2.0, 2.5, 5.0, 10.0, 15.0, 20.0, 25.0, 50.0, 100, 200, 250, 300, 350, 400, 450, 500, or 1000 mg of the active compound.

In human therapeutics, the doctor will determine the posology which he considers most appropriate according to a preventive or curative treatment and according to the age, weight, stage of the infection and other factors specific to the subject to be treated. In certain embodiments, doses are from about 10 to about 1000 mg Compound I or equivalent per day, from about 40 to about 900 mg Compound I or equivalent per day, from about 60 to about 800 mg Compound I or equivalent per day, from about 80 to about 700 Compound I or equivalent mg per day, from about 100 to about 600 mg Compound I or equivalent per day, from about 150 to about 500 mg Compound I or equivalent per day, from about 200 to about 400 mg Compound I or equivalent per day, or from about 250 to 350 mg Compound I or equivalent per day for an adult, about 350 to 450 mg Compound I or equivalent per day for an adult. In certain embodiments, doses are from about 100 to about 600 mg Compound I or equivalent per day per adult. In certain embodiments, doses are no less than 50 mg Compound I or equivalent per day, no less than 100 mg Compound I or equivalent per day, no less than about 150 mg Compound I or equivalent per day, no less than 200 mg Compound I or equivalent per day, no less than 300 mg Compound I or equivalent per day per adult, no less than 450 mg Compound I or equivalent per day per adult. In particular embodiments, the dosages are selected from about 50 mg, 100 mg, 150 mg, 200 mg, 300 mg, 350 mg, 400 mg, 450 mg, 500 mg, or 600 mg Compound I or equivalent per day per adult. For example, the dose is about 300 mg Compound I or equivalent per day per adult, or the dose is about 450 mg Compound I or equivalent per day per adult.

### Oral Dosage Forms

Pharmaceutical compositions that are suitable for oral administration can be presented as discrete dosage forms, such as, but are not limited to, tablets (e.g., chewable tablets), caplets, capsules, and liquids (e.g., flavored syrups). Such dosage forms contain predetermined amounts of active ingredients, and may be prepared by methods of pharmacy well known to those skilled in the art. See generally, Remington's Pharmaceutical Sciences, 20th ed., Mack Publishing, Easton Pa. (2000).

In certain embodiments, the oral dosage forms are solid and prepared under anhydrous conditions with anhydrous ingredients, as described in detail herein. However, the scope of the compositions provided herein extends beyond anhydrous, solid oral dosage forms. As such, further forms are described herein.

Typical oral dosage forms are prepared by combining the active ingredient(s) in an intimate admixture with at least one excipient according to conventional pharmaceutical compounding techniques. Excipients can take a wide variety of forms depending on the form of preparation desired for administration. For example, excipients suitable for use in oral liquid or aerosol dosage forms include, but are not limited to, water, glycols, oils, alcohols, flavoring agents, preservatives, and coloring agents. Examples of excipients suitable for use in solid oral dosage forms (e.g., powders, tablets, capsules, and caplets) include, but are not limited to, starches, sugars, micro crystalline cellulose, diluents, granulating agents, lubricants, binders, and disintegrating agents.

Because of their ease of administration, tablets and capsules represent the most advantageous oral dosage unit forms, in which case solid excipients are employed. If desired, tablets can be coated by standard aqueous or non-aqueous techniques. Such dosage forms can be prepared by any of the methods of pharmacy. In general, pharmaceutical compositions and dosage forms are prepared by uniformly and intimately admixing the active ingredients with liquid carriers, finely divided solid carriers, or both, and then shaping the product into the desired presentation if necessary.

For example, a tablet can be prepared by compression or molding. Compressed tablets can be prepared by compressing in a suitable machine the active ingredients in a free flowing form such as powder or granules, optionally mixed with an excipient. Molded tablets can be made by molding in a suitable machine a mixture of the powdered compound moistened with an inert liquid diluent.

In various embodiments, at least one of the compositions described herein is in an oral dosage form. For example, at least one of the compositions is tablets or suspensions. In some embodiments, at least one of the compositions is tablets. In certain embodiments, provided herein are tablets, each comprising a pharmaceutical composition including Compound I and a pharmaceutically acceptable excipient. In particular embodiments, the tablets each comprises a pharmaceutical composition including Form I of Compound I and a pharmaceutically acceptable excipient. In certain embodiments, provided herein are tablets, each comprising a pharmaceutical composition including Compound I, a second therapeutic agent, and a pharmaceutically acceptable excipient. In particular embodiments, the tablets each comprises a pharmaceutical composition including Form I of Compound I, a second therapeutic agent, and a pharmaceutically acceptable excipient. In one embodiment, the tablets each comprises a pharmaceutical composition including Form I of Compound I, samatasvir, and a pharmaceutically acceptable excipient.

In certain embodiments, the oral dosage forms are solid and prepared under anhydrous conditions with anhydrous ingredients, as described in detail herein. However, the scope of the compositions provided herein extends beyond anhydrous, solid oral dosage forms. As such, further forms are described herein.

Typical oral dosage forms are prepared by combining the active ingredient(s) in an intimate admixture with at least one excipient according to conventional pharmaceutical compounding techniques. Excipients can take a wide variety of forms depending on the form of preparation desired for administration. For example, excipients suitable for use in oral liquid or aerosol dosage forms include, but are not limited to, water, glycols, oils, alcohols, flavoring agents, preservatives, and coloring agents. Examples of excipients suitable for use in solid oral dosage forms (e.g., powders, tablets, capsules, and caplets) include, but are not limited to, starches, sugars, micro crystalline cellulose, diluents, granulating agents, lubricants, binders, and disintegrating agents.

Because of their ease of administration, tablets and capsules represent the most advantageous oral dosage unit forms, in which case solid excipients are employed. If desired, tablets can be coated by standard aqueous or non-aqueous techniques. Such dosage forms can be prepared by any of the methods of pharmacy. In general, pharmaceutical compositions and dosage forms are prepared by uniformly and intimately admixing the active ingredients with liquid carriers, finely divided solid carriers, or both, and then shaping the product into the desired presentation if necessary.

For example, a tablet can be prepared by compression or molding. Compressed tablets can be prepared by compressing in a suitable machine the active ingredients in a free flowing form such as powder or granules, optionally mixed with an excipient. Molded tablets can be made by molding in a suitable machine a mixture of the powdered compound moistened with an inert liquid diluent.

In various embodiments, at least one of the compositions described herein is in an oral dosage form. For example, at least one of the compositions is tablets, capsules, or suspensions.

In some embodiments, at least one of the compositions is capsules. In certain embodiments, provided herein are capsules, each comprising a pharmaceutical composition including Compound I or equivalent and a pharmaceutically acceptable excipient. In particular embodiments, the capsules each comprises a pharmaceutical composition including Compound I or equivalent and a pharmaceutically acceptable excipient. In certain embodiments, provided herein are capsules, each comprising a pharmaceutical composition including Compound I or equivalent, a second therapeutic agent, and a pharmaceutically acceptable excipient. In one embodiment, the capsules each comprises a pharmaceutical composition including Compound I or equivalent, samatasvir, and a pharmaceutically acceptable excipient.

In some embodiments, at least one of the compositions is tablets. In certain embodiments, provided herein are tablets, each comprising a pharmaceutical composition including Compound I and a pharmaceutically acceptable excipient. In particular embodiments, the tablets each comprises a pharmaceutical composition including Compound I or equivalent and a pharmaceutically acceptable excipient. In certain embodiments, provided herein are tablets, each comprising a pharmaceutical composition including Compound I or equivalent, a second therapeutic agent, and a pharmaceutically acceptable excipient. In one embodiment, the tablets each comprises a pharmaceutical composition including Compound I or equivalent, samatasvir, and a pharmaceutically acceptable excipient.

### Parenteral Dosage Forms

In certain embodiments, provided are parenteral dosage forms. Parenteral dosage forms can be administered to subjects by various routes including, but not limited to, subcutaneous, intravenous (including bolus injection), intramuscular, and intraarterial. Because their administration typically bypasses subjects' natural defenses against contaminants, parenteral dosage forms are typically, sterile or capable of being sterilized prior to administration to a subject. Examples of parenteral dosage forms include, but are not limited to, solutions ready for injection, dry products ready to be dissolved or suspended in a pharmaceutically acceptable vehicle for injection, suspensions ready for injection, and emulsions.

Suitable vehicles that can be used to provide parenteral dosage forms are well known to those skilled in the art. Examples include, but are not limited to: Water for Injection USP; aqueous vehicles such as, but not limited to, Sodium Chloride Injection, Ringer's Injection, Dextrose Injection, Dextrose and Sodium Chloride Injection, and Lactated Ringer's Injection; water miscible vehicles such as, but not limited to, ethyl alcohol, polyethylene glycol, and polypropylene glycol; and non-aqueous vehicles such as, but not limited to, corn oil, cottonseed oil, peanut oil, sesame oil, ethyl oleate, isopropyl myristate, and benzyl benzoate.

Compounds that increase the solubility of one or more of the active ingredients disclosed herein can also be incorporated into the parenteral dosage forms.

### Transdermal, Topical & Mucosal Dosage Forms

Also provided are transdermal, topical, and mucosal dosage forms. Transdermal, topical, and mucosal dosage forms include, but are not limited to, ophthalmic solutions, sprays, aerosols, creams, lotions, ointments, gels, solutions, emulsions, suspensions, or other forms known to one of skill in the art. See, e.g., Remington's Pharmaceutical Sciences, 16^{th}, 18th and 20^{th} eds., Mack Publishing, Easton Pa. (1980, 1990 & 2000); and Introduction to Pharmaceutical Dosage Forms, 4th ed., Lea & Febiger, Philadelphia (1985). Dosage forms suitable for treating mucosal tissues within the oral cavity can be formulated as mouthwashes or as oral gels. Further, transdermal dosage forms include “reservoir type” or “matrix type” patches, which can be applied to the skin and worn for a specific period of time to permit the penetration of a desired amount of active ingredients.

Suitable excipients (e.g., carriers and diluents) and other materials that can be used to provide transdermal, topical, and mucosal dosage forms encompassed herein are well known to those skilled in the pharmaceutical arts, and depend on the particular tissue to which a given pharmaceutical composition or dosage form will be applied. With that fact in mind, typical excipients include, but are not limited to, water, acetone, ethanol, ethylene glycol, propylene glycol, butane 1,3 diol, isopropyl myristate, isopropyl palmitate, mineral oil, and mixtures thereof to form lotions, tinctures, creams, emulsions, gels or ointments, which are nontoxic and pharmaceutically acceptable. Moisturizers or humectants can also be added to pharmaceutical compositions and dosage forms if desired. Examples of such additional ingredients are well known in the art. See, e.g., Remington's Pharmaceutical Sciences, 16^{th}, 18th and 20^{th} eds., Mack Publishing, Easton Pa. (1980, 1990 & 2000).

Depending on the specific tissue to be treated, additional components may be used prior to, in conjunction with, or subsequent to treatment with active ingredients provided. For example, penetration enhancers can be used to assist in delivering the active ingredients to the tissue. Suitable penetration enhancers include, but are not limited to: acetone; various alcohols such as ethanol, oleyl, and tetrahydrofuryl; alkyl sulfoxides such as dimethyl sulfoxide; dimethyl acetamide; dimethyl formamide; polyethylene glycol; pyrrolidones such as polyvinylpyrrolidone; Kollidon grades (Povidone, Polyvidone); urea; and various water soluble or insoluble sugar esters such as Tween 80 (polysorbate 80) and Span 60 (sorbitan monostearate).

The pH of a pharmaceutical composition or dosage form, or of the tissue to which the pharmaceutical composition or dosage form is applied, may also be adjusted to improve delivery of one or more active ingredients. Similarly, the polarity of a solvent carrier, its ionic strength, or tonicity can be adjusted to improve delivery. Compounds such as stearates can also be added to pharmaceutical compositions or dosage forms to advantageously alter the hydrophilicity or lipophilicity of one or more active ingredients so as to improve delivery. In this regard, stearates can serve as a lipid vehicle for the formulation, as an emulsifying agent or surfactant, and as a delivery enhancing or penetration enhancing agent. Different salts, hydrates, or solvates of the active ingredients can be used to further adjust the properties of the resulting composition.

### Dosage and Unit Dosage Forms

In human therapeutics, the doctor will determine the posology which he considers most appropriate according to a preventive or curative treatment and according to the age, weight, stage of the infection and other factors specific to the subject to be treated. In certain embodiments, doses are from about 1 to about 1000 mg per day for an adult, or from about 5 to about 250 mg per day or from about 10 to 50 mg per day for an adult. In certain embodiments, doses are from about 5 to about 400 mg per day or 25 to 200 mg per day per adult. In certain embodiments, dose rates of from about 50 to about 500 mg per day are also contemplated.

In further aspects, provided are methods of treating or preventing an HCV infection in a subject by administering, to a subject in need thereof, an effective amount of a compound provided herein, or a pharmaceutically acceptable salt thereof. The amount of the compound or composition which will be effective in the prevention or treatment of a disorder or one or more symptoms thereof will vary with the nature and severity of the disease or condition, and the route by which the active ingredient is administered. The frequency and dosage will also vary according to factors specific for each subject depending on the specific therapy (e.g., therapeutic or prophylactic agents) administered, the severity of the disorder, disease, or condition, the route of administration, as well as age, body, weight, response, and the past medical history of the subject. Effective doses may be extrapolated from dose-response curves derived from in vitro or animal model test systems.

In certain embodiments, exemplary doses of a composition include milligram or microgram amounts of the active compound per kilogram of subject or sample weight (e.g., about 10 micrograms per kilogram to about 50 milligrams per kilogram, about 100 micrograms per kilogram to about 25 milligrams per kilogram, or about 100 microgram per kilogram to about 10 milligrams per kilogram). For compositions provided herein, in certain embodiments, the dosage administered to a subject is 0.140 mg/kg to 3 mg/kg of the subject's body weight, based on weight of the active compound. In certain embodiments, the dosage administered to a subject is between 0.20 mg/kg and 2.00 mg/kg, or between 0.30 mg/kg and 1.50 mg/kg of the subject's body weight.

In certain embodiments, a dose of a compound or composition provided herein can be administered to achieve a steady-state concentration of the active ingredient in blood or serum of the subject. The steady-state concentration can be determined by measurement according to techniques available to those of skill or can be based on the physical characteristics of the subject such as height, weight, and age. In certain embodiments, a sufficient amount of a compound or composition provided herein is administered to achieve a steady-state concentration in blood or serum of the subject of from about 300 to about 4000 ng/mL, from about 400 to about 1600 ng/mL, or from about 600 to about 1200 ng/mL. In some embodiments, loading doses can be administered to achieve steady-state blood or serum concentrations of about 1200 to about 8000 ng/mL, or about 2000 to about 4000 ng/mL for one to five days. In certain embodiments, maintenance doses can be administered to achieve a steady-state concentration in blood or serum of the subject of from about 300 to about 4000 ng/mL, from about 400 to about 1600 ng/mL, or from about 600 to about 1200 ng/mL.

In certain aspects, provided herein are unit dosages comprising a compound, or a pharmaceutically acceptable salt thereof, in a form suitable for administration. Such forms are described in detail herein. In certain embodiments, the unit dosage comprises 1 to 1000 mg, 5 to 250 mg, or 10 to 50 mg active ingredient. In particular embodiments, the unit dosages comprise about 100, 125, 250, 300, 350, 400, 450, or 500 active ingredient. Such unit dosages can be prepared according to techniques familiar to those of skill in the art.

The dosages of the second agents are to be used in the combination therapies provided herein. In certain embodiments, dosages lower than those which have been or are currently being used to prevent or treat HCV infection are used in the combination therapies provided herein. The recommended dosages of second agents can be obtained from the knowledge of those of skill. For those second agents that are approved for clinical use, recommended dosages are described in, for example, Hardman et al., eds., 1996, Goodman & Gilman's The Pharmacological Basis Of Basis Of Therapeutics 9^{th} Ed, Mc-Graw-Hill, New York; Physician's Desk Reference (PDR) 57^{th} Ed., 2003, Medical Economics Co., Inc., Montvale, N.J., which are incorporated herein by reference in its entirety.

In various embodiments, the therapies (e.g., a compound provided herein and the second agent) are administered less than 5 minutes apart, less than 30 minutes apart, 1 hour apart, at about 1 hour apart, at about 1 to about 2 hours apart, at about 2 hours to about 3 hours apart, at about 3 hours to about 4 hours apart, at about 4 hours to about 5 hours apart, at about 5 hours to about 6 hours apart, at about 6 hours to about 7 hours apart, at about 7 hours to about 8 hours apart, at about 8 hours to about 9 hours apart, at about 9 hours to about 10 hours apart, at about 10 hours to about 11 hours apart, at about 11 hours to about 12 hours apart, at about 12 hours to 18 hours apart, 18 hours to 24 hours apart, 24 hours to 36 hours apart, 36 hours to 48 hours apart, 48 hours to 52 hours apart, 52 hours to 60 hours apart, 60 hours to 72 hours apart, 72 hours to 84 hours apart, 84 hours to 96 hours apart, or 96 hours to 120 hours apart. In various embodiments, the therapies are administered no more than 24 hours apart or no more than 48 hours apart. In certain embodiments, two or more therapies are administered within the same patient visit. In other embodiments, the compound provided herein and the second agent are administered concurrently.

In other embodiments, the compound provided herein and the second agent are administered at about 2 to 4 days apart, at about 4 to 6 days apart, at about 1 week part, at about 1 to 2 weeks apart, or more than 2 weeks apart.

In certain embodiments, administration of the same agent may be repeated and the administrations may be separated by at least 1 day, 2 days, 3 days, 5 days, 10 days, 15 days, 30 days, 45 days, 2 months, 75 days, 3 months, or 6 months. In other embodiments, administration of the same agent may be repeated and the administration may be separated by at least at least 1 day, 2 days, 3 days, 5 days, 10 days, 15 days, 30 days, 45 days, 2 months, 75 days, 3 months, or 6 months.

In certain embodiments, a compound provided herein and a second agent are administered to a patient, for example, a mammal, such as a human, in a sequence and within a time interval such that the compound provided herein can act together with the other agent to provide an increased benefit than if they were administered otherwise. For example, the second active agent can be administered at the same time or sequentially in any order at different points in time; however, if not administered at the same time, they should be administered sufficiently close in time so as to provide the desired therapeutic or prophylactic effect. In certain embodiments, the compound provided herein and the second active agent exert their effect at times which overlap. Each second active agent can be administered separately, in any appropriate form and by any suitable route. In other embodiments, the compound provided herein is administered before, concurrently or after administration of the second active agent.

In certain embodiments, the compound provided herein and the second agent are cyclically administered to a patient. Cycling therapy involves the administration of a first agent (e.g., a first prophylactic or therapeutic agents) for a period of time, followed by the administration of a second agent and/or third agent (e.g., a second and/or third prophylactic or therapeutic agents) for a period of time and repeating this sequential administration. Cycling therapy can reduce the development of resistance to one or more of the therapies, avoid or reduce the side effects of one of the therapies, and/or improve the efficacy of the treatment.

In certain embodiments, the compound provided herein and the second active agent are administered in a cycle of less than about 3 weeks, about once every two weeks, about once every 10 days or about once every week. One cycle can comprise the administration of a compound provided herein and the second agent by infusion over about 90 minutes every cycle, about 1 hour every cycle, about 45 minutes every cycle. Each cycle can comprise at least 1 week of rest, at least 2 weeks of rest, at least 3 weeks of rest. The number of cycles administered is from about 1 to about 12 cycles, more typically from about 2 to about 10 cycles, and more typically from about 2 to about 8 cycles.

In other embodiments, courses of treatment are administered concurrently to a patient, i.e., individual doses of the second agent are administered separately yet within a time interval such that the compound provided herein can work together with the second active agent. For example, one component can be administered once per week in combination with the other components that can be administered once every two weeks or once every three weeks. In other words, the dosing regimens are carried out concurrently even if the therapeutics are not administered simultaneously or during the same day.

The second agent can act additively or synergistically with the compound provided herein. In certain embodiments, the compound provided herein is administered concurrently with one or more second agents in the same pharmaceutical composition. In another embodiment, a compound provided herein is administered concurrently with one or more second agents in separate pharmaceutical compositions. In still another embodiment, a compound provided herein is administered prior to or subsequent to administration of a second agent. Also contemplated are administration of a compound provided herein and a second agent by the same or different routes of administration, e.g., oral and parenteral. In certain embodiments, when the compound provided herein is administered concurrently with a second agent that potentially produces adverse side effects including, but not limited to, toxicity, the second active agent can advantageously be administered at a dose that falls below the threshold that the adverse side effect is elicited.

### Kits

Also provided are kits for use in methods of treatment of a liver disorder such as HCV infection. The kits can include a compound or composition provided herein, a second agent or composition, and instructions providing information to a health care provider regarding usage for treating the disorder. Instructions may be provided in printed form or in the form of an electronic medium such as a floppy disc, CD, or DVD, or in the form of a website address where such instructions may be obtained. A unit dose of a compound or composition provided herein, or a second agent or composition, can include a dosage such that when administered to a subject, a therapeutically or prophylactically effective plasma level of the compound or composition can be maintained in the subject for at least 1 days. In some embodiments, a compound or composition can be included as a sterile aqueous pharmaceutical composition or dry powder (e.g., lyophilized) composition.

In some embodiments, suitable packaging is provided. As used herein, “packaging” includes a solid matrix or material customarily used in a system and capable of holding within fixed limits a compound provided herein and/or a second agent suitable for administration to a subject. Such materials include glass and plastic (e.g., polyethylene, polypropylene, and polycarbonate) bottles, vials, paper, plastic, and plastic-foil laminated envelopes and the like. If e-beam sterilization techniques are employed, the packaging should have sufficiently low density to permit sterilization of the contents.

### Methods of Use

Provided herein is a method for inhibiting replication of a virus in a host, which comprises contacting the host with a therapeutically effective amount of Compound I or a pharmaceutically acceptable salt or solvate thereof.

In certain embodiments, provided herein are methods for the treatment and/or prophylaxis of a host infected with Flaviviridae that includes the administration of an effective amount of a solid prodrug form of 2′-chloro-2′-methyl uridine disclosed herein, e.g., Compound I Form I, including a single enantiomer, a mixture of an enantiomeric pair, an individual diastereomer, a mixture of diastereomers, or a tautomeric form thereof; or a pharmaceutically acceptable salt, solvate, prodrug, phosphate, or active metabolite thereof.

In certain embodiments, provided herein are methods for the treatment and/or prophylaxis of a host infected with Flaviviridae that includes the administration of an effective amount of a compound provided herein, or a pharmaceutically acceptable salt thereof. In certain embodiments, provided herein are methods for treating an HCV infection in a subject. In certain embodiments, the methods encompass the step of administering to the subject in need thereof an amount of a compound effective for the treatment or prevention of an HCV infection in combination with a second agent effective for the treatment or prevention of the infection. The compound can be any compound as described herein, and the second agent can be any second agent described in the art or herein. In certain embodiments, the compound is in the form of a pharmaceutical composition or dosage form, as described elsewhere herein.

Flaviviridae are, e.g., discussed generally in Fields Virology, Sixth Ed., Editors: Knipe, D. M., and Howley, P. M., Lippincott Williams & Wilkins Publishers, Philadelphia, Pa., Chapters 25-27, 2013. In a particular embodiment of the invention, the Flaviviridae is HCV. In an alternate embodiment, the Flaviviridae is a flavivirus or pestivirus. In certain embodiments, the Flaviviridae can be from any class of Flaviviridae. In certain embodiments, the Flaviviridae is a mammalian tick-borne virus. In certain embodiments, the Flaviviridae is a seabird tick-borne virus. In certain embodiments, the Flaviviridae is a mosquito-borne virus. In certain embodiments, the Flaviviridae is an Aroa virus. In certain embodiments, the Flaviviridae is a Dengue virus. In certain embodiments, the Flaviviridae is a Japanese encephalitis virus. In certain embodiments, the Flaviviridae is a Kokobera virus. In certain embodiments, the Flaviviridae is a Ntaya virus. In certain embodiments, the Flaviviridae is a Spondweni virus. In certain embodiments, the Flaviviridae is a Yellow fever virus. In certain embodiments, the Flaviviridae is a Entebbe virus. In certain embodiments, the Flaviviridae is a Modoc virus. In certain embodiments, the Flaviviridae is a Rio Bravo virus.

Specific flaviviruses include, without limitation: Absettarov, Aedes, Alfuy, Alkhurma, Apoi, Aroa, Bagaza, Banzi, Bukalasa bat, Bouboui, Bussuquara, Cacipacore, Calbertado, Carey Island, Cell fusing agent, Cowbone Ridge, Culex, Dakar bat, Dengue 1, Dengue 2, Dengue 3, Dengue 4, Edge Hill, Entebbe bat, Gadgets Gully, Hanzalova, Hypr, Ilheus, Israel turkey meningoencephalitis, Japanese encephalitis, Jugra, Jutiapa, Kadam, Kamiti River, Karshi, Kedougou, Kokobera, Koutango, Kumlinge, Kunjin, Kyasanur Forest disease, Langat, Louping ill, Meaban, Modoc, Montana myotis leukoencephalitis, Murray valley encephalitis, Nakiwogo, Naranjal, Negishi, Ntaya, Omsk hemorrhagic fever, Phnom-Penh bat, Powassan, Quang Binh, Rio Bravo, Rocio, Royal Farm, Russian spring-summer encephalitis, Saboya, St. Louis encephalitis, Sal Vieja, San Perlita, Saumarez Reef, Sepik, Sokuluk, Spondweni, Stratford, Tembusu, Tick-borne encephalitis, Turkish sheep encephalitis, Tyuleniy, Uganda S, Usutu, Wesselsbron, West Nile, Yaounde, Yellow fever, Yokose, and Zika.

Pestiviruses are discussed generally in Fields Virology, Sixth Ed., Editors: Knipe, D. M., and Howley, P. M., Lippincott Williams & Wilkins Publishers, Philadelphia, Pa., Chapters 25, 2013. Specific pestiviruses which can be treated include, without limitation: bovine viral diarrhea virus (“BVDV”), classical swine fever virus (“CSFV,” also called hog cholera virus), and border disease virus (“BDV”).

In certain embodiments, the subject can be any subject infected with, or at risk for infection with, HCV. Infection or risk for infection can be determined according to any technique deemed suitable by the practitioner of skill in the art. In certain embodiments, subjects are humans infected with HCV.

In certain embodiments, the subject has never received therapy or prophylaxis for an HCV infection. In further embodiments, the subject has previously received therapy or prophylaxis for an HCV infection. For instance, in certain embodiments, the subject has not responded to an HCV therapy. In certain embodiments, the subject can be a subject that received therapy but continued to suffer from viral infection or one or more symptoms thereof. In certain embodiments, the subject can be a subject that received therapy but failed to achieve a sustained virologic response. In certain embodiments, the subject has received therapy for an HCV infection but has failed to show, for example, a 2 log₁₀ decline in HCV RNA levels after 12 weeks of therapy. It is believed that subjects who have not shown more than 2 log₁₀ reduction in serum HCV RNA after 12 weeks of therapy have a 97-100% chance of not responding.

In certain embodiments, the subject is a subject that discontinued an HCV therapy because of one or more adverse events associated with the therapy. In certain embodiments, the subject is a subject where current therapy is not indicated. For instance, certain therapies for HCV are associated with neuropsychiatric events. Interferon (IFN)-alfa plus ribavirin is associated with a high rate of depression. Depressive symptoms have been linked to a worse outcome in a number of medical disorders. Life-threatening or fatal neuropsychiatric events, including suicide, suicidal and homicidal ideation, depression, relapse of drug addiction/overdose, and aggressive behavior have occurred in subjects with and without a previous psychiatric disorder during HCV therapy. Interferon-induced depression is a limitation for the treatment of chronic hepatitis C, especially for subjects with psychiatric disorders. Psychiatric side effects are common with interferon therapy and responsible for about 10% to 20% of discontinuations of current therapy for HCV infection.

In certain embodiments, provided are methods of treating or preventing HCV infection in subjects where a neuropsychiatric event, such as depression, or risk of such indicates discontinuation of treatment with current HCV therapy. Further provided are methods of treating or preventing HCV infection in subjects where a neuropsychiatric event, such as depression, or risk of such indicates dose reduction of current HCV therapy.

In certain embodiments, the subject has received an HCV therapy and discontinued that therapy prior to administration of a method provided herein. In further embodiments, the subject has received therapy and continues to receive that therapy along with administration of a method provided herein. The methods can be co-administered with other therapy for HBC and/or HCV according to the judgment of one of skill in the art. In certain embodiments, the methods or compositions provided herein can be co-administered with a reduced dose of the other therapy for HBC and/or HCV.

In certain embodiments, provided are methods of treating a subject that is refractory to treatment with interferon. For instance, in some embodiments, the subject can be a subject that has failed to respond to treatment with one or more agents including interferon, interferon α, pegylated interferon α, interferon plus ribavirin, interferon α plus ribavirin and pegylated interferon α plus ribavirin. In some embodiments, the subject can be a subject that has responded poorly to treatment with one or more agents including interferon, interferon α, pegylated interferon α, interferon plus ribavirin, interferon α plus ribavirin and pegylated interferon α plus ribavirin. A pro-drug form of ribavirin, such as taribavirinmay also be used.

In certain embodiments, the subject has, or is at risk for, co-infection of HCV with HIV. For instance, in the United States, 30% of HIV subjects are co-infected with HCV and evidence indicates that people infected with HIV have a much more rapid course of their hepatitis C infection. Maier and Wu, 2002, World J Gastroenterol 8:577-57. The methods provided herein can be used to treat or prevent HCV infection in such subjects. It is believed that elimination of HCV in these subjects will lower mortality due to end-stage liver disease. Indeed, the risk of progressive liver disease is higher in subjects with severe AIDS-defining immunodeficiency than in those without. See, e.g., Lesens et al., 1999, J Infect Dis 179:1254-1258 In certain embodiments, the compounds or compositions are administered to a subject following liver transplant. Hepatitis C is a leading cause of liver transplantation in the U.S., and many subjects that undergo liver transplantation remain HCV positive following transplantation. In certain embodiments, provided are methods of treating such recurrent HCV subjects with a compound or composition provided herein. In certain embodiments, provided are methods of treating a subject before, during or following liver transplant to prevent recurrent HCV infection.

### Assay Methods

Compounds can be assayed for HCV activity according to any assay known to those of skill in the art.

Further, compounds can be assayed for accumulation in liver cells of a subject according to any assay known to those of skill in the art. In certain embodiments, a compound can be administered to the subject, and a liver cell of the subject can be assayed for the compound or a derivative thereof, e.g. a nucleoside, nucleoside phosphate or nucleoside triphosphate derivative thereof.

In certain embodiments, a solid prodrug form of 2′-chloro-2′-methyl uridine is administered to cells, such as liver cells, in vivo or in vitro, and the nucleoside triphosphate levels delivered intracellularly are measured, to indicate delivery of the compound and triphosphorylation in the cell. The levels of intracellular nucleoside triphosphate can be measured using analytical techniques known in the art. Methods of detecting ddATP are described herein below by way of example, but other nucleoside triphosphates can be readily detected using the appropriate controls, calibration samples, and assay techniques.

In certain embodiments, ddATP concentrations are measured in a sample by comparison to calibration standards made from control samples. The ddATP concentrations in a sample can be measured using an analytical method such as HPLC LC MS. In certain embodiments, a test sample is compared to a calibration curve created with known concentrations of ddATP to thereby obtain the concentration of that sample.

In certain embodiments, the samples are manipulated to remove impurities such as salts (Na⁺, K⁺, etc.) before analysis. In certain embodiments, the lower limit of quantitation is about ˜0.2 pmol/mL for hepatocyte cellular extracts particularly where reduced salt is present.

In certain embodiments, the method allows successfully measuring triphosphate nucleotides formed at levels of 1-10,000 pmol per million cells in, e.g., cultured hepatocytes and HepG2 cells.

### EXAMPLES

As used herein, the symbols and conventions used in these processes, schemes and examples, regardless of whether a particular abbreviation is specifically defined, are consistent with those used in the contemporary scientific literature, for example, the Journal of the American Chemical Society or the Journal of Biological Chemistry. Specifically, but without limitation, the following abbreviations may be used in the examples and throughout the specification: g (grams); mg (milligrams); mL (milliliters); μL (microliters); mM (millimolar); μM (micromolar); Hz (Hertz); MHz (megahertz); mmol (millimoles); hr or hrs (hours); min (minutes); MS (mass spectrometry); ESI (electrospray ionization); TLC (thin layer chromatography); HPLC (high pressure liquid chromatography); THF (tetrahydrofuran); CDCl₃ (deuterated chloroform); AcOH (acetic acid); DCM (dichloromethane); DMSO (dimethylsulfoxide); DMSO-d₆ (deuterated dimethylsulfoxide); EtOAc (ethyl acetate); MeOH (methanol); TBME (t-butyl methyl ether); and BOC (t-butyloxycarbonyl).

For all of the following examples, standard work-up and purification methods known to those skilled in the art can be utilized. Unless otherwise indicated, all temperatures are expressed in ° C. (degrees Centigrade). All reactions are conducted at room temperature unless otherwise noted. Synthetic methodologies illustrated herein are intended to exemplify the applicable chemistry through the use of specific examples and are not indicative of the scope of the disclosure.

### Example 1

### Preparation of Compound I Crystalline Form I

Compound I (1.396 kg) and 2-propanol (6.98 L) were charged into vessel 1. The mixture was heated to 75° C. over 62 minutes and held at this temperature for 42 minutes. The mixture was transferred into vessel 2 via a Sartorius Sartopure polish filter (5 μm mesh). The line was rinsed with 2-propanol (2.79 L) pre-heated at 65° C. The mixture was concentrated to final 7.5 liters. Pre-filtered 2-propanol (0.18 L) was charged and the mixture cooled to 53° C. over 40 minutes. Compound I (0.011 kg) was charged. Pre-Filtered n-heptane (3.07 L) was charged over 35 minutes. The mixture was cooled to 20° C. over 90 minutes and held at this temperature for 13 hours. Product was isolated by filtration.

Wet cake was washed with a pre-filtered mixture of 2-propanol (0.98 L) and n-heptane (0.42 L), and deliquored for 30 minutes. Wet cake was dried in a trays-drier under vacuum at 60° C. for 21 hours. A sample was taken for information on residual 2-propanol and n-heptane (NMR analysis). Dry product was offloaded from trays-drier and stored into double plastic bags inside a plastic mousers. Weight of dry product was 1.291 kg (molar yield 0.925%, yield by weight 0.925 wt).

### Solid State ¹³C-NMR for Compound I Crystalline Form I

Form I was characterized based on its solid-state carbon-13 nuclear magnetic resonance (NMR) spectrum. The carbon-13 spectrum was recorded on a Bruker AV500 NMR spectrometer using a Bruker 4 mm H/X/Y BB double resonance CPMAS probe. The spectrum was collected utilizing proton/carbon-13 variable-amplitude cross-polarization (VACP) at 83.3 kHz, with a contact time of 4 ms. Other experimental parameters used for data acquisition were a proton 90-degree pulse of 100 kHz, TPPM decoupling at 100 kHz, a pulse delay of 2.5 s, and signal averaging for 10000 acquisitions. The magic-angle spinning (MAS) rate was set to 13 kHz. A Lorentzian line broadening of 30 Hz was applied to the spectrum before Fourier Transformation. Chemical shifts are reported on the TMS scale using the carbonyl carbon of glycine (176.70 ppm.) as a secondary reference.

### X-Ray Powder Diffraction (XRPD) for Compound I Crystalline Form I

X-Ray Powder Diffraction patterns were collected on a Bruker AXS C2 GADDS diffractometer using Cu Kα radiation (40 kV, 40 mA), automated XYZ stage, laser video microscope for auto-sample positioning and a HiStar 2-dimensional area detector. X-ray optics consists of a single Göbel multilayer mirror coupled with a pinhole collimator of 0.3 mm. A weekly performance check is carried out using a certified standard NIST 1976 Corundum (flat plate).

The beam divergence, i.e. the effective size of the X-ray beam on the sample, was approximately 4 mm. A θ-θ continuous scan mode was employed with a sample—detector distance of 20 cm which gives an effective 2θ range of 3.2°-29.7°. Typically the sample would be exposed to the X-ray beam for 120 seconds. The software used for data collection was GADDS for XP/2000 4.1.43 and the data were analyzed and presented using Diffrac Plus EVA v13.0.0.2 or v15.0.0.0.

Ambient Conditions

Samples run under ambient conditions were prepared as flat plate specimens using powder as received without grinding. Approximately 1-2 mg of the sample was lightly pressed on a glass slide to obtain a flat surface.

Non-Ambient Conditions

Samples run under non-ambient conditions were mounted on a silicon wafer with heat-conducting compound. The sample was then heated to the appropriate temperature at 10° C./min and subsequently held isothermally for 1 minute before data collection was initiated.

X-Ray Powder Diffraction patterns were collected on a Bruker D8 diffractometer using Cu Kα radiation (40 kV, 40 mA), 0-20 goniometer, and divergence of V4 and receiving slits, a Ge monochromator and a Lynxeye detector. The instrument is performance checked using a certified Corundum standard (NIST 1976). The software used for data collection was DiffracPlus XRD Commander v2.6.1 and the data were analyzed and presented using Diffrac Plus EVA v13.0.0.2 or v15.0.0.0.

Samples were run under ambient conditions as flat plate specimens using powder as received. The sample was gently packed into a cavity cut into polished, zero-background (510) silicon wafer. The sample was rotated in its own plane during analysis. The details of the data collection are: Angular range: 2 to 42° 2θ; Step size: 0.05° 2θ; Collection time: 0.5 s/step.

Data were collected on an Oxford Diffraction Supernova Dual Source, Cu at Zero, Atlas CCD diffractometer equipped with an Oxford Cryosystems Cobra cooling device. The data was collected using Cu Kα radiation. Structures were typically solved using either the SHELXS or SHELXD programs and refined with the SHELXL program as part of the Bruker AXS SHELXTL suite (V6.10). Unless otherwise stated, hydrogen atoms attached to carbon were placed geometrically and allowed to refine with a riding isotropic displacement parameter. Hydrogen atoms attached to a heteroatom were located in a difference Fourier synthesis and were allowed to refine freely with an isotropic displacement parameter.

Differential Scanning Calorimetry (DSC) for Compound I Crystalline Form I

DSC data were collected on a TA Instruments Q2000 equipped with a 50 position autosampler. The calibration for thermal capacity was carried out using sapphire and the calibration for energy and temperature was carried out using certified indium. Typically 0.5-3 mg of each sample, in a pin-holed aluminum pan, was heated at 10° C./min from 25° C. to 250° C. A purge of dry nitrogen at 50 mL/min was maintained over the sample.

Modulated temperature DSC was carried out using an underlying heating rate of 2° C./min and temperature modulation parameters of +0.64° C. (amplitude) every 60 seconds (period). The instrument control software was Advantage for Q Series v2.8.0.394 and Thermal Advantage v5.2.6 and the data were analyzed using Universal Analysis v4.7A or v4.4A.

DSC data were collected on a Mettler DSC 823E equipped with a 34 position autosampler. The instrument was calibrated for energy and temperature using certified indium. Typically 0.5-3 mg of each sample, in a pin-holed aluminum pan, was heated at 10° C./min from 25° C. to 250° C. A nitrogen purge at 50 mL/min was maintained over the sample. The instrument control and data analysis software was STARe v9.20.

### Characterization of Compound I Crystalline Form I

The crystalline form of Compound I was characterized using a wide range of techniques to investigate the solid form and chemical properties. The XRPD Diffractogram of Form I is provided in FIG. 1. The table below provides a summary of the characterization data for Compound I crystalline form I.

### Characterization Data Summary

| | |
|---|---|
| Appearance | White powder |
| XRPD | Crystalline, Form I |
| DSC | Sharp endotherm onset 115° C. (−48.2 J/g) - melt, broad |
| | exotherm onset 185° C. (299.4 J/g) - degradation |

The results show that Form I of Compound I is crystalline, chemically pure and made up of very small particles which form agglomerates.

### Polymorphism Studies

Procedures

An extensive range of experiments were carried out using either Compound I crystalline Form I or amorphous Compound I as the input material. A set of 32 solvents with diverse properties (including 3 solvent:water mixtures 95:5 v:v) were selected. All solvents were used for the solubility screen but for the subsequent screens a selection of these solvents were used depending on the solubility.

Procedure 1—Solubility Screen with Crystalline Material

Compound I Form I was weighed into 2 mL vials and treated with increasing amounts of solvent. Solvent was added in 10 or 20 volume portions until the solid dissolved or a maximum volume of 2 mL (80 volumes) had been reached. After each addition of solvent the samples were warmed to 50° C. (except for the samples in diethyl ether, DCM and TBME which were only warmed to 40° C.) and stirred at 400 rpm for a few minutes. Samples that remained suspensions were cooled to room temperature for the addition of more solvent. Samples were left to stir for 1 hour at 50° C. then observations were made. Suspensions were matured (shaken in cycles of 4 hours at R.T./4 hours at 50° C.). After 3 days of maturation samples were equilibrated to room temperature and small amounts of each sample were filtered through glass frits under vacuum and were analyzed by XRPD. Samples that had diffractograms that were new forms or were slightly different from Form I were filtered for further characterization. Samples that remained consistent with Form I were left to mature for an additional 23 days prior to re-analysis by XRPD. At this point samples that remained consistent with Form I were discarded.

Solutions were stirred at 50° C. for 1 hour then were cooled to 5° C. using a linear cooling rate of 0.1° C./min. Samples were stirred at 5° C. for ˜58 hours. Observations were made and any solids were analyzed by XRPD as above and solutions were cooled in the freezer for 3 days. Solids were then analyzed by XRPD and samples that remained solutions were left to evaporate slowly through loosened lids at ambient conditions. When solids were produced they were analyzed by XRPD.

Procedure 2—Maturation Screen with Amorphous Material

All 32 solvents were used for this screen. Compound I (amorphous sample, 25±2 mg) was weighed into 2 mL vials and the samples were warmed to 50° C. (except for the samples in diethyl ether, DCM and TBME which were only warmed to 40° C.). Samples were treated with increasing amounts of solvent at 50° C. until they had dissolved or 80 volumes had been added. Many samples initially dissolved then precipitated back out on stirring. The precipitates were visually different to the amorphous starting material and some formed large crystals. Where dissolution then precipitation occurred no further solvent was added (unless the sample contained only 5 volumes of solvent, in which case an additional 5 volumes was added for ease of filtration).

Samples were left to stir for 1 hour at 50° C., observations were made, and all samples were matured (shaken in cycles of 4 hours at R.T./4 hours at 50° C.). After ˜19.5 hours of maturation samples were equilibrated to room temperature. Samples that contained crystals were examined by optical microscopy. Suitable crystals were submitted for single crystal structure determination. A small amount of each sample that contained solid precipitate was filtered through a glass frit under vacuum and was analyzed by XRPD. Samples that had diffractograms that were new forms or were subtly different to Form I were filtered for further characterization. Samples that were consistent with Form I were discarded. Samples that contained gums were left to mature for a total of 20 days, prior to re-analysis by XRPD. Samples that were solutions were kept for the anti-solvent screen (as cooling and evaporation had already been carried out with the solubility screen samples). The solution in chloroform had not been made during the solubility screen so this sample was cooled in the fridge then the freezer but did not form a precipitate so was left to evaporate at ambient conditions.

Procedure 3—High Temperature Screen with Amorphous Material

Solvents that formed solutions at a concentration of 5 volumes were omitted from this screen. Amorphous Compound I was weighed into 2 mL vials and the samples were warmed to 60° C. (except for the samples in diethyl ether, DCM and TBME which were only warmed to 40° C.). Samples were treated with increasing amounts of solvent at 60° C. to a total of 5, 10, 20, 40, 60, and 80 volumes. Many samples initially dissolved then precipitated back out as solids or gums on stirring. Where dissolution then precipitation of a solid occurred, a total of 10 volumes of solvent was added, where a gum had precipitated up to 80 volumes of solvent was added. Samples were stirred at 60° C. for 1 hour then observations were made.

Samples were left to stir at 60° C. for ˜20 hours, after which samples containing solids were hot filtered through glass frits under vacuum and were analyzed by XRPD. Samples that had diffractograms that were new forms or were slightly different from Form I were filtered for further characterization. Samples that were consistent with Form I were discarded. Samples that were amorphous or contained gums were left to mature for a total of 20 days, prior to reanalysis by XRPD. The sample in 1-propanol contained insufficient solid for XRPD analysis so was left to evaporate. The solution in ethanol was kept for the anti-solvent screen.

Procedure 4—Low Temperature Screen with Amorphous Material

All 32 solvents were used for this screen. Compound I (amorphous sample, 25±2 mg) was weighed into 2 mL vials and the samples were cooled to 0° C. (except for the samples in DMSO, 1,4-dioxane,cyclohexane, and ethylene glycol, which were only cooled to 10° C.). Samples were treated with increasing amounts of solvent at 0° C. to a total of 5, 10, 20, 40, 60, and 80 volumes. Many samples initially dissolved then precipitated back out as solids on stirring. Where dissolution then precipitation of a solid occurred, a total of 10 volumes of solvent was added for ease of filtration. Samples were stirred at 0° C. for 1 hour then observations were made.

Samples were left to stir at 0° C. for ˜23 hours, after which samples containing solids were cold filtered through glass frits under vacuum and were analyzed by XRPD. Samples that had diffractograms that were new forms or were slightly different from Form I were filtered for further characterization. Samples that were consistent with Form I were discarded. Samples that were amorphous were left to stir in the fridge for 19 days, prior to re-analysis by XRPD. Samples that were solutions were kept for the anti-solvent screen.

Procedure 5—High Temperature Screen with Crystalline Material

Less solvent was used in this screen as the aim was to slurry the crystalline material at high temperature to investigate the solid state stability of Form I. Solvents that formed solutions at a concentration of 10 volumes at room temperature in the solubility screen were omitted from this screen. Compound I crystalline form I (25±2 mg) was weighed into 2 mL vials and the samples were warmed to 60° C. (except for the samples in diethyl ether, DCM and TBME which were only warmed to 40° C.). Samples were treated with increasing amounts of solvent at 60° C. to try to form a light suspension (so that there would be some dissolved material to facilitate form conversion but enough solid to analyze). Solvent was added up to a total of 5, 10, 20, 40, 60, and 80 volumes. Samples were stirred at 60° C. for 1 hour then observations were made.

Samples were left to stir at 60° C. for about 21 hours and observations were made. Some samples contained agglomerates of crystals on the walls of the vial, which were analyzed by optical microscopy. None of these samples was suitable for single crystal analysis so all samples containing solids were hot filtered through glass frits under vacuum and were analyzed by XRPD. Samples that had diffractograms that were new forms or were slightly different from Form I were filtered for further characterization. Samples that were consistent with Form I were discarded. Samples that were solutions (or contained insufficient solid for analysis) were cooled to 25° C. over 2 hours. Samples containing solids were analyzed by XRPD and the remaining solution was cooled in the fridge then the freezer overnight and was then left to evaporate at ambient conditions.

Procedure 6—Low Temperature Screen with Crystalline Material

Less solvent was used in this screen as the aim was to slurry the crystalline material at low temperature to assess the solid state stability of Form I. All 32 solvents were used for this screen. Compound I crystalline form I (25±2 mg) was weighed into 2 mL vials and the samples were cooled to 0° C. (except for the samples in DMSO, 1,4-dioxane,cyclohexane and ethylene glycol, which were only cooled to 10° C.). Samples were treated with increasing amounts of solvent at 0° C. to try to form a light suspension (so that there would be some dissolved material to facilitate form conversion but enough solid to analyze). Solvent was added up to a total of 5, 10, 20, 40, 60, and 80 volumes. Samples were stirred at 0° C. for 1 hour then observations were made.

Samples were then left to stir at 0° C. for about 24 hours and observations were made. Samples containing solids were cold filtered through glass frits under vacuum and were analyzed by XRPD. Samples that had diffractograms that were new forms or were slightly different from Form I were filtered for further characterization. Samples that were consistent with Form I were discarded. Samples that were solutions were kept for the anti-solvent screen. The sample in THF contained insufficient solid to analyze so was left to evaporate at ambient conditions prior to XRPD analysis.

Procedure 7—Anti-Solvent Screen with Crystalline Material

All solvents that dissolved the crystalline material at a concentration of 20 volumes were used for this screen. Where possible solutions made in the previous screens were used as the input material for this screen. Where solutions were not available 25 mg (±1 mg) Compound I was weighed into 2 mL vials and the solid was dissolved in the minimum volume of the appropriate solvent. Both heptane and water were used as anti-solvents, where they were miscible with the solvents. Samples were stirred at 25° C. and portions of anti-solvent were added with stirring at 25° C. and observations being made between the addition of portions of anti-solvent. A total of 5, 10, 20, and 40 volumes of anti-solvent was added. Samples were left to stir for 1 hour at 25° C., observations were made, and samples containing solids were filtered through glass frits under vacuum and were analyzed by XRPD. Samples that had diffractograms that were new forms or were slightly different from Form I were filtered for further characterization. Samples that were consistent with Form I were discarded. Gums were matured (shaken in cycles of 4 hours at 50° C./4 hours at RT) for 29 days prior to analysis by XRPD if solids had been formed.

Samples that remained solutions (or cloudy liquids with insufficient solid to analyze) were left to stir at 25° C. for 6 days. After extended stirring several precipitates had formed, which were analyzed by XRPD. Samples that remained solutions were transferred to larger vials and further portions of anti-solvent were added (as above). Up to a total of 160 volumes of antisolvent was added and samples were left to stir at 25° C. overnight, after which all samples contained precipitates, which were analyzed by XRPD.

Results

A representative spectrum is given in FIG. 11. Characteristic carbon-13 chemical shifts and the corresponding relative intensities observed for Form I are given in the table below. Relative peak intensities are normalized to the highest peak. Peak positions and relative intensities were measured with the Bruker TS2.1 software.

| Chemical Shifts [ppm] | Relative Peak Intensity |
|---|---|
| 175.23 | 0.32 |
| 167.26 | 0.25 |
| 151.35 | 0.34 |
| 150.66 | 0.24 |
| 140.95 | 0.21 |
| 129.96 | 0.46 |
| 125.23 | 0.30 |
| 124.09 | 0.34 |
| 103.54 | 0.26 |
| 92.97 | 0.21 |
| 80.46 | 0.23 |
| 77.07 | 0.07 |
| 71.87 | 0.29 |
| 69.07 | 0.26 |
| 62.86 | 0.26 |
| 49.19 | 0.20 |
| 23.78 | 0.35 |
| 21.81 | 1.00 |

Several experiments in the polymorph screen carried out shows Form I of Compound I. An exemplary XRPD pattern is shown in FIG. 1. A partial list of peaks in the XRPD pattern is as follows:

| 2θ angle | Intensity (Arb. Units) |
|---|---|
| 6.8 | >16,000 |
| 7.3 | 1600 |
| 8.2 | 1600 |
| 10.8 | 2850 |
| 13.6 | 1700 |
| 15.7 | 2900 |
| 16.8 | 850 |
| 18.0 | 1900 |
| 18.5 | 3000 |
| 19.3 | 1550 |
| 19.7 | 1100 |
| 21.4 | 1100 |
| 21.8 | 800 |
| 23.6 | 1100 |
| 24.4 | 1050 |
| 24.8 | 900 |
| 26.5 | 1100. |

Two new XRPD patterns were also seen during the screens:

Pattern 2 (Form II): by maturing amorphous material in propyl acetate, by slurrying Form I in IPAc at 60° C. and by slurrying Form I in nitromethane at 0° C.; and

Pattern 3 (Form III): by maturing amorphous material in acetone, by slurrying Form I in cyclohexane at 60° C. and by adding heptane to a solution in 1,4-dioxane.

### Single Crystal Experiments

Two samples were submitted for single crystal X-ray diffraction studies, designated as Solvate 1 (amorphous Compound I in acetone, Form II) and Solvate 2 (amorphous Compound I in 1-propanol, Form III). A single data set was collected for each revealing that they were isostructural with the solvent (acetone or 1-propanol) sitting within voids in the structures.

The absolute stereostructure of Compound I was determined from the acetone structure with a Flack parameter of −0.02(1). This assignment was confirmed by Bayesian statistical analysis on Bijvoet differences which gave the probability of the structure as depicted being correct as 1.000 and the probability it was a racemic twin or false as 0.000. Further details of both structures are discussed below.

Solvate 1 (Form II)

There is one molecule of Compound I and half a molecule of disordered acetone in the asymmetric unit.

### Single Crystal Structure of Solvate 1 (Form II)

| Molecular formula | C_{23·50}H_{30·50}CIN₃O_{9·50}P | | | | |
|---|---|---|---|---|---|
| Molecular weight | 573.43 | | | | |
| Crystal system | Tetragonal | | | | |
| Space group | P4₁2₁2 | a | 15.1295(2) Å, | α | 90°, |
| | | b | 15.1295(2) Å, | β | 90°, |
| | | c | 24.6526(6) Å, | γ | 90° |

| | |
|---|---|
| V | 5643.02(17) Å³ |
| Z | 8 |
| D_{c} | 1.35 g · cm⁻³ |
| μ | 2.22 mm⁻¹ |
| Source, λ | Cu—K(alpha), 1.54178 Å |
| F(000) | 2404 |
| T | 100(2) K |
| Crystal | Colourless equant, 0.3 × 0.2 × 0.2 mm |
| Data truncated to | 0.80 Å |
| θₘₐₓ | 74.45° |
| Completeness | 99.4% |
| Reflections | 16645 |
| Unique reflections | 5745 |
| Rᵢₙₜ | 0.0405 |

Solvate 2 (Form III)

### Single Crystal Structure of Solvate 2 (Form III)

| Molecular formula | C_{22·50}H₂₉CIN₃O_{9·25}P | | | | |
|---|---|---|---|---|---|
| Molecular weight | 555.91 | | | | |
| Crystal system | Tetragonal | | | | |
| Space group | P4(1)2(1)2 | a | 15.1359(3) Å, | α | 90°, |
| | | b | 15.1359(2) Å, | β | 90°, |
| | | c | 24.7096(10) Å, | γ | 90° |

| | |
|---|---|
| V | 5660.9(3) Å³ |
| Z | 8 |
| D_{c} | 1.305 g · cm⁻³ |
| μ | 2.189 mm⁻¹ |
| Source, λ | Cu—K(alpha), 1.54178 Å |
| F(000) | 2328 |
| T | 100(2) K |
| Crystal | Colourless equant, 0.2 × 0.2 × 0.18 mm |
| Data truncated to | 0.80 Å |
| θₘₐₓ | 74.95° |
| Completeness | 98.9% |
| Reflections | 27157 |
| Unique reflections | 5735 |
| Rᵢₙₜ | 0.0626 |

### Amorphous Compound I

Conversion of Form I to Amorphous Material. Compound I crystalline Form I was found to be highly soluble in most primary alcohols. Amorphous material was prepared by fast evaporation from a solution in methanol and also by lyophilisation of a solution in t-butanol. Details are given below.

Methanol Experiments. 101 mg of Compound I crystalline Form I was dissolved in 3 mL methanol and the solvent was removed by rotary evaporation. Sample was dried at ˜25 mbar for 15 mins, forming a gummy material, which was amorphous.

Lyophilisation Experiments. An initial experiment was carried out in 100% t-butanol. 97 mg of Compound I crystalline Form I was dissolved in 12 mL t-butanol, the solution was frozen in cardice/acetone and the sample was lyophilised overnight. The resulting fluffy white solid was amorphous, as measured by XRPD. This sample was characterized fully.

### Characterization of Amorphous Compound I

Full characterization was carried out on a 100 mg scale test batch (lyophilised from 100% t-butanol). Results are given in the table below.

### Characterization of Amorphous Compound I Made by Lyophilisation

| | |
|---|---|
| Appearance | Fluffy white solid |
| XRPD | amorphous |
| ¹H NMR | Consistent with structure. −0.1 mole eq residual tBuOH |
| | (1.4 wt %), some water but no DCM |
| Purity by HPLC | 97.9 area % (Pharmorphix Generic method) |
| Optical microscopy | Dry sample is made up of large agglomerates which disperse |
| | into tiny primary particles in silicon oil. Sample is not |
| | birefringent and particles are long strands or irregular and are |
| | transparent and glassy |
| TGA | 2.6 wt % loss 25-80° C. |
| | 2.2 wt % loss 80-180° C. |
| | Degradation after 180° C. |
| DSC | Small endotherm onset 57° C. (−1.1 J/g), |
| | No melt or re-crystallisation events, |
| | broad exotherm onset 184° C. (255.5 J/g) - degradation |
| DSC at different heating | 50° C./min heating rate: |
| rates | Broad endotherm onset 48° C. (−14.1 J/g), |
| | Broad endotherm onset 124° C. (−14.0 J/g) |
| | Degradation above 200° C. |
| | 2° C./min heating rate: |
| | Shallow endotherm onset 45° C. (−125.1 J/g), |
| | Degradation exotherm onset 165° C. (230.5 J/g) |
| MDSC | Tg inflection point = 63° C. |
| | molecular relaxation endotherm onset 35° C. (−29.8 J/g) |
| Static Stability at | Remains amorphous after 22 days |
| ambient conditions | |
| Static Stability at | Deliquesces to an oil/gum in <1 hour, |
| 40° C./75% RH | remains amorphous after 1 day, |
| | after 15 days amorphous but hint of a peak at 6.5 °20 |
| | after 44 days remains amorphous except for peak at 6.5 °20 |

### Additional Solvate Forms of Compound I

The solvates of Compound I exhibit isomorphism. Based on similarities of the powder X-ray patterns, the solvates can be structurally categorized into two classes: Class I and Class II. Class I solvates showed isomorphism and upon drying the isolated Class I solvate, it can desolvate to Form I. Example of Class I solvates include (but are not limited to) ethyl acetate, acetone, 1-propanol, 2-propanol, and 2-MeTHF solvates. Class II solvates also exhibit isomorphism and desolvation of Class II solvates would also eventually lead to Form I. Example of Class II solvates include (but are not limited to) heptane, xylene, and squalene solvates. In addition to the Class I and Class II solvates, Compound I can crystallize into a hydrate form.

### Preparation and Characterization of Compound I Crystalline Forms IV-VIII

X-Ray Powder Diffraction patterns for Forms IV-VIII and the ¹³C NMR spectrum for Form IV were collected as described above for Form I.

### Form IV (Hydrate Form)

Form I was suspended in 1 mL phosphate buffered solution (50 mM pH 6.8 K phosphate buffer) at room temperature. The mixture was aged for at least ten days yielding a crystalline hydrate form (Form IV). FIG. 6 provides an exemplary XRPD diffractogram of a sample of Form IV of Compound I. Representative peaks are listed below.

| | d- | Relative |
|---|---|---|
| 2-θ, ° | spacing, Å | Intensity, % |
| 5.70 | 15.50 | 100 |
| 9.57 | 9.24 | 18 |
| 10.37 | 8.53 | 6 |
| 11.41 | 7.75 | 43 |
| 13.52 | 6.55 | 49 |
| 13.82 | 6.41 | 8 |
| 14.64 | 6.05 | 65 |
| 15.17 | 5.84 | 4 |
| 17.14 | 5.17 | 11 |
| 17.73 | 5.00 | 13 |
| 18.32 | 4.84 | 6 |
| 19.70 | 4.51 | 4 |
| 20.25 | 4.39 | 15 |
| 20.85 | 4.26 | 36 |
| 21.10 | 4.21 | 36 |
| 21.64 | 4.11 | 26 |
| 22.21 | 4.00 | 6 |
| 22.78 | 3.90 | 7 |
| 23.67 | 3.76 | 4 |
| 24.26 | 3.67 | 4 |
| 24.87 | 3.58 | 11 |
| 25.42 | 3.50 | 2 |
| 26.44 | 3.37 | 4 |
| 27.24 | 3.27 | 14 |
| 27.84 | 3.20 | 9 |
| 28.52 | 3.13 | 7 |
| 29.28 | 3.05 | 5 |
| 29.98 | 2.98 | 4 |
| 30.78 | 2.90 | 3 |
| 31.26 | 2.86 | 2 |
| 32.02 | 2.80 | 2 |
| 32.83 | 2.73 | 3 |
| 33.72 | 2.66 | 2 |
| 34.79 | 2.58 | 1 |
| 36.21 | 2.48 | 1 |
| 37.58 | 2.39 | 2 |
| 39.53 | 2.28 | 2 |
| 40.29 | 2.24 | 2 |

Form IV (hydrate form) was characterized based on its solid-state carbon-13 nuclear magnetic resonance (NMR) spectrum. The carbon-13 spectrum was recorded on a Bruker AV500 NMR spectrometer using a Bruker 4 mm H/X/Y BB double resonance CPMAS probe. The spectrum was collected utilizing proton/carbon-13 variable-amplitude cross-polarization (VACP) at 83.3 kHz, with a contact time of 4 ms. Other experimental parameters used for data acquisition were a proton 90-degree pulse of 100 kHz, TPPM decoupling at 100 kHz, a pulse delay of 2.5 s, and signal averaging for 5520 acquisitions. The magic-angle spinning (MAS) rate was set to 13 kHz. A Lorentzian line broadening of 30 Hz was applied to the spectrum before Fourier Transformation. Chemical shifts are reported on the TMS scale using the carbonyl carbon of glycine (176.70 ppm.) as a secondary reference.

A representative spectrum is given in FIG. 12. Characteristic carbon-13 chemical shifts and the corresponding relative intensities observed for Form IV (Hydrate Form) are given in the table below. Relative peak intensities are normalized to the highest peak. Peak positions and relative intensities were measured with the Bruker TS2.1 software.

| Chemical Shifts | Relative Peak |
|---|---|
| [ppm] | Intensity |
| 175.11 | 0.42 |
| 165.50 | 0.31 |
| 151.48 | 0.61 |
| 141.65 | 0.28 |
| 130.61 | 0.31 |
| 125.82 | 0.38 |
| 123.65 | 0.34 |
| 103.59 | 0.26 |
| 93.24 | 0.33 |
| 81.41 | 0.40 |
| 79.13 | 0.12 |
| 77.44 | 0.11 |
| 74.40 | 0.36 |
| 72.13 | 0.43 |
| 63.52 | 0.38 |
| 50.64 | 0.33 |
| 24.63 | 0.45 |
| 23.34 | 1.00 |
| 22.25 | 0.61 |

### Form V (Heptane Solvate)

Amorphous material was suspended in neat heptane at room temperature. The mixture was aged for at least two weeks yielding a crystalline form (Form V). FIG. 7 provides an exemplary XRPD diffractogram of a sample of Form V of Compound I. Representative peaks are listed below.

| | d- | Relative |
|---|---|---|
| 2-θ, ° | spacing, Å | Intensity, % |
| 6.39 | 13.83 | 100 |
| 6.68 | 13.24 | 84 |
| 8.03 | 11.01 | 22 |
| 8.79 | 10.06 | 9 |
| 10.43 | 8.48 | 28 |
| 11.57 | 7.65 | 22 |
| 12.99 | 6.81 | 14 |
| 13.48 | 6.57 | 26 |
| 14.87 | 5.96 | 30 |
| 15.46 | 5.73 | 43 |
| 16.60 | 5.34 | 21 |
| 17.78 | 4.99 | 67 |
| 18.32 | 4.84 | 35 |
| 18.66 | 4.76 | 30 |
| 18.96 | 4.68 | 57 |
| 19.50 | 4.55 | 19 |
| 20.43 | 4.35 | 28 |
| 20.86 | 4.26 | 36 |
| 22.20 | 4.00 | 17 |
| 23.33 | 3.81 | 38 |
| 23.88 | 3.73 | 16 |
| 24.39 | 3.65 | 23 |
| 24.74 | 3.60 | 26 |
| 25.84 | 3.45 | 13 |
| 26.60 | 3.35 | 21 |
| 27.37 | 3.26 | 7 |
| 29.31 | 3.05 | 12 |
| 29.71 | 3.01 | 8 |
| 30.61 | 2.92 | 6 |
| 34.70 | 2.59 | 2 |

### Form VI (Squalene Solvate)

Form I was suspended in neat squalene at room temperature. The mixture was slurried for at least one week yielding a crystalline form (Form VI). FIG. 8 provides an exemplary XRPD diffractogram of a sample of Form VI of Compound I. Representative peaks are listed below.

| 2-θ, ° | d-spacing, Å | Relative Intensity, % |
|---|---|---|
| 6.36 | 13.89 | 64 |
| 6.63 | 13.34 | 100 |
| 8.07 | 10.96 | 17 |
| 8.58 | 10.30 | 6 |
| 10.32 | 8.57 | 12 |
| 10.65 | 8.31 | 10 |
| 11.13 | 7.95 | 12 |
| 11.63 | 7.61 | 11 |
| 13.05 | 6.78 | 18 |
| 13.40 | 6.61 | 25 |
| 14.81 | 5.98 | 20 |
| 15.22 | 5.82 | 37 |
| 16.25 | 5.45 | 30 |
| 16.66 | 5.32 | 30 |
| 17.53 | 5.06 | 52 |
| 17.84 | 4.97 | 51 |
| 19.02 | 4.67 | 53 |
| 19.61 | 4.53 | 30 |
| 20.23 | 4.39 | 33 |
| 20.87 | 4.26 | 35 |
| 23.46 | 3.79 | 22 |
| 24.62 | 3.62 | 21 |
| 25.59 | 3.48 | 8 |
| 26.34 | 3.38 | 13 |
| 29.21 | 3.06 | 9 |
| 30.53 | 2.93 | 2 |

### Form VII (Xylene Solvate)

Form I was suspended in xylene at 70° C. The mixture was slurried for at least one week yielding a crystalline form (Form VII). FIG. 9 provides an exemplary XRPD diffractogram of a sample of Form VII of Compound I. Representative peaks are listed below.

| 2-θ, ° | d-spacing, Å | Relative Intensity, % |
|---|---|---|
| 6.43 | 13.75 | 100 |
| 6.64 | 13.32 | 83 |
| 8.13 | 10.88 | 11 |
| 10.39 | 8.52 | 6 |
| 11.26 | 7.86 | 9 |
| 13.52 | 6.55 | 25 |
| 15.31 | 5.79 | 33 |
| 16.78 | 5.28 | 22 |
| 17.71 | 5.01 | 32 |
| 18.14 | 4.89 | 34 |
| 19.27 | 4.61 | 27 |
| 21.16 | 4.20 | 19 |
| 23.40 | 3.80 | 20 |
| 24.80 | 3.59 | 18 |
| 26.79 | 3.33 | 13 |
| 29.58 | 3.02 | 7 |

### Form VIII (2-Propanol Solvate)

Form I was suspended in 2-propanol-heptane (2:1, v/v). The mixture was aged for at least one day yielding a crystalline form (Form VIII). FIG. 10 provides an exemplary XRPD diffractogram of a sample of Form VIII of Compound I. Representative peaks are listed below.

| 2-θ, ° | d-spacing, Å | Relative Intensity, % |
|---|---|---|
| 6.77 | 13.06 | 100 |
| 8.17 | 10.82 | 19 |
| 9.05 | 9.77 | 6 |
| 10.76 | 8.22 | 19 |
| 12.01 | 7.37 | 16 |
| 13.39 | 6.61 | 28 |
| 15.60 | 5.68 | 46 |
| 16.19 | 5.48 | 14 |
| 16.70 | 5.31 | 21 |
| 17.79 | 4.99 | 31 |
| 18.31 | 4.85 | 54 |
| 18.64 | 4.76 | 15 |
| 19.06 | 4.66 | 33 |
| 19.61 | 4.53 | 20 |
| 20.30 | 4.38 | 14 |
| 21.12 | 4.21 | 28 |
| 21.54 | 4.13 | 13 |
| 21.79 | 4.08 | 13 |
| 22.04 | 4.03 | 10 |
| 23.41 | 3.80 | 30 |
| 24.15 | 3.69 | 16 |
| 24.71 | 3.60 | 18 |
| 24.92 | 3.57 | 15 |
| 25.19 | 3.53 | 14 |
| 25.45 | 3.50 | 14 |
| 26.21 | 3.40 | 21 |
| 26.81 | 3.33 | 7 |
| 27.36 | 3.26 | 7 |
| 28.07 | 3.18 | 6 |
| 29.08 | 3.07 | 11 |
| 29.92 | 2.99 | 7 |
| 31.07 | 2.88 | 7 |
| 31.49 | 2.84 | 3 |
| 33.15 | 2.70 | 3 |
| 34.22 | 2.62 | 6 |
| 35.84 | 2.51 | 3 |
| 37.75 | 2.38 | 2 |
| 38.55 | 2.34 | 2 |
| 39.50 | 2.28 | 2 |

### Example 2

### Formulations

Pharmaceutical formulations of Compound I (Form I) with samatasvir were produced and tested for stability in 0.01N HCl. In the following formulations, % is (w/w) percentage. Intragranular samatasvir was produced according to U.S. Provisional Patent Application No. 61/948,458, filed 5 Mar. 2014, entitled SOLID FORMS OF A FLAVIVIRIDAE VIRUS INHIBITOR COMPOUND AND SALTS THEREOF, bearing attorney docket no. 11874-295-888 and applicant docket no. IDX 1151, the contents of which are hereby incorporated by reference in their entirety.

### Formulation 1-50 mgA Samatasvir and 50 mg Compound I in 800 mg Tablet

Tablet formulation 1 was produced by combining intragranular samatasvir (25.00%:PVP SDD), silica dioxide (0.54%, colloidal), and magnesium stearate (0.07%) with extragranular Compound I (6.25%), Avicel PH102 (57.39%), copovidone (10.00%), silicon dioxide (0.50%, colloidal), and magnesium stearate (0.25%) and by compressing on a tablet compression machine. The total tablet mass was 800 mg.

The tablet hardness was 22 kP, as measured by hardness tester. Disintegration time in 0.01N HCl was 39 seconds.

### Formulation 2-50 mgA Samatasvir and 100 mg Compound I in 800 mg Tablet

Tablet formulation 2 was produced by combining intragranular samatasvir (25.00%:PVP SDD), silica dioxide (0.54%, colloidal), and magnesium stearate (0.07%) with extragranular Compound I (12.50%), Avicel PH102 (51.14%), copovidone (10.00%), silica dioxide (0.50%, colloidal), and magnesium stearate (0.25%) and by compressing on a tablet compression machine. The total tablet mass was 800 mg.

The tablet hardness was 21 kP, as measured by hardness tester. Disintegration time in 0.01N HCl was 29 seconds.

### Formulation 3-50 mgA Samatasvir and 100 mg Compound I in 800 mg Tablet

Tablet formulation 3 was produced by combining intragranular samatasvir (25.00%:PVP SDD), Compound I (12.50%), silica dioxide (0.54%, colloidal), and magnesium stearate (0.07%) with extragranular Avicel PH102 (49.22%), copovidone (10.00%), silica dioxide (0.50%, colloidal), and magnesium stearate (0.25%) and by compressing on a tablet compression machine. The total tablet mass was 800 mg.

The tablet hardness was 22 kP, as measured by hardness tester. Disintegration time in 0.01N HCl was 2 minutes, 28 seconds.

### Formulation 4-50 mgA Samatasvir and 200 mg Compound I in 800 mg Tablet

Tablet formulation 4 was produced by combining intragranular samatasvir (25.00%:PVP SDD), silica dioxide (0.54%, colloidal), and magnesium stearate (0.07%) with extragranular Compound I (25.00%), Avicel PH102 (38.64%), copovidone (10.00%), silica dioxide (0.50%, colloidal), and magnesium stearate (0.25%) and by compressing on a tablet compression machine. The total tablet mass was 800 mg.

The tablet hardness was 22 kP, as measured by Hardness tester. Disintegration time in 0.01N HCl was 3 minutes and 17 seconds.

### Formulation 5-50 mg Compound I in 200 mg Tablet

Tablet formulation 5 was produced by combining Compound I (50 mg, 25.0%, micronized-seeded), with Avicel PH 105 (10.0%), Avicel PH 102 (15.0%), Pearlitol 200SD (45.0%), Kollidon CL (4.0%) and Ligamed MF-2-V (1.0%) in a compression tablet.

### Formulation 6-150 mg Compound I in 600 mg Tablet

Tablet formulation 6 was produced by combining Compound I (150 mg, 25.0%, micronized-seeded), with Avicel PH 105 (10.0%), Avicel PH 102 (15.0%), Pearlitol 200SD (45.0%), Kollidon CL (4.0%) and Ligamed MF-2-V (1.0%) in a compression tablet.

### Formulation 7-50 mg Compound I in 200 mg Tablet

Tablet formulation 7 was produced by combining Compound I (50 mg, 25.0%, coarser), with Avicel PH 105 (10.0%), Avicel PH 102 (15.0%), Pearlitol 200SD (45.0%), Kollidon CL (4.0%) and Ligamed MF-2-V (1.0%) in a compression tablet.

### Formulation 8-150 mg Compound I in 600 mg Tablet

Tablet formulation 8 was produced by combining Compound I (150 mg, 25.0%, coarser), with Avicel PH 105 (10.0%), Avicel PH 105 (15.0%), Pearlitol 200SD (45.0%), Kollidon CL (4.0%) and Ligamed MF-2-V (1.0%) in a compression tablet.

### Formulation 9-5 mg Compound I in Capsule

Capsule formulation 9 was produced by combining Compound I (5 mg, 33.3%), Pearlitol SD100 (63.7%), and Ac-di-Sol (3%) and by filling the blend into size 0, white, HPMC (hyrdoxypropyl methylcellulose) capsule (Capsugel V-Caps Plus).

### Formulation 10-25 mg Compound I in Capsule

Capsule formulation 10 was produced by combining Compound I (25 mg, 33.3%), Pearlitol SD100 (63.7%), and Ac-di-Sol (3%) and by filling the blend into size 0, white, HPMC (hyrdoxypropyl methylcellulose) capsule (Capsugel V-Caps Plus).

### Formulation 11-50 mg Compound I in Capsule

Capsule formulation 11 was produced by combining Compound I (50 mg, 33.3%), Pearlitol SD100 (63.7%), and Ac-di-Sol (3%) and by filling the blend into size 0, white, HPMC (hyrdoxypropyl methylcellulose) capsule (Capsugel V-Caps Plus).

### Example 3

### Study Design

Part I:

This part was a randomized, double-blind, placebo-controlled, parallel group study in treatment-naïve, Genotype 1 HCV-infected subjects. Thirty (30) subjects were randomized across the treatment arms specified below.

| N¹ | Genotype | Dose | Drug administration |
|---|---|---|---|
| 8:2 | 1 | 50 mg | Compound I or placebo QD × 7 days |
| 8:2 | 1 | 150 mg | Compound I or placebo QD × 7 days |
| 8:2 | 1 | 300 mg | Compound I or placebo QD × 7 days |

¹active:placebo

Based on an interim review of the antiviral activity data, randomization into the 50 mg and 150 mg cohorts was discontinued. Newly randomized subjects were continued in the 300 mg arm until that cohort is filled.

Part II:

This part were a randomized, open-label, parallel group study in treatment-naïve, Genotype 2, 3, 4, 5 and 6 HCV-infected subjects. Thirty (30) subjects were randomized across the treatment arms specified below.

| N | Genotype | Dose | Drug administration |
|---|---|---|---|
| 10 | 2-6 | 50 mg | Compound I QD × 7 days |
| 10 | 2-6 | 150 mg | Compound I QD × 7 days |
| 10 | 2-6 | 300 mg | Compound I QD × 7 days |

Based on an interim review of the antiviral activity data, randomization into the 50 mg and 150 mg cohorts was discontinued. Newly randomized subjects were continued in the 300 mg arm until that cohort was filled.

Dosing, sampling, and analyses for Parts I and II were made according to Tables A and B.

TABLE A

| | STUDY DAY | | | | | | | | | | | | | | | |
|---|---|---|---|---|---|---|---|---|---|---|---|---|---|---|---|---|
| | Screen- | | | | | | | | | | | | | | | 35 |
| | ing −60 | | | | | | | | | | | | | | 13 or | F/U² |
| EVENTS | to −2 | −1 | 1 | 2 | 3 | 4 | 5 | 6 | 7 | 8 | 9 | 10 | 11 | 12 | ET¹ | (±7 days) |
| Informed consent | X | | | | | | | | | | | | | | | |
| Check-in | | X | | | | | | | | | | | | | | |
| Clinic confinement | | X | X | X | X | X | X | X | X | X | X | X | X | X | | |
| Randomization (Groups A, C and D) | Day −14 to 1³ | | | | | | | | | | | | | | | |
| Dosing | | | X | X | X | X | X | X | X | | | | | | | |
| Discharge | | | | | | | | | | | | | | | X | |
| Review Inc/Exc Criteria & Med History | X | X | | | | | | | | | | | | | | |
| Demographics | X | | | | | | | | | | | | | | | |
| Complete Physical Examination | X | | | | | | | | | | | | | | X | X |
| Concomitant Medications | X | X | X | X | X | X | X | X | X | X | X | X | X | X | X | X |
| Assessment of AEs (with symptom-targeted | | | X | X | X | X | X | X | X | X | X | X | X | X | X | X |
| physical exam) | | | | | | | | | | | | | | | | |
| HIV, HBV and HCV⁴ Screen; Hemoglobin | X | | | | | | | | | | | | | | | |
| A⁵ | | | | | | | | | | | | | | | | |
| Drug & Alcohol Screen | X | X | | | | | | | | | | | | | | |
| Vital Signs | X | X | X³ | X | | X | | X | | X | | X | | | X | X |
| Height⁶ and Weight | X | X | X | | | | | | | | | | | | X | X |
| 12-lead ECG⁷ | X | X | X³ | X³ | | X³ | | | | X | | X | | | X | X |
| Hematology, Clinical Chemistry and | X | X | X³ | X³ | X^{3,8} | X³ | X^{3,8} | X³ | | X | | X | | | X | X |
| Coagulation (fasted labs) | | | | | | | | | | | | | | | | |
| GFR | X | X | X³ | X³ | | X³ | | X³ | | X | | X | | | X | X |
| Pregnancy Test for Females⁹ | X | X | | | | | | | | | | | | | X | X |
| FSH¹⁰ | X | | | | | | | | | | | | | | | |
| Urinalysis | X | X | X³ | X³ | | X³ | | X³ | | X | | X | | | X | X |
| DNA samples for IL28B (Groups C, D and | | | X³ | | | | | | | | | | | | | |
| E) | | | | | | | | | | | | | | | | |
| Plasma PK Sample Collection | | | X | X | X | X | X | X | X | X | X | X | X | X | | |
| HCV Genotyping (Groups C, D and E) | X | | | | | | | | | | | | | | | |
| HCV RNA Quantitation (Groups C, D and | X | X | X | X | X | X | X | X | X | X | X | X | X | X | X | X |
| E) | | | | | | | | | | | | | | | | |
| Plasma and/or Serum Samples Stored for | X | X | X | X | X | X | X | X | X | X | X | X | X | X | X | X |
| Adjunctive Testing Retesting¹² | | | | | | | | | | | | | | | | |

¹Early Termination.
²Follow-up (26 days after last study drug administration).
³Predose.
⁴HCV screen done in healthy volunteers only.
⁵HCV-infected subjects only.
⁶Height will be measured at screening only.
⁷Three consecutive determinations collected within 5 minutes apart from each other to be performed at each scheduled timepoint. ECGs taken after Day 1 should be time-matched to the Day 1 (predosage) ECG.
⁸ALT and AST only.
⁹Serum pregnancy test at Screening urine of serum pregnancy tests at other timepoints.
¹⁰Females of non-childbearing potential only.
¹²Stored samples may be used for retesting clinical laboratory parameters. In addition, stored samples will be dedicated for HCV antiviral resistance testing (HCV-infected subjects only). Samples collected may also be stored for retesting.

TABLE B
Plasma PK samples (hours post previous dose)

```
Screening and Day −1
  HCV RNA sample only* (no PK)
Day 1
  Prior to dosing*
  0.5, 1, 2*, 3, 4*, 6, 8*, 12*, 16*, 20* h post Day 1 dose
Day 2
  Prior to dosing (24 h post Day 1 dose)*, 12 h (36 h post Day 1 dose)*
Day 3
  Prior to dosing (48 h post Day 1 dose)*
Day 4
  Prior to dosing (72 h post Day 1 dose)*
Day 5-6
  Prior to dosing (96 h and 120 h post Day 1 dose)*
Day 7
  Prior to dosing*
  0.5, 1, 2, 3, 4, 6, 8, 12, 16, 20 h post Day 7dose
Day 8
  24 h*, 36 h post Day 7 dose
Day 9
  48 h post Day 7 dose*
Day 10
  72 h post Day 7 dose*
Day 11
  96 h post Day 7 dose*
Day 12
  120 h post Day 7 dose*
Day 13
  HCV RNA sample only* (no PK)
Day 35
  HCV RNA sample only* (no PK)
*Indicates sample is also taken for HCV RNA quantitation at this timepoint for Groups C, D and Cohort 2e only. Time of PK sampling and dosing must be recorded. For plasma PK samples, all predose timepoints must be drawn within 30 minutes prior to dosing. For post dose timepoints, a ±5 min window for timepoints ≦4 h, ±10 min for timepoints between 4 and 24 hr; and ±30 min for all other post dose timepoints will be allowed.
```

To participate in the study, subjects must meet all of the following inclusion criteria:

1. Read and signed the written informed consent form (ICF) after the nature of the study has been fully explained.

2. Male or female subjects between 18 and 55 years of age, inclusive (or the legal age of consent per local regulations).

3. Minimum body weight of 50 kg and body mass index (BMI) of 18-32 kg/m2 (Group A in Example 4) or 18-35 kg/m2 (Groups B-E), inclusive.

4. All subjects of childbearing potential must have agreed to use a double-method of birth control (one of which must be a barrier) from Screening through at least 90 days after the last dose of the study drug.

Non-childbearing potential is defined as: Females: postmenopausal, defined as amenorrheic for at least 2 years and serum follicle stimulating hormone (FSH) level consistent with postmenopausal status at Screening, or a self-reported hysterectomy, bilateral oophorectomy or bilateral tubal ligation at least 6 months prior to Screening; and Males: a self-reported vasectomy at least 6 months prior to Screening.

5. Females must have a negative serum beta-human chorionic gonadotropin (β-HCG) at Screening and a negative pregnancy test (urine or β-HCG) at Day −1.

6. Male subjects must have agreed not to donate sperm from the first dose through 90 days after the last dose of study drug.

7. Subject must not have consumed grapefruit or grapefruit juice within 7 days of reporting to the clinic on Day −1, and agrees not to do so through the end of the study.

8. Subject is, in the opinion of the investigator, willing and able to comply with the study drug regimen and all other study requirements.

Subjects in Parts I and II must be HCV treatment-naïve (i.e., no prior exposure to any antiviral treatment for hepatitis C infection).

Subjects will be excluded from the study if they meet any of the following exclusion criteria: (1) Female subject is pregnant or breastfeeding; (2) Co-infected with hepatitis B virus (HBV, hepatitis B surface antigen (HBsAg) positive) and/or human immunodeficiency virus (HIV); (3) Donated more than 500 mL of blood or had significant blood loss 60 days prior to dosing; (4) Abuse of alcohol and/or drugs that could interfere with adherence to study requirements as judged by the investigator; (5) Positive screen result for drugs of abuse or alcohol at Screening or on Day −1 (Note: entry of HCV-infected subjects with a positive THC result is at the discretion of the investigator); (6) Concomitant use of any known major inhibitor or inducer of CYP 3A4 (A washout period of at least 5 half-lives of the CYP 3A4 drug must be observed prior to study drug dosing, if the investigator feels that the drug can be safely discontinued or substituted for the duration of the study); (7) Concomitant use of herbal or dietary supplements (A washout period of at least 7 days must be observed prior to study drug dosing); (8) Concomitant use of acid blockers (e.g., proton-pump inhibitors) (A washout period of at least 5 half-lives must be observed prior to study drug dosing, if the investigator feels that the drug can be safely discontinued or substituted for the duration of the study. Antacids (stomach acid neutralizers such as calcium carbonate or aluminum hydroxide based products) will be allowed during the study, except ±4 h of dosing); (9) Concomitant use of the following medications: amlodipine, aripiprazole, atorvastatin, carvedilol, diazepam, diltiazem, fluoxetine, isradipine, perphenazine, procainamide, simvastatin, thioridazine or verapamil (A washout period of at least 5 half-lives must be observed prior to initiating study drug dosing, if the investigator feels that the drug can be safely discontinued or substituted for the duration of the study); (10) Use of other investigational drugs within 60 days of dosing, or plans to enroll in another clinical trial of an investigational agent while participating in the present study; (11) Subject with intestinal malabsorption (e.g., structural defects, digestive failure or enzyme deficiencies with the exception of lactose intolerance); (12) Subject with known allergy to the study medication or any of its components; (13) Cardiac disease, family history of congenital heart disease, family history of prolonged QT, or family history of sudden death of unknown etiology; (14) One or more of the following ECG abnormalities: First degree, or greater, heart block; QTcF interval≧430 ms for males or ≧450 ms for females, at Screening, Day −1, and Day 1 (prior to the first dose) (Note: The average of the triplicate at each timepoint should be used to assess the QTcF); and Clinically significant abnormal ECG at Screening or Day −1, as determined by the investigator; (15) At Screening or Day −1: an estimated glomerular filtration rate (GFR)<60 mL/min/1.73 m2 as estimated by the Modification of Diet in Renal Disease (MDRD) formula; (16) Hemoglobin (Hgb)<13.0 g/dL for males, <12.0 g/dL for females; (17) Any clinically significant medical condition that, in the opinion of the investigator, would jeopardize the safety of the subject or impact the validity of the study results; (18) Clinical (in the opinion of the investigator) or laboratory evidence of cirrhosis (e.g., Metavir 4 or Ishak 6) (Note: A prior liver biopsy is not required for inclusion into this short-term study); (19) History or signs of decompensated liver disease: ascites, variceal bleeding, hepatic encephalopathy, spontaneous bacterial peritonitis, or other clinical signs of portal hypertension or hepatic insufficiency; (20) History of hepatocellular carcinoma (HCC) or findings suggestive of possible HCC; (21) Active clinically significant diseases including: Primary or secondary causes of liver disease (other than hepatitis C) (For example, alcoholism, autoimmune hepatitis, nonalcoholic steatohepatitis, drug-related liver disease, malignancy with hepatic involvement, hemochromatosis, alpha-1 antitrypsin); deficiency, Wilson's Disease, Dubin-Johnson syndrome, other congenital or metabolic conditions affecting the liver, etc.; Malignant disease or suspicion or history of malignant disease within previous 5 years (except for adequately treated basal cell carcinoma); and Poorly controlled diabetes mellitus as evidenced by hemoglobin Alc≧8.5% at Screening; (22) Requires frequent or prolonged use of systemic corticosteroids or other immunosuppressive drugs (e.g., for organ transplantation or autoimmune conditions) (Topical or inhaled corticosteroids are permitted); (23) Abnormal values at Screening or Day −1: ALT or AST>5× upper limit of normal (ULN); platelet count<120×109/L; and Any other laboratory abnormality that is considered to be clinically significant by the investigator(s).

Subjects with test results which do not meet the above inclusion/exclusion criteria may have the underlying test repeated once if it is thought to represent a laboratory error, a reversible, clinically insignificant intermittent condition, or is not consistent with the subject's historical values. If inclusion/exclusion criteria are not met after the repeat test, the subject should be considered a screen failure and should not be enrolled in the study. Re-screening of prior screen failures is not permitted; however, subjects who were previously screened and eligible, but could not be enrolled into a cohort within the screening window may be re-screened (in this case, the subject's prior HCV genotype or HCV RNA quantitation does not need to be repeated).

Subjects may voluntarily withdraw from the study, or be removed from the study at the discretion of the investigator or Sponsor at any time. The investigator may withdraw a subject at any time if it is determined that continuing the study would result in a significant safety risk to the subject.

At Screening, each subject will be assigned a unique subject number. If a subject fails to be randomized, the reason for not being randomized should be documented in the site's source documents.

Compound I was provided as size 0, white, hard hydroxypropyl methylcellulose (HPMC) capsules for oral administration at 5 mg, 25 mg and 50 mg strengths. The same powder blend containing 33.3% weight/weight (w/w) Compound I, a diluent, and a disintegrant is used to fill the capsules for all dose strengths. Matching placebo capsules will also be provided. Storage conditions will be provided on the study drug container label.

For Part I, subjects were randomized via a computer-generated randomization list to receive either active study drug or placebo. For Part II, subjects were randomized equally across three treatment groups to receive open-label treatment.

For Part I, Treatment assignments were blinded to the Investigator(s) and subjects for the entire study (although a designated site pharmacist(s) (or designee) will remain unblinded in order to dispense study drug or placebo). For the safety/PK reviews, the Sponsor and Principal Investigator(s) (or designee) will review the data to make a decision about dose escalation or advancement to the next part of the protocol.

Dosing occurred between approximately 08:00 AM and 10:00 AM on each dosing day. Water was permitted ad libitum except for 1 hour before and after dosing. Dosing was immediately followed by administration of 240 mL (8 oz.) of water. Meals and beverages served within 24 hours prior to and post dosing were xanthine-free and caffeine-free. Grapefruit and grapefruit juice were not allowed within 7 days before reporting to the clinic on Day −1 and through the end of the study.

Dose adjustments and interruptions were not permitted in this study. If a subject was experiencing an AE that requires dose adjustment and/or interruptions, the study drug would be permanently discontinued.

Concomitant medications were administered only as medically necessary during the study. Any concomitant medications would be initiated with caution, since they may increase the possibility of AEs. All concomitant medications were recorded in the site's study records and entered into the appropriate case report form (CRF) page.

Use of the following medications was prohibited during the study as follows: all investigational drugs other than Compound I; all other treatments for HCV; pharmaceutical agents associated with significant renal or hepatic toxicity; drugs which are major inhibitors or inducers of CYP 3A; concomitant use of acid blockers; antacids (stomach acid neutralizers such as calcium carbonate or aluminum hydroxide-based products) were allowed during the study, except ±4 h of dosing; concomitant use of prescription medications (with the exception of hormonal contraceptives) or systemic over-the-counter medications (Group A in Example 4 only); all herbal medications (e.g., St. John's Wort, echinacea, gingko, etc.) and dietary supplements; and concomitant use of the following medications: amlodipine, aripiprazole, atorvastatin, carvedilol, diazepam, diltiazem, fluoxetine, isradipine, perphenazine, procainamide, simvastatin, thioridazine or verapamil.

### Results

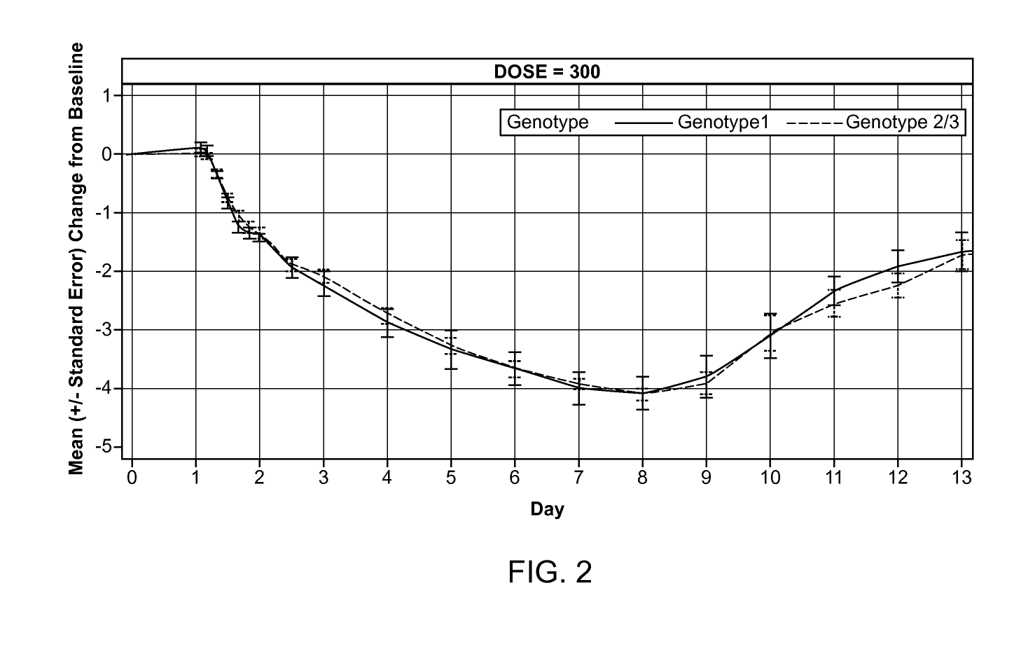

Compound I was well-tolerated with no observed patterns of clinical or laboratory abnormalities. Compound I demonstrated potent pan-genotypic activity in a dose-dependent manner. FIG. 2 shows the reduction in maximal viral load as measured for subjects with Genotype 1 and 2/3 after the subjects were treated with Compound I in the amount of 300 mg/day for 7 days, In the 8 genotype 1 HCV-infected patients receiving the 300 mg dose of Compound I, the mean maximal viral load reduction was 4.2 logs. The response was similar between patients with genotype 1a and genotype 1b HCV infection. In the 10 genotype 2 or 3 HCV-infected patients receiving the 300 mg dose, the mean maximal viral load reduction was 4.3 logs. The response was also similar between patients with genotype 2 and genotype 3 HCV infection.

### Example 4

### Study Design

Six sequential cohorts of 8 health volunteers per cohort were randomized via a computer-generated randomization list in the ratio of 2:6. Two of the healthy volunteers (HV) received placebo and the remaining six received Compound I under fasted conditions at single ascending doses of 10, 25, 50, 150, or 300 mg, or multiple doses at 300 mg QD for 7 days (below, “Group A”).

| Cohort | N¹ | Dose | Drug administration |
|---|---|---|---|
| 1a | 6:2 | 10 mg | Compound I or placebo × 1 day |
| 2a | 6:2 | 25 mg | Compound I or placebo × 1 day |
| 3a | 6:2 | 50 mg | Compound I or placebo × 1 day |
| 4a | 6:2 | 150 mg | Compound I or placebo on days 1 and 7 |
| 5a | 6:2 | 300 mg | Compound I or placebo × 1 day |
| 6a | 6:2 | 300 mg | Compound I or placebo QD × 7 days |

¹active:placebo

In addition to the inclusion and exclusion criteria set forth in Example 3, subjects in this study must not have smoked or used nicotine-containing products within 6 months prior to Day −1 and agreed not to do so for the duration of the study.

The baseline characteristics of healthy volunteers are shown in the table below.

| | Placebo | 10 mg | 25 mg | 50 mg | 150 mg | 300 mg | 300 mg QD | All |
|---|---|---|---|---|---|---|---|---|
| Demographics | (n = 12) | (n = 6) | (n = 6) | (n = 6) | (n = 6) | (n = 6) | (n = 6) | (N = 48) |
| Age (yr) | 35.4 ± 7.04 | 29.2 ± 7.36 | 33.3 ± 15.1 | 31.3 ± 11.6 | 35.5 ± 11.8 | 35.2 ± 9.87 | 35.3 ± 12.7 | 33.8 ± 10.2 |
| Weight (kg) | 70.6 ± 10.3 | 65.0 ± 10.4 | 71.8 ± 13.3 | 74.1 ± 13.4 | 66.6 ± 4.76 | 63.6 ± 7.24 | 67.7 ± 4.71 | 68.8 ± 9.81 |
| BMI (kg/m²) | 24.5 ± 2.22 | 23.0 ± 2.56 | 24.9 ± 2.78 | 25.8 ± 4.06 | 25.3 ± 3.27 | 24.3 ± 2.13 | 24.2 ± 1.81 | 24.5 ± 2.65 |
| Gender (n, % | 8 (66.7) | 2 (33.3) | 4 (66.7) | 3 (50.0) | 1 (16.7) | 2 (33.3) | 2 (33.3) | 22 (45.8) |
| male) | | | | | | | | |
| Race (n, %) | | | | | | | | |
| White | 8 (66.7) | 5 (83.3) | 5 (83.3) | 5 (83.3) | 5 (83.3) | 5 (83.3) | 5 (83.3) | 38 (79.2) |
| Black or | 2 (16.7) | 1 (16.7) | 1 (16.7) | 1 (16.7) | 1 (16.7) | 0 | 1 (16.7) | 7 (14.6) |
| African | | | | | | | | |
| American | | | | | | | | |
| Other | 2 (16.7) | 0 | 0 | 0 | 0 | 1 (16.7) | 0 | 3 (6.3) |

Dosing in genotype 1 HCV-infected subjects naïve to direct-acting HCV antiviral agents was initiated after the safety and pharmacokinetic data from the corresponding dose level in the healthy subjects were reviewed.

Five sequential cohorts of 3 HCV-infected subjects (HCV) per cohort received under fasted conditions open-label Compound I at single ascending doses of 10, 25, 50, 150, and 300 mg (below, “Group B”).

| Cohort | Genotype | N | Dose | Drug administration |
|---|---|---|---|---|
| 1b | 1 | 3 | 10 mg | Compound I × 1 day |
| 2b | 1 | 3 | 25 mg | Compound I × 1 day |
| 3b | 1 | 3 | 50 mg | Compound I × 1 day |
| 4b | 1 | 3 | 150 mg | Compound I × 1 day |
| 5b | 1 | 3 | 300 mg | Compound I × 1 day |

In addition to the inclusion and exclusion criteria set forth in Example 3, subjects in Group may have received prior treatment with interferon (IFN) and rivavirin for hepatitis C infection but not treatment with any direct-acting antivirals. Patients with (a) clinical or laboratory evidence of cirrhosis, (b) history or signs of decompensated liver diseases (ascites, variceal bleeding, hepatic encephalophathy, spontaneous bacterial peritonitis) or other clinical signs of portal hypertension or hepatic insufficiency, (c) history of hepatocellular carcinoma or findings suggestive of HCC, (d) active clinically significantly diseases (primary or secondary causes of liver disease (other than hepatitis C), for example, alcoholism, autoimmune hepatitis, nonalcoholic steatohepatitis, drug-related liver disease, malignancy with hepatic involvement, hemochromatosis, alpha-1 antitrypsin deficiency, Wilson's Disease, Dubin-Johnson syndrome, other congenital or metabolic conditions affecting the liver, etc.; malignant disease or suspicion or history of malignant disease within previous 5 years (except for adequately treated basal cell carcinoma); or poorly controlled diabetes mellitus as evidenced by hemoglobin A1c≧8.5% at Screening), (d) requires frequent or prolonged use of systemic corticosteroids or other immunosuppressive drugs; or abnormal values of ALT (greater than 5× upper limit or normal), AST (greater than 5× upper limit or normal), platelet count (less than 120×10⁹/L), or any other laboratory abnormality at screening or Day −1 were excluded.

The baseline characteristics of HCV-infected subjects are shown below.

| | 10 mg | 25 mg | 50 mg | 150 mg | 300 mg | All |
|---|---|---|---|---|---|---|
| Demographics | (n = 3) | (n = 3) | (n = 3) | (n = 3) | (n = 3) | (N = 15) |
| Age (yr) | 37.0 ± 9.54 | 42.3 ± 7.23 | 46.7 ± 4.16 | 43.3 ± 5.69 | 41.3 ± 8.96 | 42.1 ± 7.04 |
| Weight (kg) | 84.8 ± 20.3 | 74.9 ± 3.05 | 72.4 ± 5.02 | 66.4 ± 17.0 | 83.1 ± 17.6 | 76.3 ± 14.1 |
| BMI (kg/m2) | 26.7 ± 6.36 | 24.7 ± 1.88 | 26.1 ± 3.00 | 21.6 ± 2.42 | 27.3 ± 8.03 | 25.3 ± 4.70 |
| Gender (n, % | 3 (100) | 3 (100) | 2 (66.7) | 1 (33.3) | 2 (66.7) | 11 (73.3) |
| male) | | | | | | |
| Race (n, %) | | | | | | |
| White | 3 (100) | 3 (100) | 3 (100) | 2 (66.7) | 3 (100) | 14 (93.3) |
| American | 0 | 0 | 0 | 1 (33.3) | 0 | 1 (6.7) |
| Indian or | | | | | | |
| Alaska Native | | | | | | |

The dosing designs for Groups A and B studies are shown below.

| Period | 1 | 2 | 3 | 4 | 5 | 6 |
|---|---|---|---|---|---|---|
| HV | 10 mg | | | | | |
| | | 25 mg | | | | |
| | | | 50 mg | | | |
| HCV | | 10 mg | | 150 mg | | |
| | | | 25 mg | | 300 mg | |
| | | | | 50 mg | | 300 mg |
| | | | | | | QD × 7 d |
| | | | | | 150 mg | |
| | | | | | | 300 mg |

Compound I was provided as size 0, white, hard hydroxypropyl methylcellulose (HPMC) capsules for oral administration at 5 mg, 25 mg, and 50 mg strengths. The same powder blend containing 33.3% weight/weight (w/w) Compound I, a diluent, and a disintegrant is used to fill the capsules for all dose strengths. Matching placebo capsules will also be provided. Storage conditions will be provided on the study drug container label.

For Groups A and B, dosing occurred between approximately 08:00 AM and 10:00 AM on each dosing day. Dosing was fasted for all subjects in Groups A and B. For fasted dosing, subjects observed a fasting period of approximately 10 hours prior to each dose and for an additional 4 hours post-dose.

### Pharmacokinetic and Safety Analysis

Plasma samples were obtained up to 120 hours after dosing and the levels of Compound I and its Nucleoside metabolite were measured by using validated LC/MS/MS methodology. Plasma pharmacokinetic parameters were obtained by using non-compartmental analysis with parameters reported as mean±standard deviation (SD) except for Tₘₐₓ where median (min-max) is shown.

Safety measurements included clinical history, routine laboratory evaluation, physical examination, 12-lead ECG, vital signs, adverse event (AE) assessment.

### Results

Safety summary in health volunteers is shown below.

| | Placebo | 10 mg | 25 mg | 50 mg | 150 mg | 300 mg | 300 mg QD |
|---|---|---|---|---|---|---|---|
| Adverse event¹ | (n = 12) | (n = 6) | (n = 6) | (n = 6) | (n = 6) | (n = 6) | (n = 6) |
| Subjects with AE (n, %) | 6 (50.0) | 0 | 2 (33.3) | 2 (33.3) | 4 (66.7) | 3 (50.0) | 2 (33.3) |
| Dizziness | 1 (8.3) | 0 | 0 | 1 (16.7) | 2 (33.3) | 1 (16.7) | 0 |
| Headache | 2 (16.7) | 0 | 0 | 1 (16.7) | 1 (16.7) | 1 (16.7) | 0 |
| Nausea | 0 | 0 | 0 | 0 | 2 33.3) | 1 (16.7) | 0 |
| Abdominal discomfort | 0 | 0 | 0 | 1 (16.7) | 1 (16.7) | 0 | 0 |
| Abnormal feces | 0 | 0 | 0 | 0 | 2 (33.3) | 0 | 0 |
| Dry mouth | 1 (8.3) | 0 | 0 | 0 | 1 (16.7) | 0 | 0 |
| Vessel puncture | 0 | 0 | 0 | 0 | 0 | 1 (16.7) | 1 (16.7) |
| site bruise | | | | | | | |
| Vessel puncture | 2 (16.7) | 0 | 0 | 0 | 0 | 0 | 0 |
| site pain | | | | | | | |
| Constipation | 1 (8.3) | 0 | 0 | 0 | 0 | 0 | 0 |
| Dry skin | 1 (8.3) | 0 | 0 | 0 | 0 | 0 | 0 |
| Erythema | 0 | 0 | 0 | 0 | 1 (16.7) | 0 | 0 |
| Flushing | 0 | 0 | 0 | 0 | 1 (16.7) | 0 | 0 |
| Menstruation delayed | 0 | 0 | 0 | 0 | 0 | 0 | 1 (16.7) |
| Musculoskeletal | 0 | 0 | 0 | 0 | 0 | 1 (16.7) | 0 |
| stiffness | | | | | | | |
| Palpitations | 0 | 0 | 0 | 0 | 0 | 0 | 1 (16.7) |
| Pancreatic enzymes | 0 | 0 | 1 (16.7) | 0 | 0 | 0 | 0 |
| increased | | | | | | | |
| Procedural nausea | 0 | 0 | 1 (16.7) | 0 | 0 | 0 | 0 |
| Vomiting | 0 | 0 | 0 | 0 | 1 (16.7) | 0 | 0 |

¹Classified according to MedDRA V15.1

Safety summary in health volunteers is shown below.

| | 10 mg | 25 mg | 50 mg | 150 mg | 300 mg |
|---|---|---|---|---|---|
| Adverse events¹ | (n = 3) | (n = 3) | (n = 3) | (n = 3) | (n = 3) |
| Subjects with AE (n, %) | 1 (33.3) | 2 (66.7) | 1 (33.3) | 2 (66.7) | 2 (66.7) |
| Fatigue | 0 | 1 (33.3) | 0 | 1 (33.3) | 0 |
| Constipation | 0 | 1 (33.3) | 0 | 0 | 0 |
| Diarrhea | 0 | 0 | 0 | 0 | 1 (33.3) |
| Dry mouth | 0 | 0 | 0 | 1 (33.3) | 0 |
| Dry skin | 0 | 0 | 0 | 1 (33.3) | 0 |
| Dysuria | 0 | 0 | 1 (33.3) | 0 | 0 |
| Feces hard | 0 | 0 | 0 | 0 | 1 (33.3) |
| Flatulence | 0 | 1 (33.3) | 0 | 0 | 0 |
| Headache | 0 | 1 (33.3) | 0 | 0 | 0 |
| Myalgia | 1 (33.3) | 0 | 0 | 0 | 0 |
| Neck pain | 0 | 1 (33.3) | 0 | 0 | 0 |
| Pruritus | 0 | 0 | 0 | 1 (33.3) | 0 |
| Pruritus generalized | 0 | 0 | 0 | 1 (33.3) | 0 |
| Vessel puncture site | 0 | 0 | 0 | 0 | 1 (33.3) |
| bruise | | | | | |

¹Classified according to MedDRA V15.1

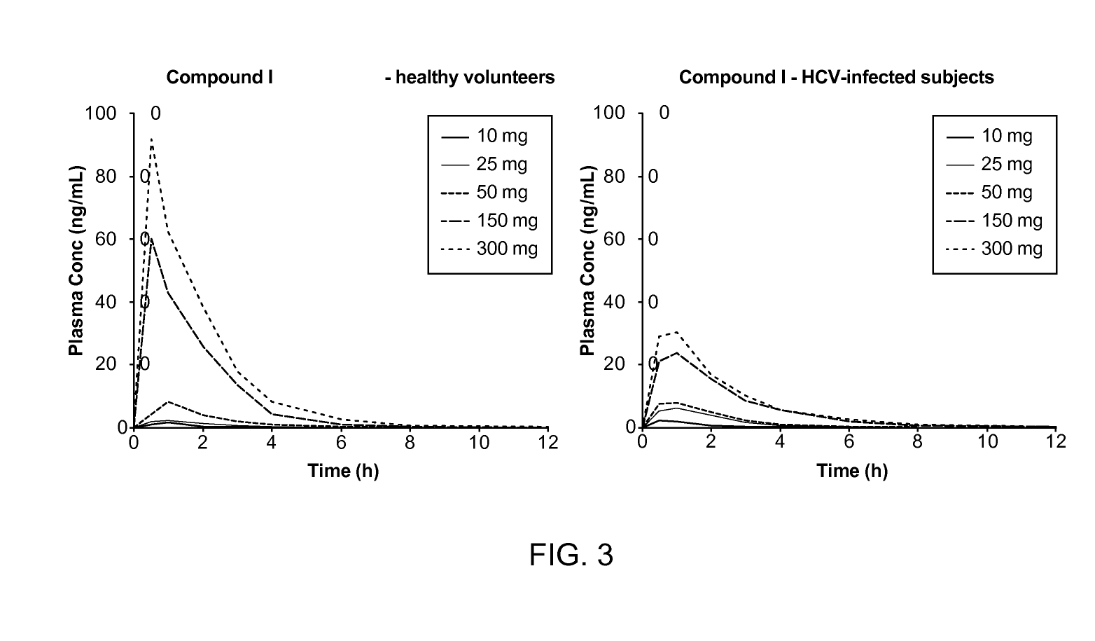

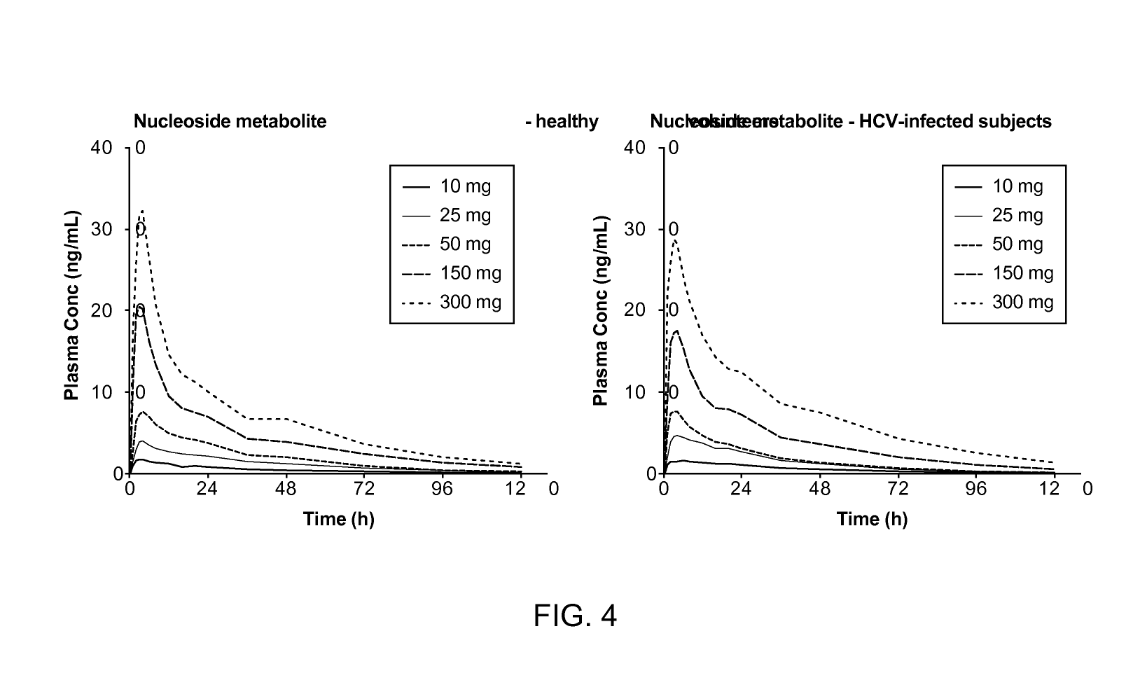

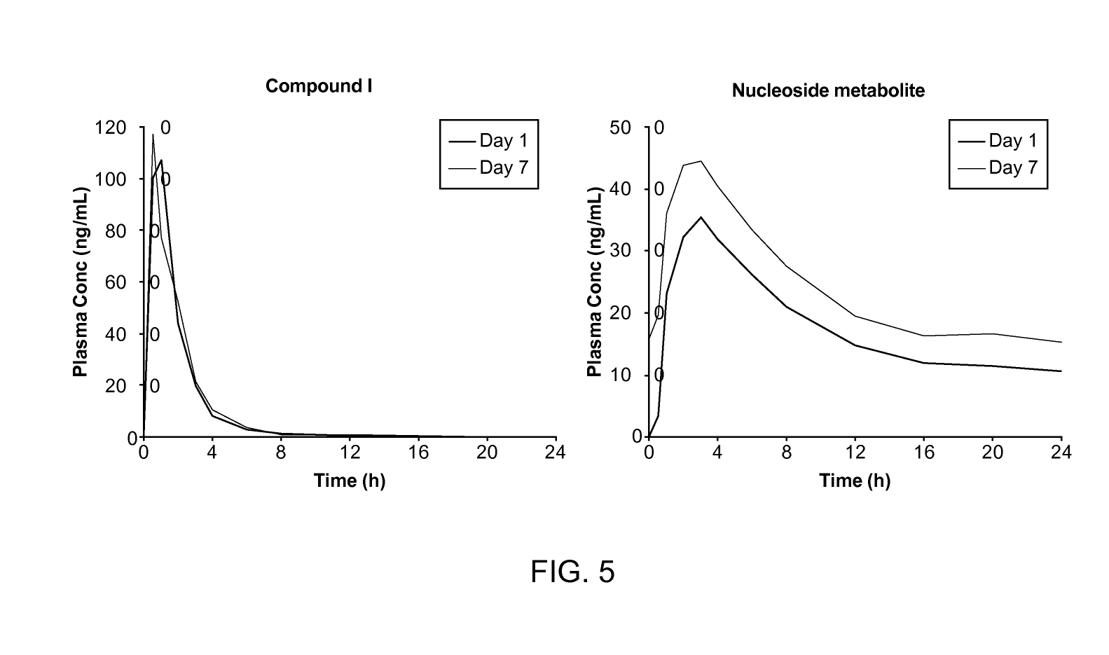

Summary of pharmacokinetic parameters of Compound I and the corresponding Nucleoside metabolite in healthy volunteers and HCV-infected subjects is shown in Tables C and D, and FIGS. 3-5.

TABLE C
Summary PK parameters of Compound I in healthy volunteers and HCV-infected subjects

| | Healthy volunteers | | | | HCV-infected subjects | | | |
|---|---|---|---|---|---|---|---|---|
| Dose | Cₘₐₓ | Tₘₐₓ | AUC_{∞} | t_{1/2} | Cₘₐₓ | Tₘₐₓ | AUC_{∞} | t_{1/2} |
| (mg) | (ng/mL) | (h) | ( ng/mL × h) | (hr) | (ng/mL) | (h) | (ng/mL × h) | (hr) |
| 10 | 17.4 ± 6.64 | 0.8 | 26.0 ± 7.60 | 0.87 ± 0.25 | 26.1 ± 11.9 | 0.5 | 40.0 ± 12.6 | 1.47 ± 0.70 |
| | | (0.5-1.0) | | | | (0.5-1.0) | | |
| 25 | 28.8 ± 13.4 | 1.0 | 51.2 ± 12.8 | 0.91 ± 0.26 | 68.4 ± 18.7 | 1.0 | 140 ± 68.8 | 0.97 ± 0.29 |
| | | (0.5-3.0) | | | | (0.5-1.0) | | |
| 50 | 82.4 ± 61.6 | 1.0 | 162 ± 77.2 | 1.04 ± 0.34 | 91.9 ± 10.7 | 1.0 | 197 ± 40.1 | 1.19 ± 0.46 |
| | | (0.5-1.0) | | | | (0.5-1.0) | | |
| 150 | 667 ± 324 | 1.0 | 1106 ± 690 | 1.16 ± 0.24 | 297 ± 34.0 | 0.5 | 664 ± 160 | 1.13 ± 0.29 |
| | | (0.5-2.0) | | | | (0.5-1.0) | | |
| 300 | 924 ± 441 | 0.5 | 1684 ± 725 | 1.19 ± 0.34 | 367 ± 537 | 1.0 | 818 ± 256 | 1.58 ± 0.02 |
| | | (0.5-2.0) | | | | (0.5-1.0) | | |
| 300 | 1292 ± 333 | 0.8 | 2167 ± 660 | 1.88 ± 0.72 | | | | |
| QD | | (0.5-1.0) | | | | | | |
| D1 | | | | | | | | |
| 300 | 1285 ± 238 | 0.5 | 2198 ± 494 | 2.76 ± 1.79 | | | | |
| QD | | (0.5-2.0) | | | | | | |
| D7 | | | | | | | | |

Parameters are presented as mean ± SD except for Tₘₐₓ where median (min-max) is shown.
¹ AUC_{24 h}

TABLE D
Summary PK parameters of the corresponding Nucleoside metabolite in healthy volunteers and HCV-infected subjects

| | Healthy volunteers | | | | | HCV-infected subjects | | | | |
|---|---|---|---|---|---|---|---|---|---|---|
| Dose | Cₘₐₓ | Tₘₐₓ | AUC_{∞} | t_{1/2} | C_{24 h} | Cmax | Tₘₐₓ | AUC_{∞} | t_{1/2} | C_{24 h} |
| (mg) | (ng/mL) | (h) | (ng/mL × h) | (hr) | (ng/mL) | (ng/mL) | (h) | (ng/mL × h) | (hr) | (ng/mL) |
| 10 | 17.8 ± 3.38 | 3.0 | 509 ± 188 | 22.6 ± 1.46 | 8.49 ± 3.35 | 15.3 ± 2.21 | 4.0 | 647 ± 148 | 20.1 ± 0.67 | 10.9 ± 2.24 |
| | | (2.0-6.0) | | | | | (4.0-6.0) | | | |
| 25 | 40.7 ± 5.38 | 3.5 | 1446 ± 384 | 24.3 ± 2.34 | 21.0 ± 6.68 | 47.0 ± 3.35 | 4.0 | 1626 ± 76.1 | 21.4 ± 1.28 | 21.0 ± 6.68 |
| | | (2.0-4.0) | | | | | (3.0-4.0) | | | |
| 50 | 71.9 ± 11.3 | 4.0 | 2475 ± 209 | 23.7 ± 3.39 | 37.5 ± 4.60 | 77.3 ± 4.01 | 2.0 | 2061 ± 224 | 22.7 ± 4.07 | 30.4 ± 4.36 |
| | | (2.0-6.0) | | | | | (2.0-4.0) | | | |
| 150 | 216 ± 51.6 | 2.5 | 5568 ± 759 | 29.3 ± 4.41 | 69.1 ± 13.3 | 184 ± 22.5 | 3.0 | 5160 ± 1173 | 26.6 ± 0.10 | 71.3 ± 22.5 |
| | | (2.0-6.0) | | | | | (2.0-4.0) | | | |
| 300 | 332 ± 55.6 | 3.5 | 8629 ± 2756 | 28.4 ± 2.11 | 101 ± 37.2 | 347 ± 103 | 4.0 | 9741 ± 3094 | 29.1 + 2.52 | 124 ± 43.0 |
| | | (3.0-4.0) | | | | | (1.0-6.0) | | | |
| 300 | 363 ± 276 | 3.0 | 4245 ± 707¹ | | 106 ± 34.2 | | | | | |
| QD | | (2.0-3.0) | | | | | | | | |
| D1 | | | | | | | | | | |
| 300 | 455 ± 65.6 | 2.5 | 5793 ± 1107¹ | 32.3 ± 4.55 | 154 ± 41.1 | | | | | |
| QD | | (1.0-4.0) | | | | | | | | |
| D7 | | | | | | | | | | |

Parameters are presented as mean ± SD except for Tₘₐₓ where median (min-max) is shown;
¹AUC_{24 h} or AUCₛₛ

While the claimed subject matter has been described in terms of various embodiments, the skilled artisan will appreciate that various modifications, substitutions, omissions, and changes may be made without departing from the spirit thereof. Accordingly, it is intended that the scope of the claimed subject matter is limited solely by the scope of the following claims, including equivalents thereof.

1. A Compound I in a form selected from the group consisting of: Form I, II, III, IV, V, VI, VII, and VIII, wherein Compound I has the following structure:

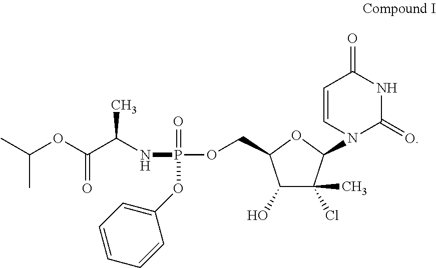 [SMILES: CC(C)OC(=O)[C@@H](C)N[P@@](=O)(OC[C@H]1O[C@@H](n2ccc(=O)[nH]c2=O)[C@](C)(Cl)[C@@H]1O)Oc1ccccc1]

2. Compound I of claim 1 where the compound is in substantially pure crystalline form.

3. Compound I of claim 1, wherein said compound is Form I, and said Form I is characterized by an X-ray powder diffraction pattern obtained using copper K_{α} radiation which comprises 2Θ values in degrees of about 6.8, about 15.7, and about 18.5.

4. Compound I of claim 2, wherein said Form I is characterized by an X-ray powder diffraction pattern obtained using copper K_{α} radiation which comprises 2Θ values in degrees of about 6.8, about 15.7, about 18.5, about, 18.0 about 13.6, and about 10.8.

5. Compound I of claim 3, wherein said Form I is characterized by an X-ray powder diffraction pattern obtained using copper K_{α} radiation which comprises 2Θ values in degrees of about 6.8, about 15.7, about 18.5, about 10.8, about 13.6, about 18.0, about 7.3, about 8.2, and about 19.3.

6. Compound I of claim 4, wherein said Form I is characterized by an X-ray powder diffraction pattern obtained using copper K_{α} radiation which comprises 2Θ values in degrees of about 6.8, about 15.7, about 18.5, about 10.8, about 13.6, about 18.0, about 7.3, about 8.2, about 19.3, about 16.8, about 19.7, about 21.4, about 21.8, about 23.6, about 24.4, about 24.8 and about 26.5.

7. Compound I of claim 1 wherein said compound is Form I, and said From I is characterized by a solid state ¹³C NMR spectrum having at least three peaks selected from the following: about 175.2 ppm, about 167.3 ppm, about 150.7 ppm, about 130.0 ppm, about 103.5 ppm, about 80.5 ppm, about 69.1 ppm, about 62.9 ppm, and about 49.2 ppm.

8. Form I of claim 1, which is substantially free of an amorphous form.

9. A composition comprising Compound I: 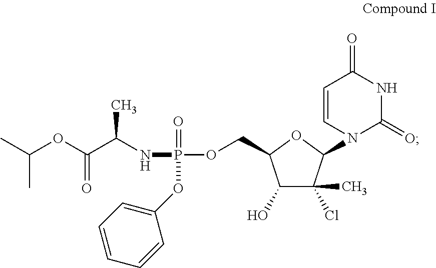 [SMILES: CC(C)OC(=O)[C@@H](C)N[P@@](=O)(OC[C@H]1O[C@@H](n2ccc(=O)[nH]c2=O)[C@](C)(Cl)[C@@H]1O)Oc1ccccc1]
or a pharmaceutically acceptable salt thereof, in an amount of about 25 to about 500 mg, and a pharmaceutically acceptable carrier.

10. The composition of claim 9, wherein said Compound I or a pharmaceutically acceptable salt thereof is provided in amount of about 300 mg.

11. The composition of claim 10, wherein said Compound I or a pharmaceutically acceptable salt thereof is provided in amount of about 450 mg.

12. The composition of claim 10 provided as a capsule.

13. The composition of claim 11 provided as a capsule.

14. The composition of claim 10 provided as a tablet.

15. The composition of claim 11 provided as a tablet.

16. The composition of claim 9, wherein Compound I is in a form selected from the group consisting of: Form I, II, III, IV, V, VI, VII, and VIII.

17. A method for the treatment of a patient infected with hepatitis C virus, comprising administering to said patient an effective amount of Compound I of claim 1.

18. The method of claim 17, where said patient is a human.
